(19) United States
(12) Patent Application Publication
(10) Pub. No.: US 2009/0062269 A1
(43) Pub. Date: Mar. 5, 2009

(54) Niacin Receptor Agonists, Compositions Containing Such Compounds and Methods of Treatment

(76) Inventors: Subharekha Raghavan, Teaneck, NJ (US); Darby Rye Schmidt, Clark, NJ (US); Steven L. Colletti, Princeton Junction, NJ (US); Abigail Lee Smenton, Brooklyn, NY (US)

Correspondence Address: MERCK AND CO., INC, P O BOX 2000, RAHWAY, NJ, 07065-0907, US

(21) Appl. No.: 12/223,492

(22) PCT Filed: Feb. 2, 2007
(86) PCT No.: PCT/US2007/002994

§ 371 Date: Jul. 31, 2008

Related U.S. Application Data
(60) Provisional application No. 60/765,853, filed on Feb. 7, 2006.

(51) Int. Cl. A61K 31/195; C07D 277/30; C07C 229/46; C07D 211/78; C07D 213/61; C07D 213/56; A61K 31/444; A61K 31/4418; A61K 31/381; A61K 31/538; A61K 31/4375; A61K 31/404; A61K 31/437; A61P 3/10; A61K 31/426; A61K 31/4439; C07D 413/14; C07D 403/04; C07D 401/04; C07D 333/24; A61P 3/00

(52) U.S. Cl. 514/230.5; 548/204; 562/455; 546/309; 546/335; 546/256; 546/269.4; 546/275.4; 549/77; 514/563; 514/333; 514/341; 514/340; 514/357; 514/365; 514/438; 514/292; 514/411; 514/294

(57) ABSTRACT

The present invention encompasses compounds of Formula I: as well as pharmaceutically acceptable salts and hydrates thereof, that are useful for treating atherosclerosis, dyslipidemias and the like. Pharmaceutical compositions and methods of use are also included.

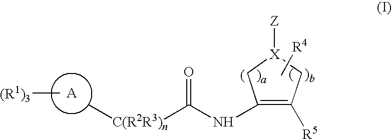

### PRIORITY CLAIM

This application is a §371 National Stage Application of PCT/US2007/002994, filed on Feb. 2, 2007, which claims priority from U.S. Provisional Application Ser. No. 60/765,853, filed on Feb. 7, 2006.

### BACKGROUND OF THE INVENTION

The present invention relates to aryl-cycloalkene compounds, compositions containing such compounds and methods of treatment or prevention using such compounds, primarily in disease and conditions relating to dyslipidemias. Dyslipidemia is a condition wherein serum lipids are abnormal. Elevated cholesterol and low levels of high density lipoprotein (HDL) are independent risk factors for atherosclerosis associated with a greater-than-normal risk of atherosclerosis and cardiovascular disease. Factors known to affect serum cholesterol include genetic predisposition, diet, body weight, degree of physical activity, age and gender. While cholesterol in normal amounts is a vital building block for cell membranes and essential organic molecules, such as steroids and bile acids, cholesterol in excess is known to contribute to cardiovascular disease. For example, cholesterol, through its relationship with foam cells, is a primary component of plaque which collects in coronary arteries, resulting in the cardiovascular disease termed atherosclerosis.

Traditional therapies for reducing cholesterol include medications such as statins (which reduce production of cholesterol by the body). More recently, the value of nutrition and nutritional supplements in reducing blood cholesterol has received significant attention. For example, dietary compounds such as soluble fiber, vitamin E, soy, garlic, omega-3 fatty acids, and niacin have all received significant attention and research funding.

Niacin or nicotinic acid (pyridine-3-carboxylic acid) is a drug that reduces coronary events in clinical trials. It is commonly known for its effect in elevating serum levels of high density lipoproteins (HDL). Importantly, niacin also has a beneficial effect on other lipid profiles. Specifically, it reduces low density lipoproteins (LDL), very low density lipoproteins (VLDL), and triglycerides (TG). However, the clinical use of nicotinic acid is limited by a number of adverse side-effects including cutaneous vasodilation, sometimes called flushing.

Despite the attention focused on traditional and alternative means for controlling serum cholesterol, serum triglycerides, and the like, a significant portion of the population has total cholesterol levels greater than about 200 mg/dL, and are thus candidates for dyslipidemia therapy. There thus remains a need in the art for compounds, compositions and alternative methods of reducing total cholesterol, serum triglycerides, and the like, and raising HDL.

The present invention relates to compounds that have been discovered to have effects in modifying serum lipid levels.

The invention thus provides compositions for effecting reduction in total cholesterol and triglyceride concentrations and raising HDL, in accordance with the methods described.

Consequently one object of the present invention is to provide a nicotinic acid receptor agonist that can be used to treat dyslipidemias, atherosclerosis, diabetes, metabolic syndrome and related conditions while minimizing the adverse effects that are associated with niacin treatment.

Yet another object is to provide a pharmaceutical composition for oral use.

These and other objects will be apparent from the description provided herein.

### SUMMARY OF THE INVENTION

A compound represented by formula I:

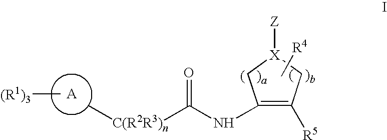

or a pharmaceutically acceptable salt or solvate thereof is disclosed wherein:

X represents a carbon or nitrogen atom;

Z represents Aryl and Heteroaryl, said Aryl and Heteroaryl being optionally substituted with 1-3 groups, 1-3 of which are halo, and 0-1 of which are selected from the group consisting of: OH, NH₂, C₁₋₃alkyl, C₁₋₃alkoxy, haloC₁₋₃alkyl and haloC₁₋₃alkoxy groups;

R⁴ is H, fluoro, or C₁₋₃alkyl optionally substituted with 1-3 groups, 0-3 of which are halo, and 0-1 of which are selected from the group consisting of: OC₁₋₃alkyl, OH, NH₂, NHC₁₋₃alkyl, N(C₁₋₃alkyl)₂, CN and Hetcy;

a and b are each integers 1 or 2, such that the sum of a and b is 3;

ring A represents a 6-10 membered Aryl, or a 5-13 membered Heteroaryl group, said Heteroaryl group containing at least one heteroatom selected from O, S, S(O), S(O)₂ and N, and optionally containing 1 other heteroatom selected from O and S, and optionally containing 1-3 additional N atoms, with up to 5 heteroatoms being present;

each R² and R³ is independently H, C₁₋₃alkyl, haloC₁₋₃alkyl, OC₁₋₃alkyl, haloC₁₋₃alkoxy, OH or F;

n represents an integer of from 2 to 4;

R⁵ represents —CO₂H,

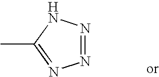

—C(O)NHSO₂R^{e} wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;

and each R⁺ is H or is independently selected from the group consisting of:

a) halo, OH, CO₂H, CN, NH₂, S(O)₀₋₂R^{e}, C(O)R^{e}, OC(O)R^{e} and CO₂R^{e}, wherein R^{e} is as previously defined;

b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;

c) NHC₁₋₄alkyl and N(C₁₋₄alkyl)₂, the alkyl portions of which are optionally substituted as set forth in (b) above;

d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;

e) NR′C(O)R″, NR′SO₂R″, NR′CO₂R″ and NR′C(O)NR″R′″ wherein:

R′ represents H, C₁₋₃alkyl or haloC₁₋₃alkyl,

R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₁₋₄alkoxy groups;
   (b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;

and R′″ representing H or R″;

f) phenyl or a 5-6 membered Heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl and the like. Cycloalkyl is a subset of alkyl; if no number of atoms is specified, 3-7 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Aryl” (Ar) means mono- and bicyclic aromatic rings containing 6-10 carbon atoms. Examples of aryl include phenyl, naphthyl, indenyl and the like.

“Heteroaryl” (HAR) unless otherwise specified, means mono-, bicyclic and tricyclic aromatic ring systems containing at least one heteroatom selected from O, S, S(O), SO₂ and N, with each ring containing 5 to 6 atoms. HAR groups may contain from 5-14, preferably 5-13 atoms. Examples include, but are not limited to, pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, benzopyrazolyl, benzotriazolyl, furo(2,3-b)pyridyl, benzoxazinyl, tetrahydrohydroquinolinyl, tetrahydroisoquinolinyl, quinolyl, isoquinolyl, indolyl, dihydroindolyl, quinoxalinyl, quinazolinyl, naphthyridinyl, pteridinyl, 2,3-dihydrofuro(2,3-b)pyridyl and the like. Heteroaryl also includes aromatic carbocyclic or heterocyclic groups fused to heterocycles that are non-aromatic or partially aromatic, and optionally containing a carbonyl. Examples of additional heteroaryl groups include indolinyl, dihydrobenzofuranyl, dihydrobenzothiophenyl, dihydrobenzoxazolyl, and aromatic heterocyclic groups fused to cycloalkyl rings. Examples also include the following:

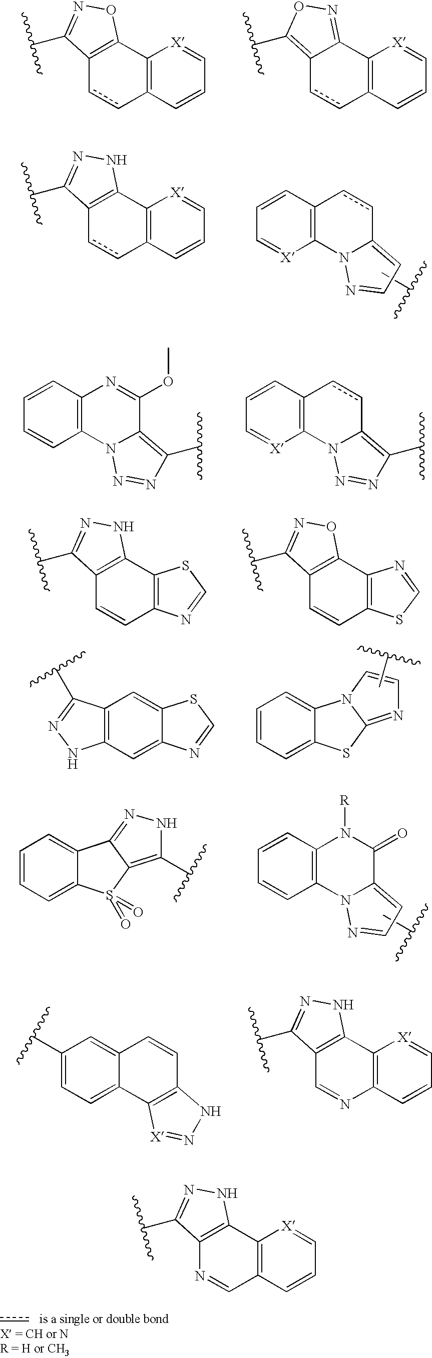

Heteroaryl also includes such groups in charged form, e.g., pyridinium.

“Heterocyclyl” (Hetcy) unless otherwise specified, means mono- and bicyclic saturated rings and ring systems containing at least one heteroatom selected from N, S and O, each of said ring having from 3 to 10 atoms in which the point of attachment may be carbon or nitrogen. Examples of “heterocyclyl” include, but are not limited to, azetidinyl, pyrrolidinyl, piperidinyl, piperazinyl, imidazolidinyl, tetrahydrofuranyl, 1,4-dioxanyl, morpholinyl, thiomorpholinyl, tetrahydrothienyl and the like. Heterocycles can also exist in tautomeric forms, e.g., 2- and 4-pyridones. Heterocycles moreover includes such moieties in charged form, e.g., piperidinium.

“Halogen” (Halo) includes fluorine, chlorine, bromine and iodine.

The phrase “in the absence of substantial flushing” refers to the side effect that is often seen when nicotinic acid is administered in therapeutic amounts. The flushing effect of nicotinic acid usually becomes less frequent and less severe as the patient develops tolerance to the drug at therapeutic doses, but the flushing effect still occurs to some extent and can be transient. Thus, “in the absence of substantial flushing” refers to the reduced severity of flushing when it occurs, or fewer flushing events than would otherwise occur. Preferably, the incidence of flushing (relative to niacin) is reduced by at least about a third, more preferably the incidence is reduced by half, and most preferably, the flushing incidence is reduced by about two thirds or more. Likewise, the severity (relative to niacin) is preferably reduced by at least about a third, more preferably by at least half, and most preferably by at least about two thirds. Clearly a one hundred percent reduction in flushing incidence and severity is most preferable, but is not required.

One aspect of the invention relates to a compound represented by formula I:

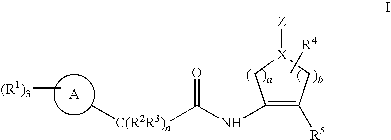

or a pharmaceutically acceptable salt or solvate thereof wherein:

X represents a carbon or nitrogen atom;

Z represents Aryl and Heteroaryl, said Aryl and Heteroaryl being optionally substituted with 1-3 groups, 1-3 of which are halo, and 0-1 of which are selected from the group consisting of: OH, NH₂, C₁₋₃alkyl, C₁₋₃alkoxy, haloC₁₋₃alkyl and haloC₁₋₃alkoxy groups;

R⁴ is H, fluoro, or C₁₋₃alkyl optionally substituted with 1-3 groups, 0-3 of which are halo, and 0-1 of which are selected from the group consisting of: OC₁₋₃alkyl, OH, NH₂, NHC₁₋₃alkyl, N(C₁₋₃alkyl)₂, CN and Hetcy;

a and b are each integers 1 or 2, such that the sum of a and b is 3;

ring A represents a 6-10 membered Aryl, or a 5-13 membered Heteroaryl group, said Heteroaryl group containing at least one heteroatom selected from O, S, S(O), S(O)₂ and N, and optionally containing 1 other heteroatom selected from O and S, and optionally containing 1-3 additional N atoms, with up to 5 heteroatoms being present;

each R² and R³ is independently H, C₁₋₃alkyl, haloC₁₋₃alkyl, OC₁₋₃alkyl, haloC₁₋₃alkoxy, OH or F;

n represents an integer of from 2 to 4;

R⁵ represents —CO₂H,

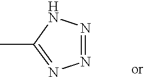

—C(O)NHSO₂R^{e} wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;

and each R¹ is H or is independently selected from the group consisting of:

a) halo, OH, CO₂H, CN, NH₂, S(O)₀₋₂R^{e}, C(O)R^{e}, OC(O)R^{e} and CO₂R^{e}, wherein R^{e} is as previously defined;

b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;

c) NHC₁₋₄alkyl and N(C₁₋₄alkyl)₂, the alkyl portions of which are optionally substituted as set forth in (b) above;

d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;

e) NR′C(O)R″, NR′SO₂R″, NR′CO₂R″ and NR′C(O)NR″R′″ wherein:

R′ represents H, C₁₋₃alkyl or haloC₁₋₃alkyl,

R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₁₋₄alkoxy groups;
   (b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;

and R′″ representing H or R″;

f) phenyl or a 5-6 membered Heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above.

A subset of compounds that is of interest relates to compounds of formula I wherein ring

A represents an Aryl group, a 5-6 membered monocyclic Heteroaryl group or a 9-13 membered bicyclic or tricyclic Heteroaryl group. Within this subset of compounds, all other variables are as defined with respect to formula I.

In particular, a subset of compounds that is of interest relates to compounds of formula I wherein ring A is selected from the group consisting of:

Aryl selected from phenyl and naphthyl;

HAR selected from the group consisting of: pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, benzopyrazolyl, benzotriazolyl, furo(2,3-b)pyridyl, benzoxazinyl, tetrahydrohydroquinolinyl, tetrahydroisoquinolinyl, quinolyl, isoquinolyl, indolyl, dihydroindolyl, quinoxalinyl, quinazolinyl, naphthyridinyl, pteridinyl, 2,3-dihydrofuro(2,3-b)pyridyl indolinyl, dihydrobenzofuranyl, dihydrobenzothiophenyl, dihydrobenzoxazolyl, or a member selected from the group consisting of:

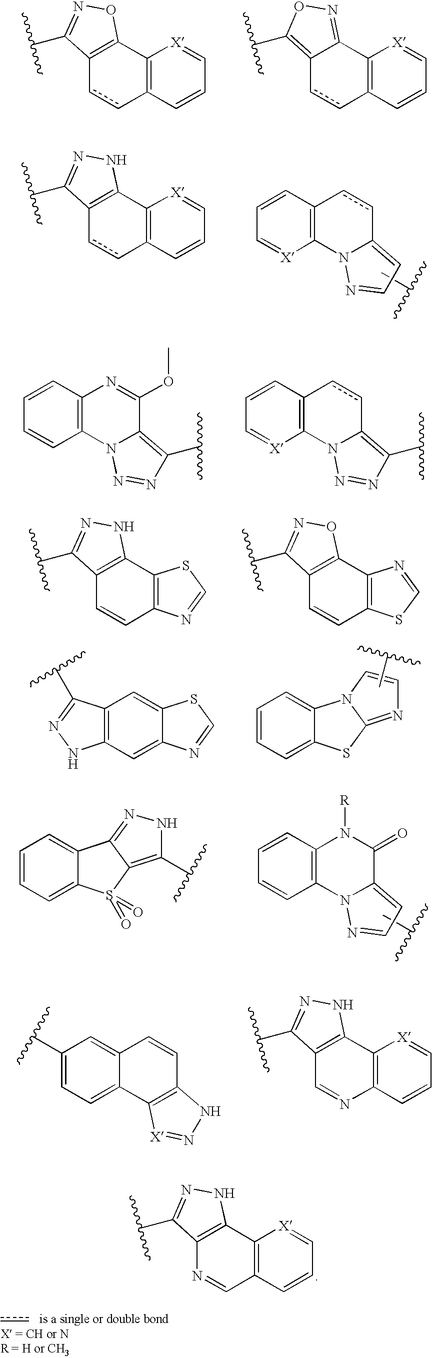

More particularly, a subset of compounds that is of interest relates to compounds of formula I wherein ring A is selected from the group consisting of: phenyl, naphthyl, pyrrolyl, isoxazolyl, isothiazolyl, pyrazolyl, pyridyl, oxazolyl, oxadiazolyl, thiadiazolyl, thiazolyl, imidazolyl, triazolyl, furanyl, and thienyl. Within this subset of compounds, all other variables are as defined with respect to formula I.

Even more particularly, a subset of compounds that is of interest relates to compounds of formula I wherein ring A is selected from the group consisting of: phenyl, naphthyl, oxadiazolyl, pyrazolyl and thiazolyl. Within this subset of compounds, all other variables are as defined with respect to formula I.

Another subset of compounds that is of interest relates to compounds of formula I wherein each R¹ is H or is independently selected from the group consisting of:

a) halo, OH, CO₂H, CN, NH₂, S(O)₀₋₂R^{e}, C(O)R^{e}, OC(O)R^{e} and CO₂R^{e}, wherein R^{e} is as previously defined;

b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;

c) phenyl or a 5-6 membered Heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above. Within this subset of compounds, all other variables are as defined with respect to formula I.

More particularly, an aspect of the invention that is of interest relates to compounds of formula I wherein each R¹ is H or is independently selected from the group consisting of:

a) halo, OH, CO₂H, CN, NH₂, S(O)₀₋₂R^{e}, C(O)R^{e}, OC(O)R^{e} and CO₂R^{e}, wherein R^{e} is as previously defined; and

b) phenyl or a 5-6 membered Heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above. Within this subset of compounds, all other variables are as defined with respect to formula I.

Even more particularly, an aspect of the invention that is of interest relates to compounds of formula I wherein each R¹ is H or is independently selected from the group consisting of:

a) halo, OH, CN, NH₂, and

b) phenyl or a 5-6 membered Heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CN; NH₂ and;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN; and
iii) NR′C(O)R″, NR′SO₂R″, NR′CO₂R″ and NR′C(O)NR″R′″ wherein R′, R″ and R′″ are as described above. Within this subset of compounds, all other variables are as defined with respect to formula I.

Another subset of compounds that is of interest relates to compounds of formula I wherein X represents a carbon atom. Within this subset of compounds, all other variables are as defined with respect to formula I.

Another subset of compounds that is of interest relates to compounds of formula I wherein X represents a nitrogen atom. Within this subset of compounds, all other variables are as defined with respect to formula I.

Another subset of compounds that is of interest relates to compounds of formula I wherein R² and R³ are independently H, C₁₋₃alkyl or haloC₁₋₃alkyl. Within this subset of compounds, all other variables are as defined with respect to formula I.

More particularly, a subset of compounds that is of interest relates to compounds of formula I wherein R² and R³ are independently H or methyl. Within this subset of compounds, all other variables are as defined with respect to formula I.

Another subset of compounds that is of interest relates to compounds of formula I wherein n is 2. Within this subset of compounds, all other variables are as defined with respect to formula I.

Another subset of compounds that is of interest relates to compounds of formula I wherein Z is Aryl optionally substituted with 1-3 halo groups and 0-1 groups selected from C₁₋₃alkyl and haloC₁₋₃alkyl. Within this subset of compounds, all other variables are as defined with respect to formula I.

Another subset of compounds that is of interest relates to compounds of formula I wherein Z is Heteroaryl optionally substituted with 1-3 halo groups and 0-1 groups selected from C₁₋₃alkyl and haloC₁₋₃alkyl. Within this subset of compounds, all other variables are as defined with respect to formula I.

Another subset of compounds that is of interest relates to compounds of formula I wherein R⁴ is H, fluoro or methyl optionally substituted with 1-3 halo groups. Within this subset of compounds, all other variables are as defined with respect to formula I.

Another subset of compounds that is of interest relates to compounds of formula I wherein R⁵ represents —CO₂H. Within this subset of compounds, all other variables are as defined with respect to formula I.

Representative examples of species that are of interest are shown below in Table I. Within this subset of compounds, all other variables are as originally defined with respect to formula I.

TABLE 1

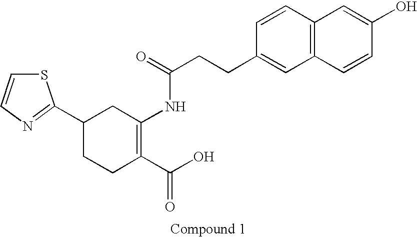

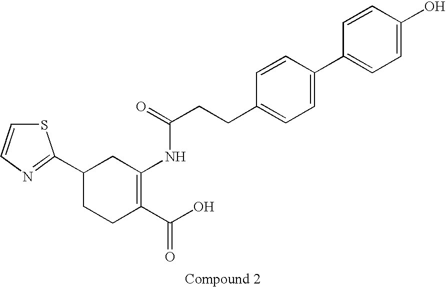

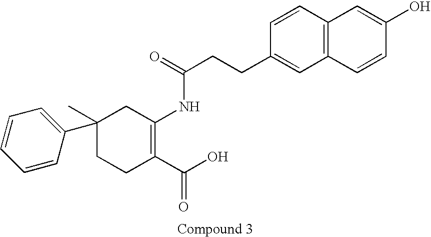

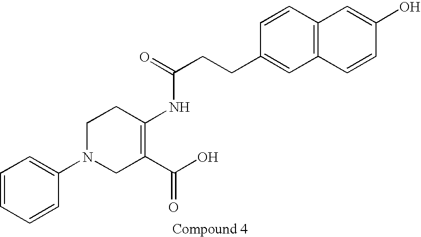

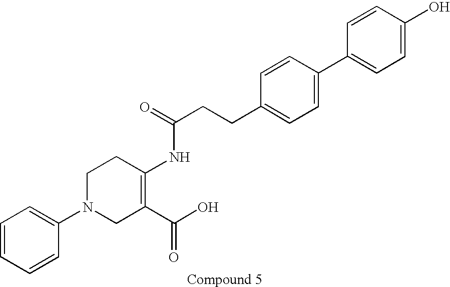

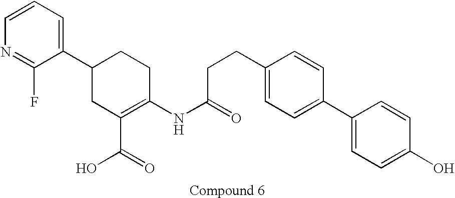

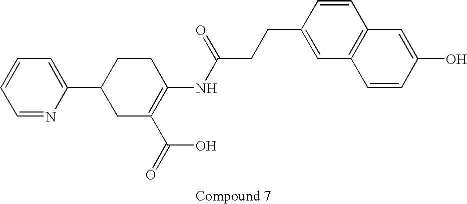

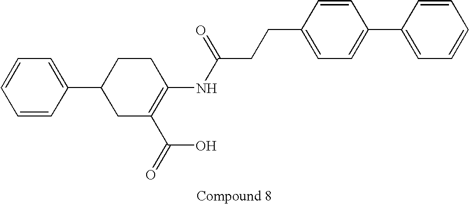

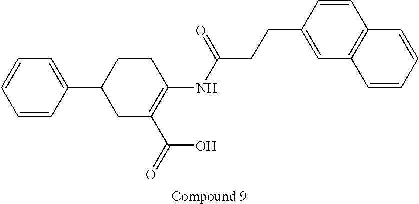

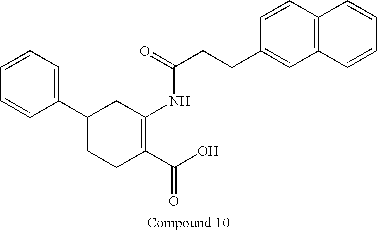

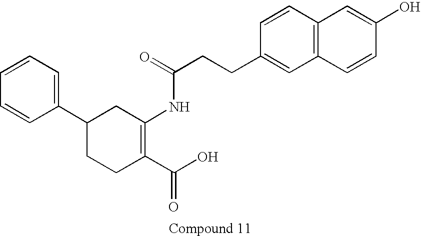

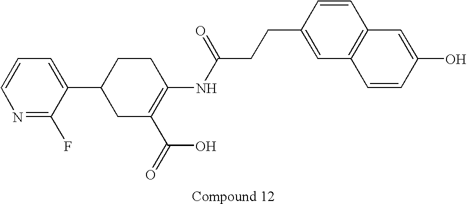

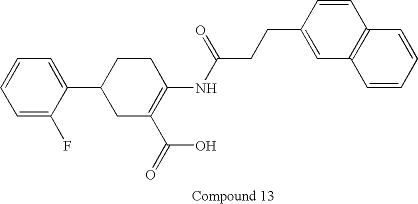

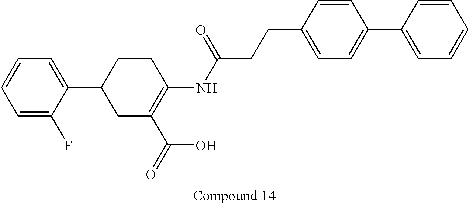

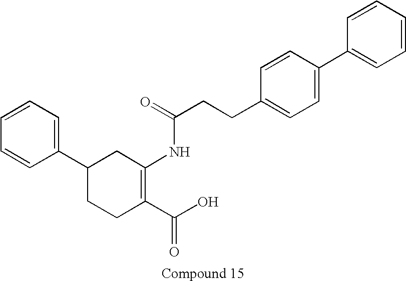

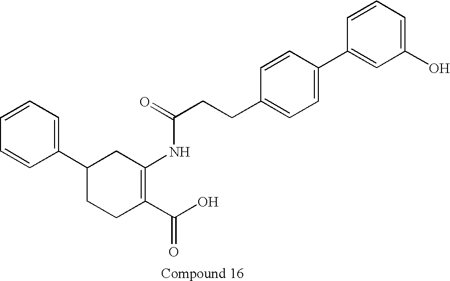

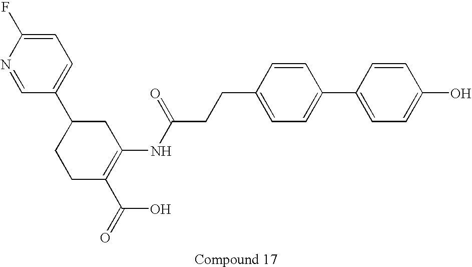

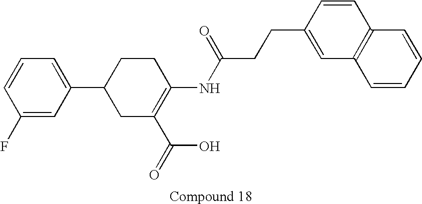

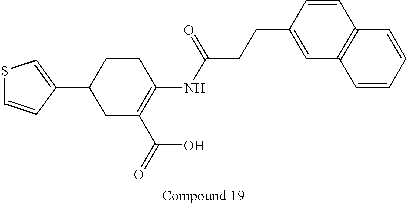

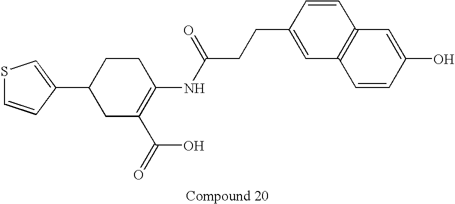

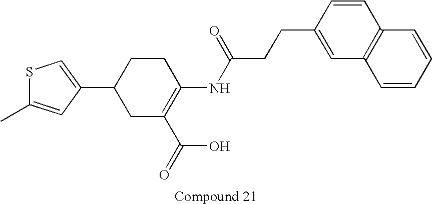

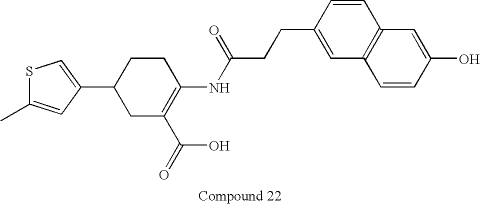

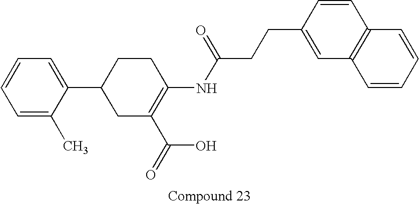

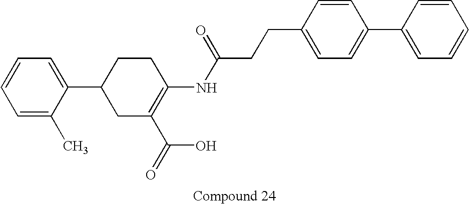

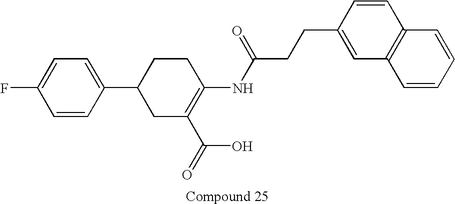

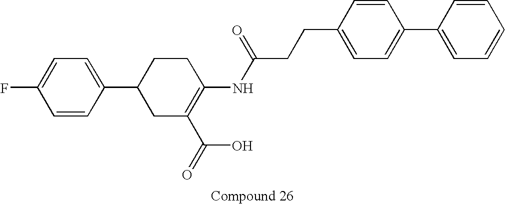

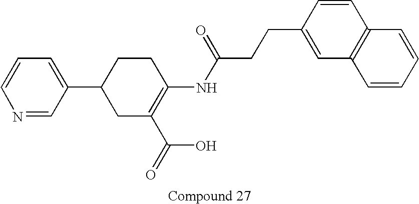

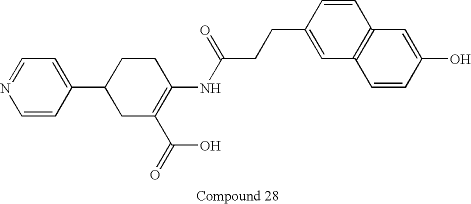

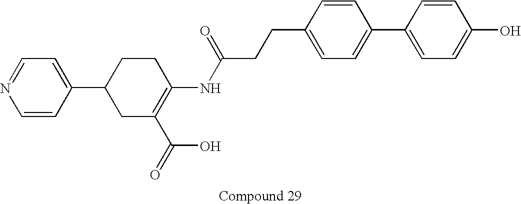

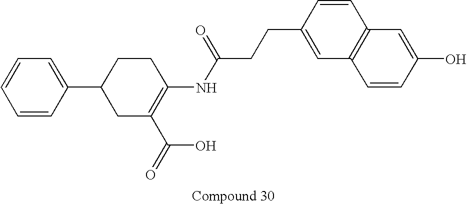

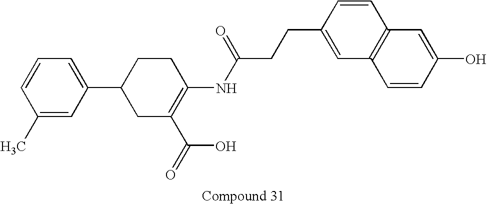

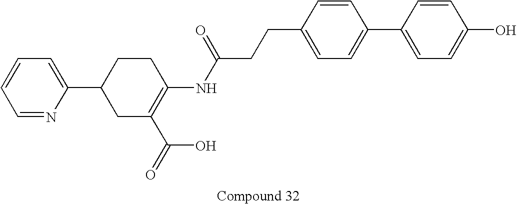

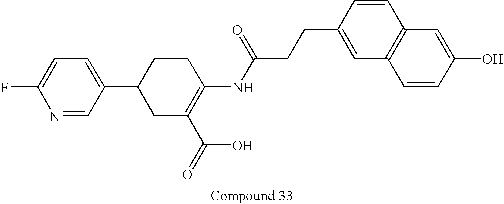

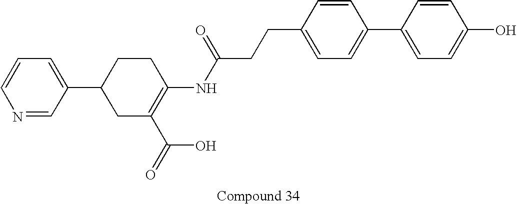

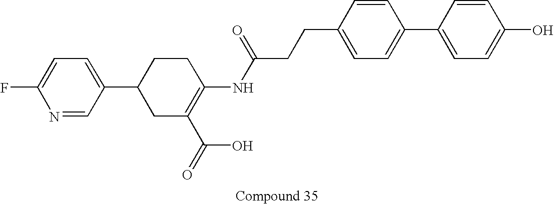

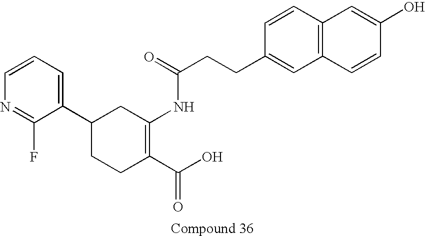

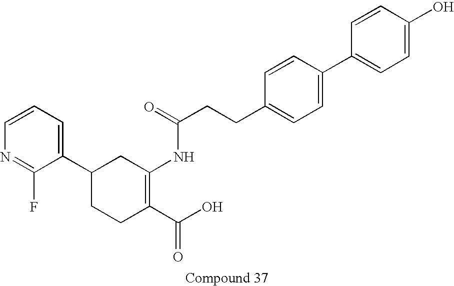

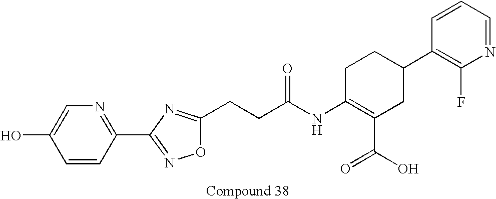

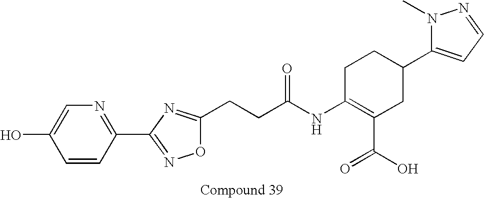

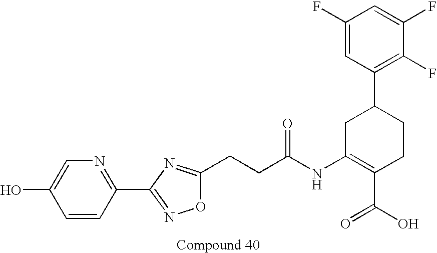

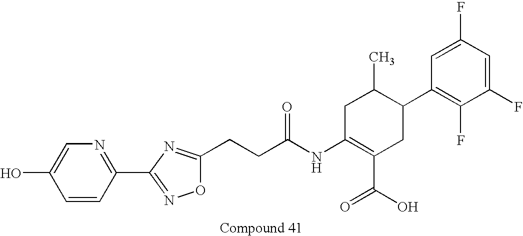

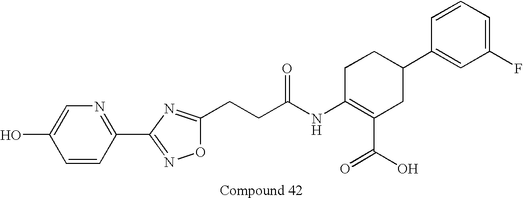

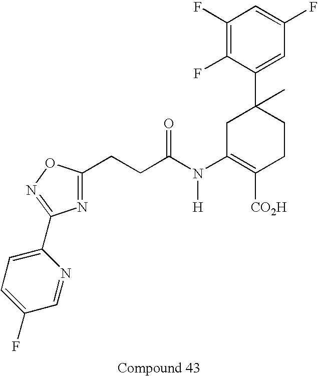

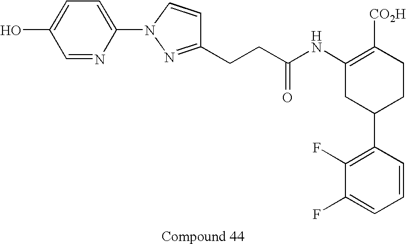

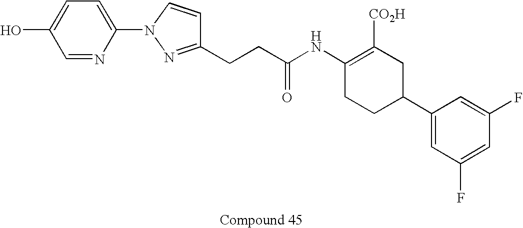

Pharmaceutically acceptable salts and solvates thereof are included as well.

All of the compounds of formula I contain asymmetric stereocenters and can thus occur as racemates and racemic mixtures, single enantiomers, diastereomeric mixtures and individual diastereomers. All such isomeric forms are included.

Moreover, chiral compounds possessing one stereocenter of general formula I, may be resolved into their enantiomers in the presence of a chiral environment using methods known to those skilled in the art. Chiral compounds possessing more than one stereocenter may be separated into their diastereomers in an achiral environment on the basis of their physical properties using methods known to those skilled in the art. Single diastereomers that are obtained in racemic form may be resolved into their enantiomers as described above.

If desired, racemic mixtures of compounds may be separated so that individual enantiomers are isolated. The separation can be carried out by methods well known in the art, such as the coupling of a racemic mixture of compounds of Formula I to an enantiomerically pure compound to form a diastereomeric mixture, which is then separated into individual diastereomers by standard methods, such as fractional crystallization or chromatography. The coupling reaction is often the formation of salts using an enantiomerically pure acid or base. The diasteromeric derivatives may then be converted to substantially pure enantiomers by cleaving the added chiral residue from the diastereomeric compound.

The racemic mixture of the compounds of Formula I can also be separated directly by chromatographic methods utilizing chiral stationary phases, which methods are well known in the art.

Alternatively, enantiomers of compounds of the general Formula I may be obtained by stereoselective synthesis using optically pure starting materials or reagents.

Some of the compounds described herein exist as tautomers, which have different points of attachment for hydrogen accompanied by one or more double bond shifts. For example, a ketone and its enol form are keto-enol tautomers. Or for example, a 2-hydroxyquinoline can reside in the tautomeric 2-quinolone form. The individual tautomers as well as mixtures thereof are included.

### Dosing Information

The dosages of compounds of formula I or a pharmaceutically acceptable salt or solvate thereof vary within wide limits. The specific dosage-regimen and levels for any particular patient will depend upon a variety of factors including the age, body weight, general health, sex, diet, time of administration, route of administration, rate of excretion, drug combination and the severity of the patient's condition. Consideration of these factors is well within the purview of the ordinarily skilled clinician for the purpose of determining the therapeutically effective or prophylactically effective dosage amount needed to prevent, counter, or arrest the progress of the condition. Generally, the compounds will be administered in amounts ranging from as low as about 0.01 mg/day to as high as about 2000 mg/day, in single or divided doses. A representative dosage is about 0.1 mg/day to about 1 g/day. Lower dosages can be used initially, and dosages increased to further minimize any untoward effects. It is expected that the compounds described herein will be administered on a daily basis for a length of time appropriate to treat or prevent the medical condition relevant to the patient, including a course of therapy lasting months, years or the life of the patient.

### Combination Therapy

One or more additional active agents may be administered with the compounds described herein. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as CRESTOR®; see U.S. Pat. No. 5,260,440); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; endothelial lipase inhibitors; bile acid sequestrants; LDL receptor inducers; platelet aggregation inhibitors, for example glycoprotein IIb/IIIa fibrinogen receptor antagonists and aspirin; human peroxisome proliferator activated receptor gamma (PPAR-gamma) agonists including the compounds commonly referred to as glitazones for example pioglitazone and rosiglitazone and, including those compounds included within the structural class known as thiazolidine diones as well as those PPAR-gamma agonists outside the thiazolidine dione structural class; PPAR-alpha agonists such as clofibrate, fenofibrate including micronized fenofibrate, and gemfibrozil; PPAR dual alpha/gamma agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; renin inhibitors, calcium channel blockers such as nifedipine and diltiazem; endothelin antagonists; agents that enhance ABCA1 gene expression; cholesteryl ester transfer protein (CETP) inhibiting compounds, 5-lipoxygenase activating protein (FLAP) inhibiting compounds, 5-lipoxygenase (5-LO) inhibiting compounds, farnesoid X receptor (FXR) ligands including both antagonists and agonists; Liver X Receptor (LXR)-alpha ligands, LXR-beta ligands, bisphosphonate compounds such as alendronate sodium; cyclooxygenase-2 inhibitors such as rofecoxib and celecoxib; and compounds that attenuate vascular inflammation.

Cholesterol absorption inhibitors can also be used in the present invention. Such compounds block the movement of cholesterol from the intestinal lumen into enterocytes of the small intestinal wall, thus reducing serum cholesterol levels. Examples of cholesterol absorption inhibitors are described in U.S. Pat. Nos. 5,846,966, 5,631,365, 5,767,115, 6,133,001, 5,886,171, 5,856,473, 5,756,470, 5,739,321, 5,919,672, and in PCT application Nos. WO 00/63703, WO 00/60107, WO 00/38725, WO 00/34240, WO 00/20623, WO 97/45406, WO 97/16424, WO 97/16455, and WO 95/08532. The most notable cholesterol absorption inhibitor is ezetimibe, also known as 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966.

Therapeutically effective amounts of cholesterol absorption inhibitors include dosages of from about 0.01 mg/kg to about 30 mg/kg of body weight per day, preferably about 0.1 mg/kg to about 15 mg/kg.

For diabetic patients, the compounds used in the present invention can be administered with conventional diabetic medications. For example, a diabetic patient receiving treatment as described herein may also be taking insulin or an oral antidiabetic medication. One example of an oral antidiabetic medication useful herein is metformin.

In the event that these niacin receptor agonists induce some degree of vasodilation, it is understood that the compounds of formula I may be co-dosed with a vasodilation suppressing agent. Consequently, one aspect of the methods described herein relates to the use of a compound of formula I or a pharmaceutically acceptable salt or solvate thereof in combination with a compound that reduces flushing. Conventional compounds such as aspirin, ibuprofen, naproxen, indomethacin, other NSAIDs, COX-2 selective inhibitors and the like are useful in this regard, at conventional doses. Alternatively, DP antagonists are useful as well. Doses of the DP receptor antagonist and selectivity are such that the DP antagonist selectively modulates the DP receptor without substantially modulating the CRTH2 receptor. In particular, the DP receptor antagonist ideally has an affinity at the DP receptor (i.e., Kᵢ) that is at least about 10 times higher (a numerically lower K; value) than the affinity at the CRTH2 receptor. Any compound that selectively interacts with DP according to these guidelines is deemed “Dselective”. This is in accordance with US Published Application No. 2004/0229844A1 published on Nov. 18, 2004, incorporated herein by reference.

Dosages for DP antagonists as described herein, that are useful for reducing or preventing the flushing effect in mammalian patients, particularly humans, include dosages ranging from as low as about 0.01 mg/day to as high as about 100 mg/day, administered in single or divided daily doses. Preferably the dosages are from about 0.1 mg/day to as high as about 1.0 g/day, in single or divided daily doses.

Examples of compounds that are particularly useful for selectively antagonizing DP receptors and suppressing the flushing effect include the following:

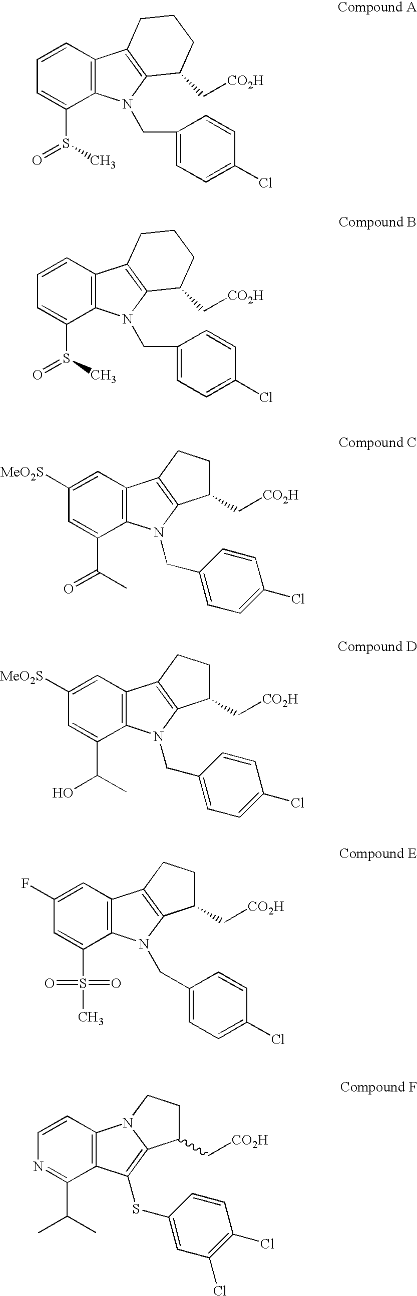

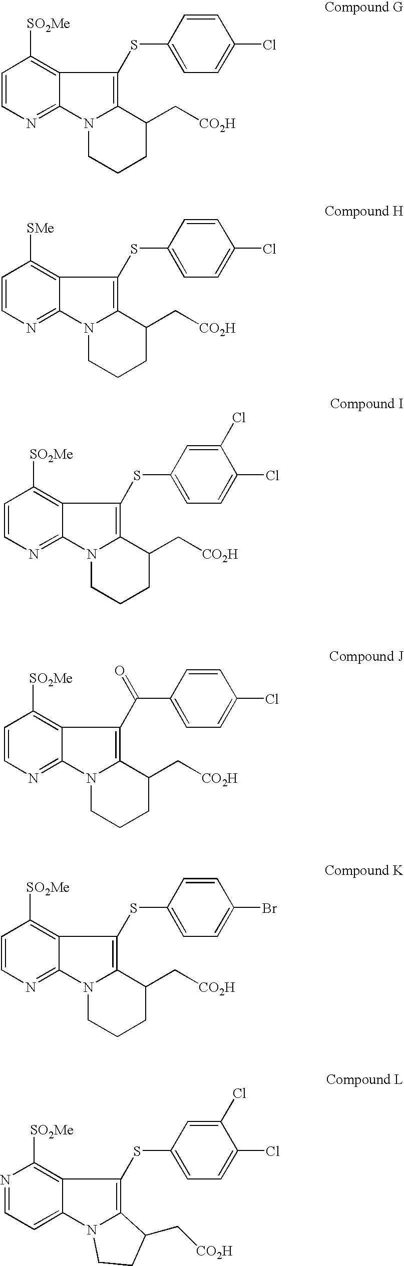

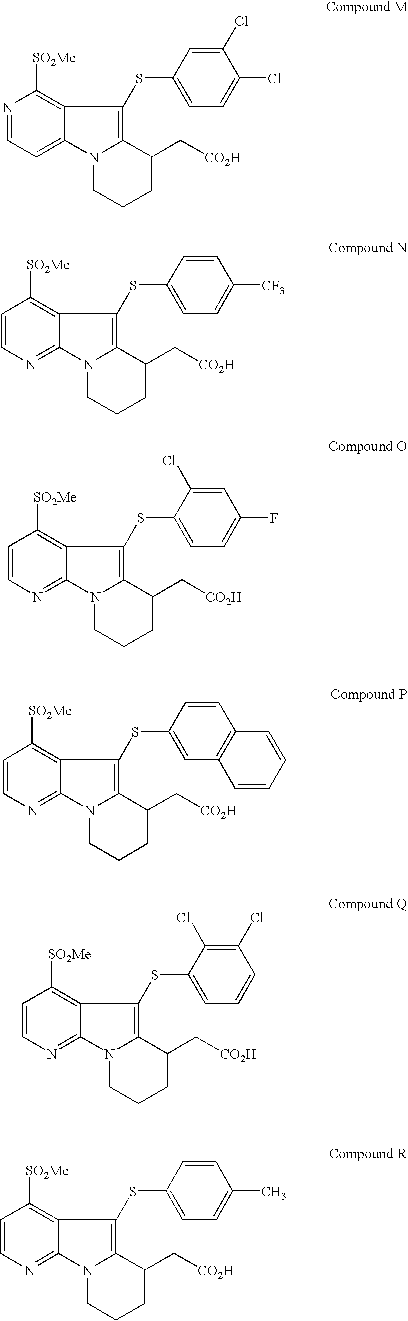

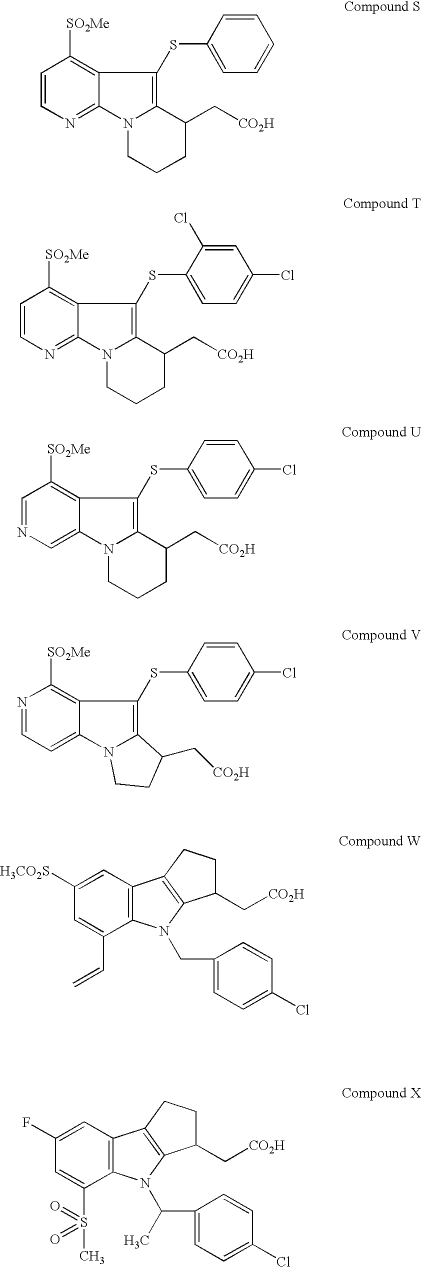

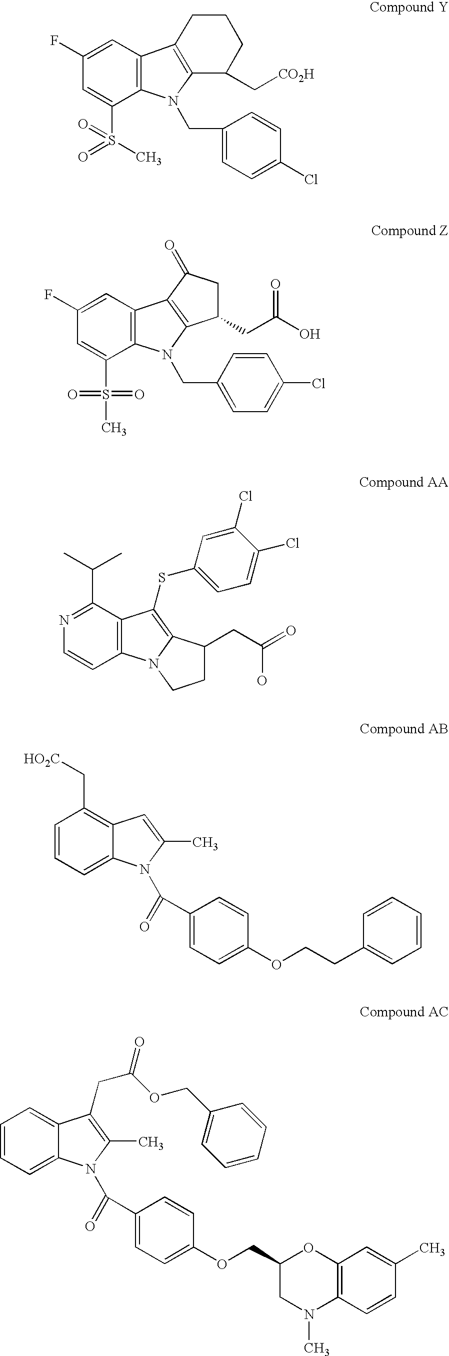

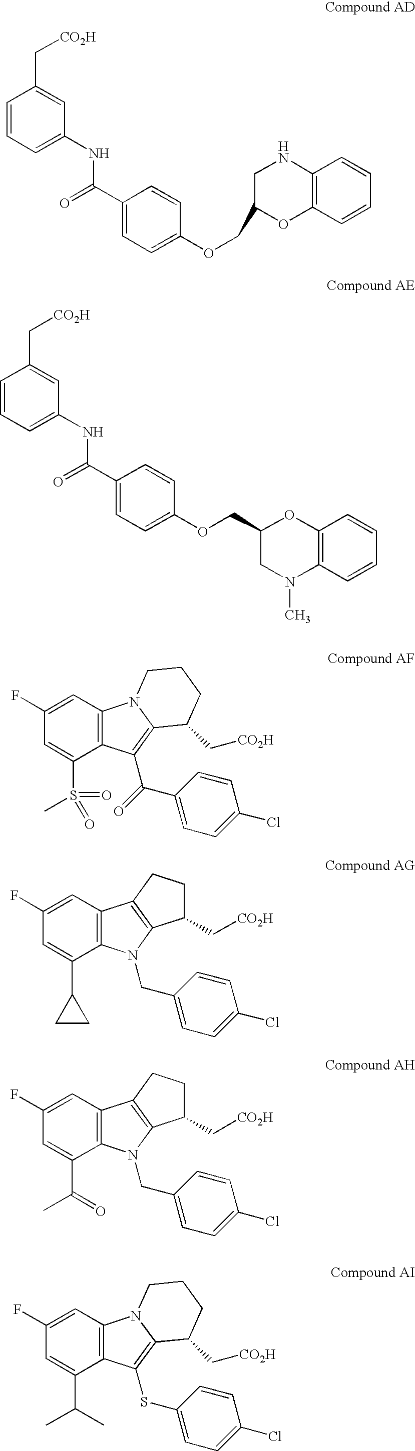

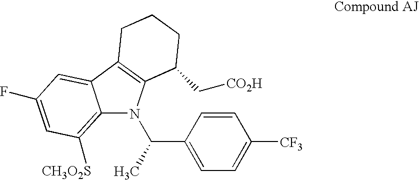

as well as the pharmaceutically acceptable salts and solvates thereof.

The compound of formula I or a pharmaceutically acceptable salt or solvate thereof and the DP antagonist can be administered together or sequentially in single or multiple daily doses, e.g., bid, tid or qid, without departing from the invention. If sustained release is desired, such as a sustained release product showing a release profile that extends beyond 24 hours, dosages may be administered every other day. However, single daily doses are preferred. Likewise, morning or evening dosages can be utilized.

### Salts and Solvates

Salts and solvates of the compounds of formula I are also included in the present invention, and numerous pharmaceutically acceptable salts and solvates of nicotinic acid are useful in this regard. Alkali metal salts, in particular, sodium and potassium, form salts that are useful as described herein. Likewise alkaline earth metals, in particular, calcium and magnesium, form salts that are useful as described herein. Various salts of amines, such as ammonium and substituted ammonium compounds also form salts that are useful as described herein. Similarly, solvated forms of the compounds of formula I are useful within the present invention. Examples include the hemihydrate, mono-, di-, tri- and sesquihydrate.

The compounds of the invention also include esters or ester prodrugs that are pharmaceutically acceptable, as well as those that are metabolically labile. Metabolically labile esters include C₁₋₄ alkyl esters, preferably the ethyl ester. Many prodrug strategies are known to those skilled in the art. One such strategy involves engineered amino acid anhydrides possessing pendant nucleophiles, such as lysine, which can cyclize upon themselves, liberating the free acid. Similarly, acetone-ketal diesters, which can break down to acetone, an acid and the active acid, can be used.

The compounds used in the present invention can be administered via any conventional route of administration. The preferred route of administration is oral.

### Pharmaceutical Compositions

The pharmaceutical compositions described herein are generally comprised of a compound of formula I or a pharmaceutically acceptable salt or solvate thereof, in combination with a pharmaceutically acceptable carrier.

Examples of suitable oral compositions include tablets, capsules, troches, lozenges, suspensions, dispersible powders or granules, emulsions, syrups and elixirs. Examples of carrier ingredients include diluents, binders, disintegrants, lubricants, sweeteners, flavors, colorants, preservatives, and the like. Examples of diluents include, for example, calcium carbonate, sodium carbonate, lactose, calcium phosphate and sodium phosphate. Examples of granulating and disintegrants include corn starch and alginic acid. Examples of binding agents include starch, gelatin and acacia. Examples of lubricants include magnesium stearate, calcium stearate, stearic acid and talc. The tablets may be uncoated or coated by known techniques. Such coatings may delay disintegration and thus, absorption in the gastrointestinal tract and thereby provide a sustained action over a longer period.

In one embodiment, a representative pharmaceutical composition is described in the form of a tablet comprising about 1 mg to about 100 mg of a compound of formula I or a pharmaceutically acceptable salt or solvate thereof in combination with a pharmaceutically acceptable carrier.

In another embodiment of the invention, a compound of formula I or a pharmaceutically acceptable salt or solvate thereof is combined with another therapeutic agent and the carrier to form a fixed combination product. This fixed combination product may be a tablet or capsule for oral use.

More particularly, in another embodiment of the invention, a compound of formula I or a pharmaceutically acceptable salt or solvate thereof (about 1 to about 1000 mg) and the second therapeutic agent (about 1 to about 500 mg) are combined with the pharmaceutically acceptable carrier, providing a tablet or capsule for oral use.

Sustained release over a longer period of time may be particularly important in the formulation. A time delay material such as glyceryl monostearate or glyceryl distearate may be employed. The dosage form may also be coated by the techniques described in the U.S. Pat. Nos. 4,256,108; 4,166,452 and 4,265,874 to form osmotic therapeutic tablets for controlled release.

Other controlled release technologies are also available and are included herein. Typical ingredients that are useful to slow the release of nicotinic acid in sustained release tablets include various cellulosic compounds, such as methylcellulose, ethylcellulose, propylcellulose, hydroxypropylcellulose, hydroxyethylcellulose, hydroxypropylmethylcellulose, microcrystalline cellulose, starch and the like. Various natural and synthetic materials are also of use in sustained release formulations. Examples include alginic acid and various alginates, polyvinyl pyrrolidone, tragacanth, locust bean gum, guar gum, gelatin, various long chain alcohols, such as cetyl alcohol and beeswax.

Optionally and of even more interest is a tablet as described above, comprised of a compound of formula I or a pharmaceutically acceptable salt or solvate thereof, and further containing an HMG Co-A reductase inhibitor, such as simvastatin or atorvastatin. This particular embodiment optionally contains the DP antagonist as well.

Typical release time frames for sustained release tablets in accordance with the present invention range from about 1 to as long as about 48 hours, preferably about 4 to about 24 hours, and more preferably about 8 to about 16 hours.

Hard gelatin capsules constitute another solid dosage form for oral use. Such capsules similarly include the active ingredients mixed with carrier materials as described above. Soft gelatin capsules include the active ingredients mixed with water-miscible solvents such as propylene glycol, PEG and ethanol, or an oil such as peanut oil, liquid paraffin or olive oil.

Aqueous suspensions are also contemplated as containing the active material in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, tragacanth and acacia; dispersing or wetting agents, e.g., lecithin; preservatives, e.g., ethyl, or n-propyl para-hydroxybenzoate, colorants, flavors, sweeteners and the like.

Dispersible powders and granules suitable for preparation of an aqueous suspension by the addition of water provide the active ingredients in admixture with a dispersing or wetting agent, suspending agent and one or more preservatives. Suitable dispersing or wetting agents and suspending agents are exemplified by those already mentioned above.

Syrups and elixirs may also be formulated.

More particularly, a pharmaceutical composition that is of interest is a sustained release tablet that is comprised of a compound of formula I or a pharmaceutically acceptable salt or solvate thereof, and a DP receptor antagonist that is selected from the group consisting of compounds A through AJ in combination with a pharmaceutically acceptable carrier.

Yet another pharmaceutical composition that is of more interest is comprised of a compound of formula I or a pharmaceutically acceptable salt or solvate thereof and a DP antagonist compound selected from the group consisting of compounds A, B, D, E, X, AA, AF, AG, AH, AI and AJ, in combination with a pharmaceutically acceptable carrier.

Yet another pharmaceutical composition that is of more particular interest relates to a sustained release tablet that is comprised of a compound of formula I or a pharmaceutically acceptable salt or solvate thereof, a DP receptor antagonist selected from the group consisting of compounds A, B, D, E, X, AA, AF, AG, AH, AI and AJ, and simvastatin or atorvastatin in combination with a pharmaceutically acceptable carrier.

The term “composition”, in addition to encompassing the pharmaceutical compositions described above, also encompasses any product which results, directly or indirectly, from the combination, complexation or aggregation of any two or more of the ingredients, active or excipient, or from dissociation of one or more of the ingredients, or from other types of reactions or interactions of one or more of the ingredients. Accordingly, the pharmaceutical composition of the present invention encompasses any composition made by admixing or otherwise combining the compounds, any additional active ingredient(s), and the pharmaceutically acceptable excipients.

Another aspect of the invention relates to the use of a compound of formula I or a pharmaceutically acceptable salt or solvate thereof and a DP antagonist in the manufacture of a medicament. This medicament has the uses described herein.

More particularly, another aspect of the invention relates to the use of a compound of formula I or a pharmaceutically acceptable salt or solvate thereof, a DP antagonist and an HMG Co-A reductase inhibitor, such as simvastatin, in the manufacture of a medicament. This medicament has the uses described herein.

Compounds of the present invention have anti-hyperlipidemic activity, causing reductions in LDL-C, triglycerides, apolipoprotein a and total cholesterol, and increases in HDL-C. Consequently, the compounds of the present invention are useful in treating dyslipidemias. The present invention thus relates to the treatment, prevention or reversal of atherosclerosis and the other diseases and conditions described herein, by administering a compound of formula I or a pharmaceutically acceptable salt or solvate in an amount that is effective for treating, preventing or reversing said condition. This is achieved in humans by administering a compound of formula I or a pharmaceutically acceptable salt or solvate thereof in an amount that is effective to treat or prevent said condition, while preventing, reducing or minimizing flushing effects in terms of frequency and/or severity.

One aspect of the invention that is of interest is a method of treating atherosclerosis in a human patient in need of such treatment comprising administering to the patient a compound of formula I or a pharmaceutically acceptable salt or solvate thereof in an amount that is effective for treating atherosclerosis in the absence of substantial flushing.

Another aspect of the invention that is of interest relates to a method of raising serum HDL levels in a human patient in need of such treatment, comprising administering to the patient a compound of formula I or a pharmaceutically acceptable salt or solvate thereof in an amount that is effective for raising serum HDL levels.

Another aspect of the invention that is of interest relates to a method of treating dyslipidemia in a human patient in need of such treatment comprising administering to the patient a compound of formula I or a pharmaceutically acceptable salt or solvate thereof in an amount that is effective for treating dyslipidemia.

Another aspect of the invention that is of interest relates to a method of reducing serum VLDL or LDL levels in a human patient in need of such treatment, comprising administering to the patient a compound of formula I or a pharmaceutically acceptable salt or solvate thereof in an amount that is effective for reducing serum VLDL or LDL levels in the patient in the absence of substantial flushing.

Another aspect of the invention that is of interest relates to a method of reducing serum triglyceride levels in a human patient in need of such treatment, comprising administering to the patient a compound of formula I or a pharmaceutically acceptable salt or solvate thereof in an amount that is effective for reducing serum triglyceride levels.

Another aspect of the invention that is of interest relates to a method of reducing serum Lp(a) levels in a human patient in need of such treatment, comprising administering to the patient a compound of formula I or a pharmaceutically acceptable salt or solvate thereof in an amount that is effective for reducing serum Lp(a) levels. As used herein Lp(a) refers to lipoprotein (a).

Another aspect of the invention that is of interest relates to a method of treating diabetes, and in particular, type 2 diabetes, in a human patient in need of such treatment comprising administering to the patient a compound of formula I or a pharmaceutically acceptable salt or solvate thereof in an amount that is effective for treating diabetes.

Another aspect of the invention that is of interest relates to a method of treating metabolic syndrome in a human patient in need of such treatment comprising administering to the patient a compound of formula I or a pharmaceutically acceptable salt or solvate thereof in an amount that is effective for treating metabolic syndrome.

Another aspect of the invention that is of particular interest relates to a method of treating atherosclerosis, dyslipidemias, diabetes, metabolic syndrome or a related condition in a human patient in need of such treatment, comprising administering to the patient a compound of formula I or a pharmaceutically acceptable salt or solvate thereof and a DP receptor antagonist, said combination being administered in an amount that is effective to treat atherosclerosis, dyslipidemia, diabetes or a related condition in the absence of substantial flushing.

Another aspect of the invention that is of particular interest relates to the methods described above wherein the DP receptor antagonist is selected from the group consisting of compounds A through AJ and the pharmaceutically acceptable salts and solvates thereof.

### Methods of Synthesis for Compounds of Formula I

Compounds of Formula I have been prepared by the following representative reaction schemes. It is understood that similar reagents, conditions or other synthetic approaches to these structure classes are conceivable to one skilled in the art of organic synthesis. Therefore these reaction schemes should not be construed as limiting the scope of the invention. All substituents are as defined above unless indicated otherwise.

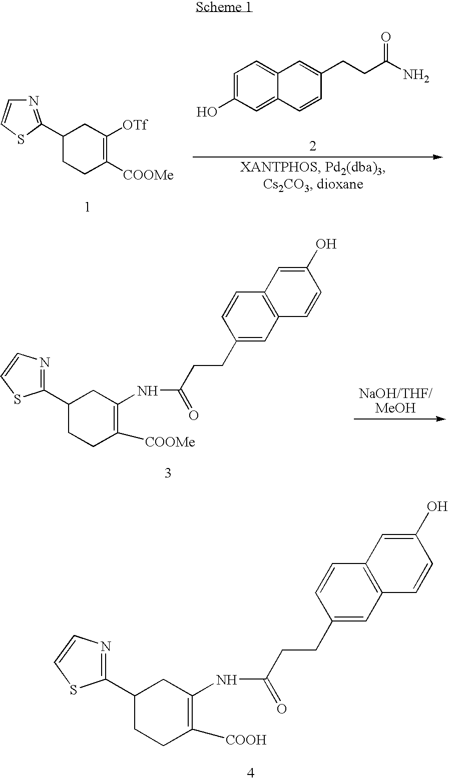

Scheme 1 outlines the preparation of compounds with the structure 4 (see Wallace et al, Organic Letters, 2003, 4749). Thus, treatment of the triflate 1 with a suitable amide like 2 in the presence of a catalyst such as Pd₂(dba)₃, ligand such as XANTPHOS, and a base such as cesium carbonate in a polar solvent such as 1,4-dioxane gives the desired amide 3. The ester can be saponified by methods known to those skilled in the art such as NaOH/THF/MeOH—H₂O providing the desired compound 4.

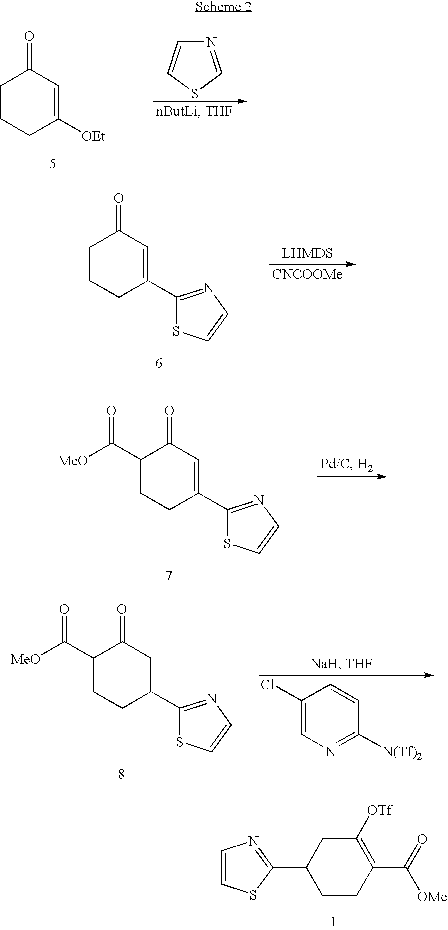

Scheme 2 outlines the preparation of the triflate 1. De-protonation of thiazole can generate an anion for the 1,2-addition to 3 ethoxy-cyclohexenone 5 followed by rearrangement to the beta-substituted en-one 6. Installation of the methyl ester can be accomplished by treatment of 6 with a suitable non-nucleophilic base such as LDA or LHMDS followed by Mander's reagent to give 7 (see Mander et al, Tetrahedron Letters, 1983, 5425). Hydrogenation of the double bond can be achieved using standard conditions such as H₂(g), Pd/C in a suitable polar solvent like methanol or ethanol to give 8. Finally, the enol-triflate 1 can be prepared by treatment of 8 with a suitable base such as sodium hydride followed by a triflating reagent such as Comin's reagent in a solvent like THF (see Comins et al, Tetrahedron Letters, 1992, 6299) to give the desired product.

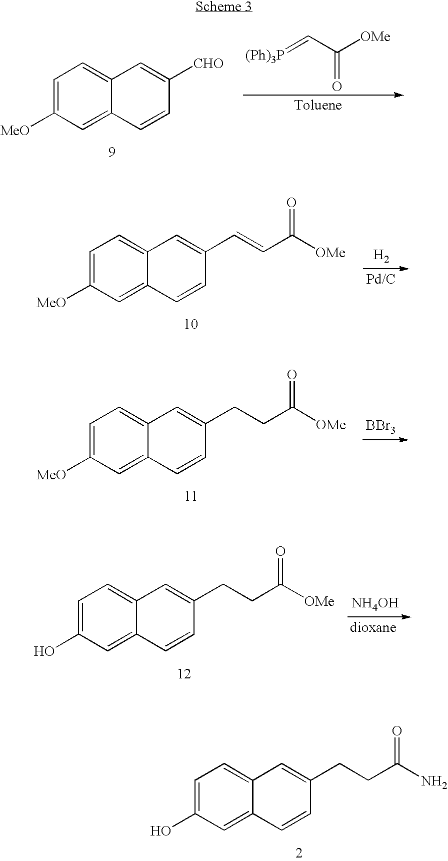

The synthesis of the amide 2 is outlined in Scheme 3. Thus, 6-methoxy-2-naphthaldehyde 9 can be treated with a suitable ylide such as methyl(triphenylphosphoranylidene)acetate in a non-polar solvent such as toluene or xylenes under refluxing conditions to give the desired olefin 10. Hydrogenation of the double bond can be accomplished using standard conditions such as H₂(g), Pd/C in a suitable polar solvent like methanol or ethanol to give 11. De-methylation of the phenol can be accomplished with boron tribromide at low temperature to give 12. Finally, treatment of the ester with ammonium hydroxide solution in dioxane gives the desired carboxamide product 2.

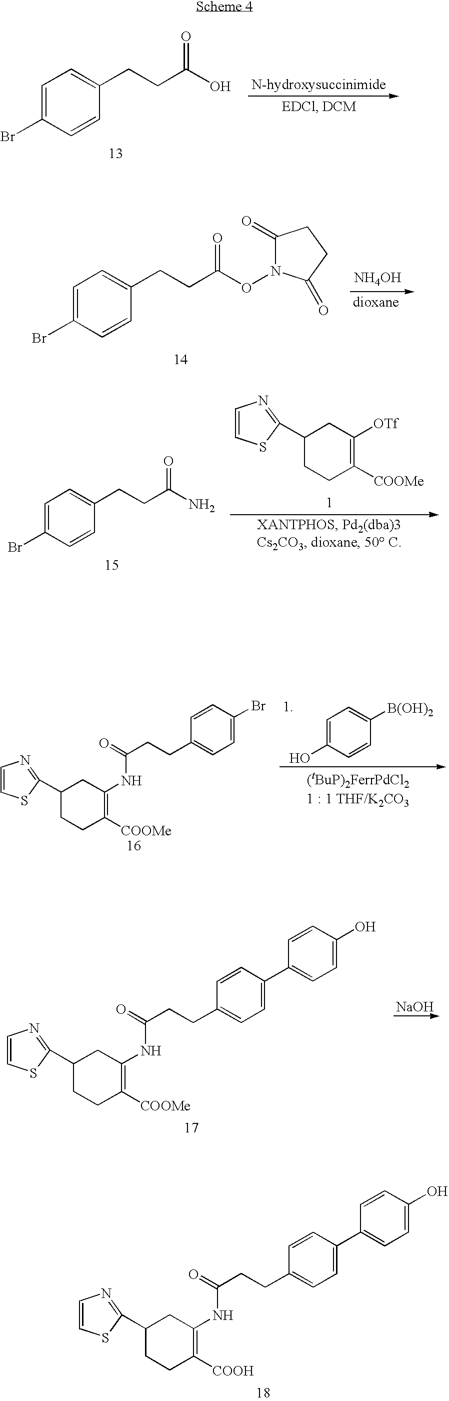

Scheme 4 outlines the strategy used to synthesize compounds of the structure 18. Coupling commercially available 3-(4-bromophenyl) propionic acid 13 with N-hydroxy succinimide using a suitable coupling reagent such as EDCI gives the ester 14. This material can be converted to the amide 15 by treatment with ammonium hydroxide. Coupling with the triflate 1 is accomplished using conditions described in Scheme 1. The bromide 16 can be converted to 17 via a Suzuki reaction with a suitable boronic acid such as 4-hydroxy phenyl boronic acid in the presence of a catalyst such as Bis-tert-butyl-ferrocene palladium dichloride. Finally, the methyl ester can be saponified by methods known to those skilled in the art providing compounds of the structure 18.

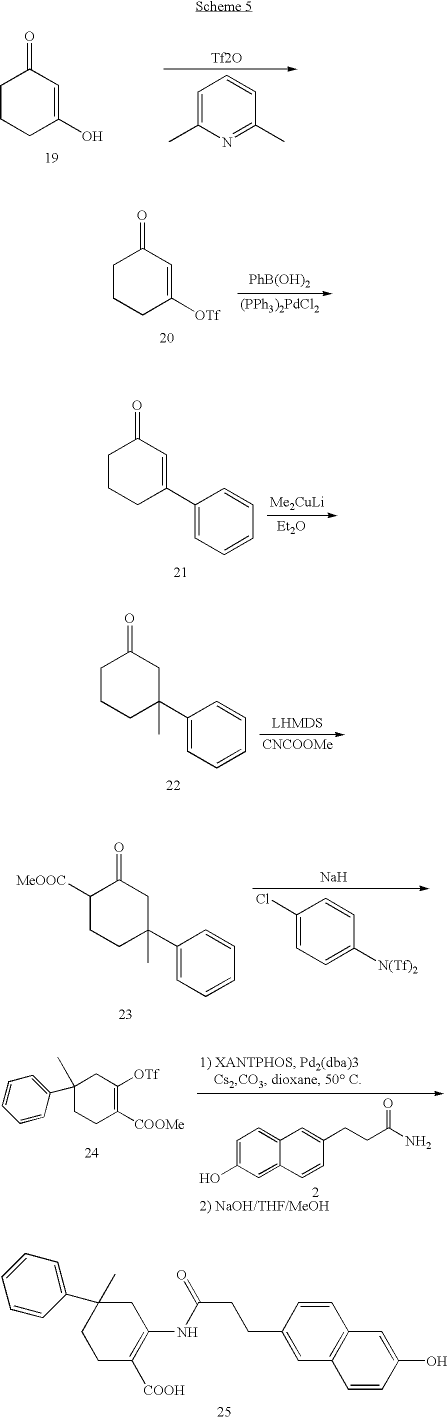

Scheme 5 outlines the strategy used to synthesize compounds of the structure 25. The enol-triflate 20 can be prepared by treatment of cyclohexane-1,3-dione 19 with triflic anhydride and 2,6-lutidine. The triflate 20 can be converted to the 3-substituted enone 21 via a standard Suzuki reaction with a suitable boronic acid such as phenyl boronic acid in the presence of a catalyst such as dichlorobis-(triphenyl phosphine) palladium. The enone 21 can be converted to the 3,3-disubstituted ketone 22 via a methyl cuprate addition to the enone using standard conditions. Installation of the methyl ester can be accomplished by treatment of 22 with a suitable non-nucleophilic base such as LDA or LHMDS followed by Mander's reagent to give 23. This intermediate can be converted to the vinyl triflate 24 using conditions described in Scheme 2. Finally, the triflate 24 is converted to the desired product 25 using the coupling and saponification procedures described earlier (Scheme 1).

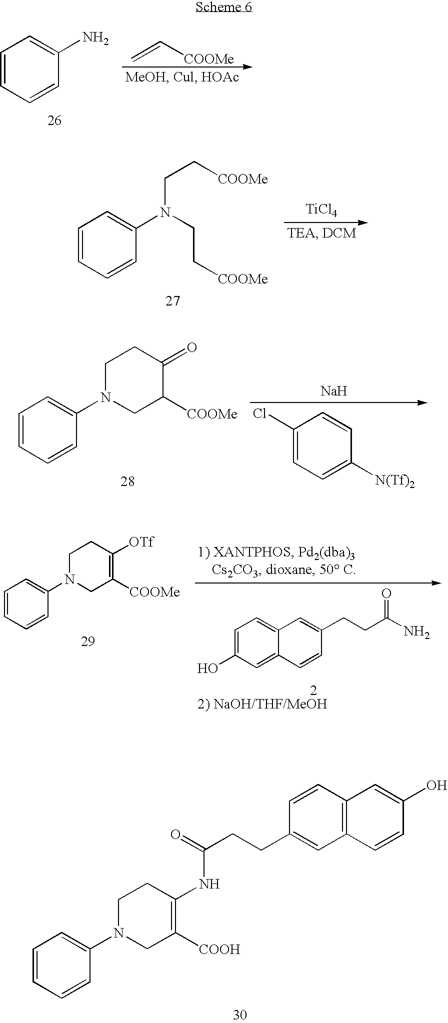

Compounds with the structure 30 can be prepared using the strategy outlined in Scheme 6. 3-Carbomethoxy-4-phenyl-piperidone 28 can be prepared using the procedure described by Deshmukh, et al Synthetic Communications, 1995, 177. Thus, treatment of an aniline 26 with excess methyl acrylate in methanol in the presence of copper iodide and acetic acid gives the N-substituted di(β-carbomethoxyethyl) amine 27. Dieckmann cyclization of 27 to 28 can be accomplished with titanium tetrachloride in dichloromethane in the presence of triethyl amine. This material can be converted to the vinyl triflate 29 using the conditions described in Scheme 2. Finally, the triflate 29 can be converted to the desired product 30 using the coupling and saponification procedures described earlier (Scheme 1).

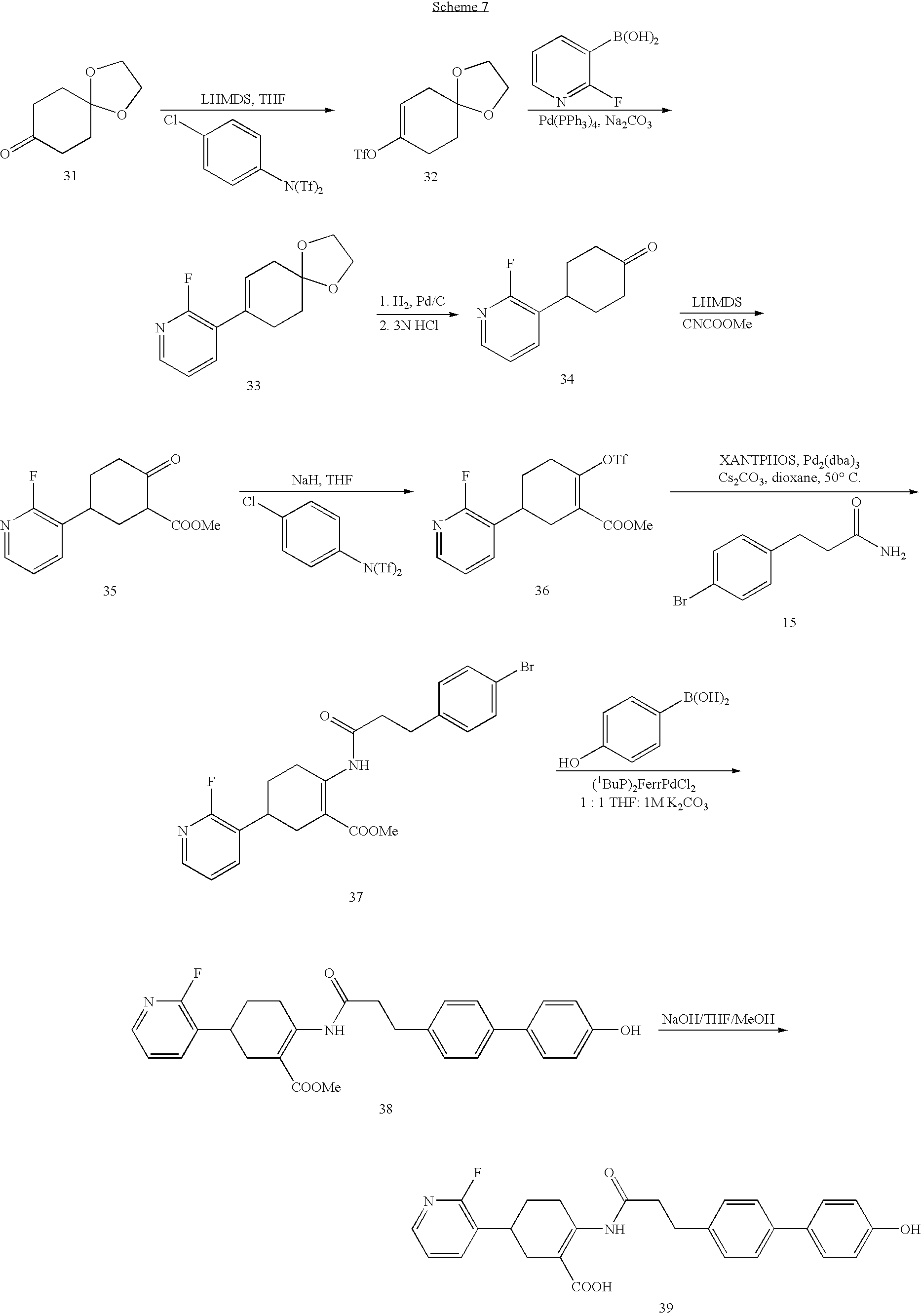

Scheme 7 outlines the strategy used for the synthesis of compounds of the structure 39. Thus, cyclohexane-1,4-dione mono-ketal 31 can be converted to the triflate 32 using a suitable base like LDA or LHMDS and a triflating agent like Comin's reagent. The vinyl triflate 32 can be converted to the substituted olefin 33 via a standard Suzuki reaction with a suitable boronic acid such as 2-fluoro-3-pyridyl phenyl boronic acid in the presence of a catalyst such as dichlorobis-(triphenyl phosphine) palladium or tetrakis triphenyl phosphine palladium (0). Hydrogenation of the double bond can be achieved using standard conditions such as H₂(g), Pd/C in a suitable polar solvent like methanol or ethanol followed by the removal of ketal protecting group using standard aqueous acid catalyzed conditions to give the ketone 34. Acylation of the ketone 34 is accomplished using a suitable base like LDA or LHMDS and Mander's reagent to obtain 35. This material can be converted to the vinyl triflate 36 using the conditions described in Scheme 2. Finally, the triflate 36 can be converted to the desired product 39 using the coupling and saponification procedures described earlier (Scheme 4).

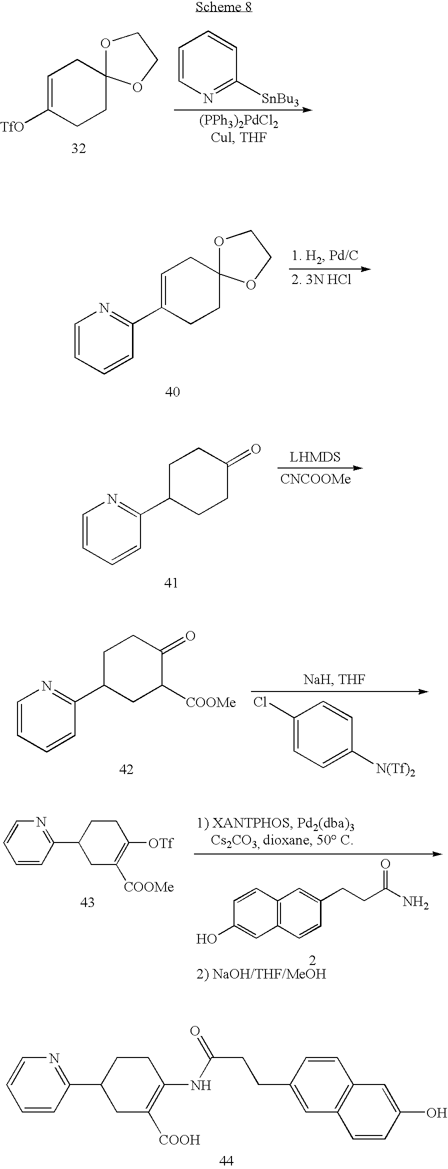

Scheme 8 outlines the route used to synthesize compounds of the structure 44. Thus, the vinyl triflate 32 is coupled to 2-pyridyl tri-n-butyl stannane via a standard Stille procedure in the presence of copper iodide or lithium chloride and a catalyst such as dichlorobis-(triphenyl phosphine) palladium or tetrakis triphenyl phosphine palladium (0) to give 40. This material is converted to the triflate 43 using the route outlined in Scheme 7. Finally, the triflate 43 can be converted to the desired product 44 using the coupling and saponification procedures described earlier (Scheme 1).

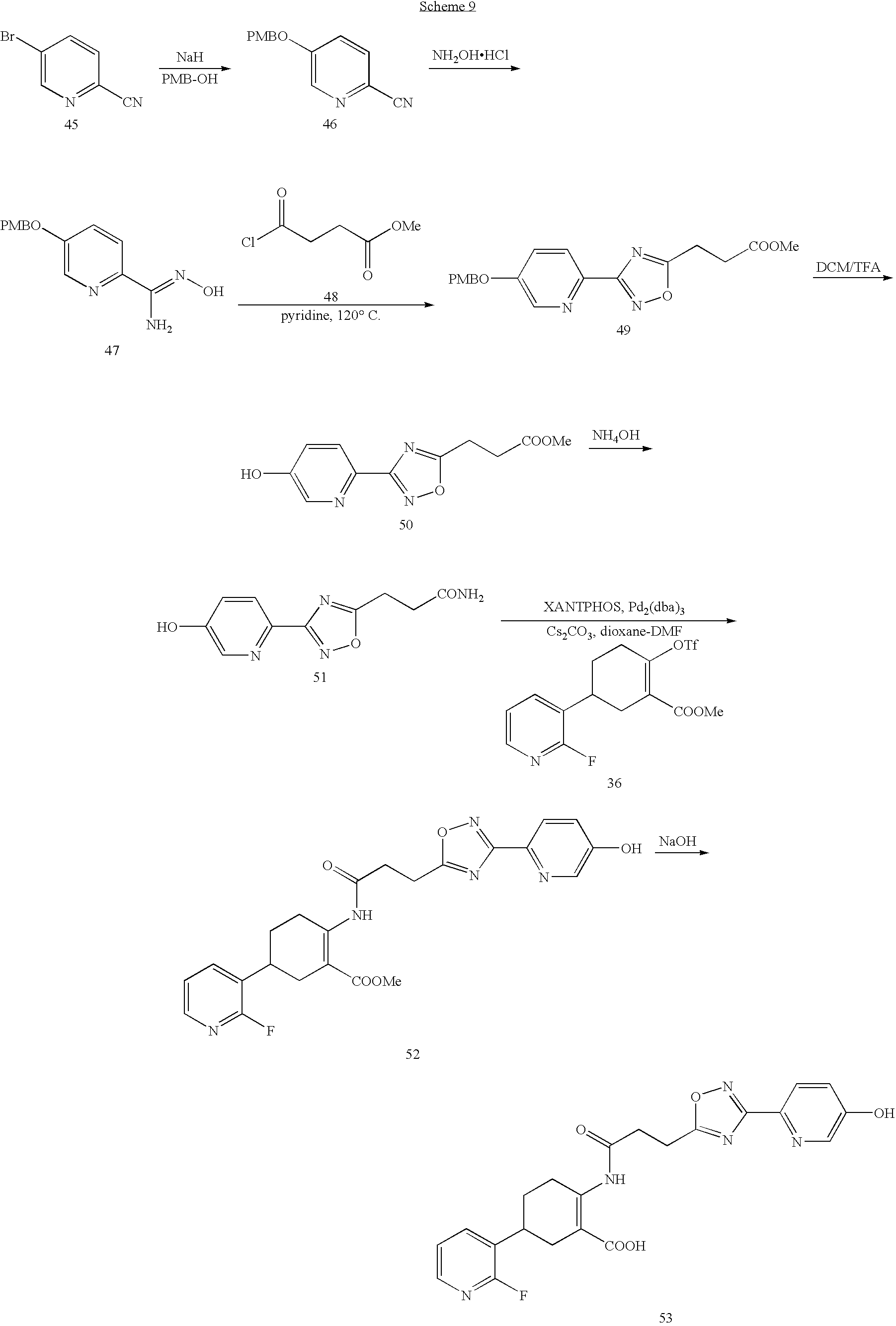

Scheme 9 outlines the strategy used for the synthesis of compounds of the structure 53. Thus, 5-bromo-2-cyano pyridine can be treated with sodium hydride and 4-methoxy benzyl alcohol to give the intermediate 46. This material can be converted to the intermediate 47 by treatment with hydroxylamine hydrochloride in the presence of a suitable base such as NaOH. Acylation followed by cyclization to the oxadiazole 49 can be accomplished by treatment of the intermediate 47 with the commercially available acid chloride 48 in a suitable solvent such as pyridine followed by heating to reflux. The removal of the PMB protecting group can be accomplished using standard methods known to one skilled in the art such as TFA/DCM. Treatment of the ester 50 with ammonium hydroxide solution in dioxane gives the desired carboxamide 51. Finally, the triflate 36 (Scheme 7) can be converted to the desired product 53 using coupling and saponification conditions described earlier (Scheme 1).

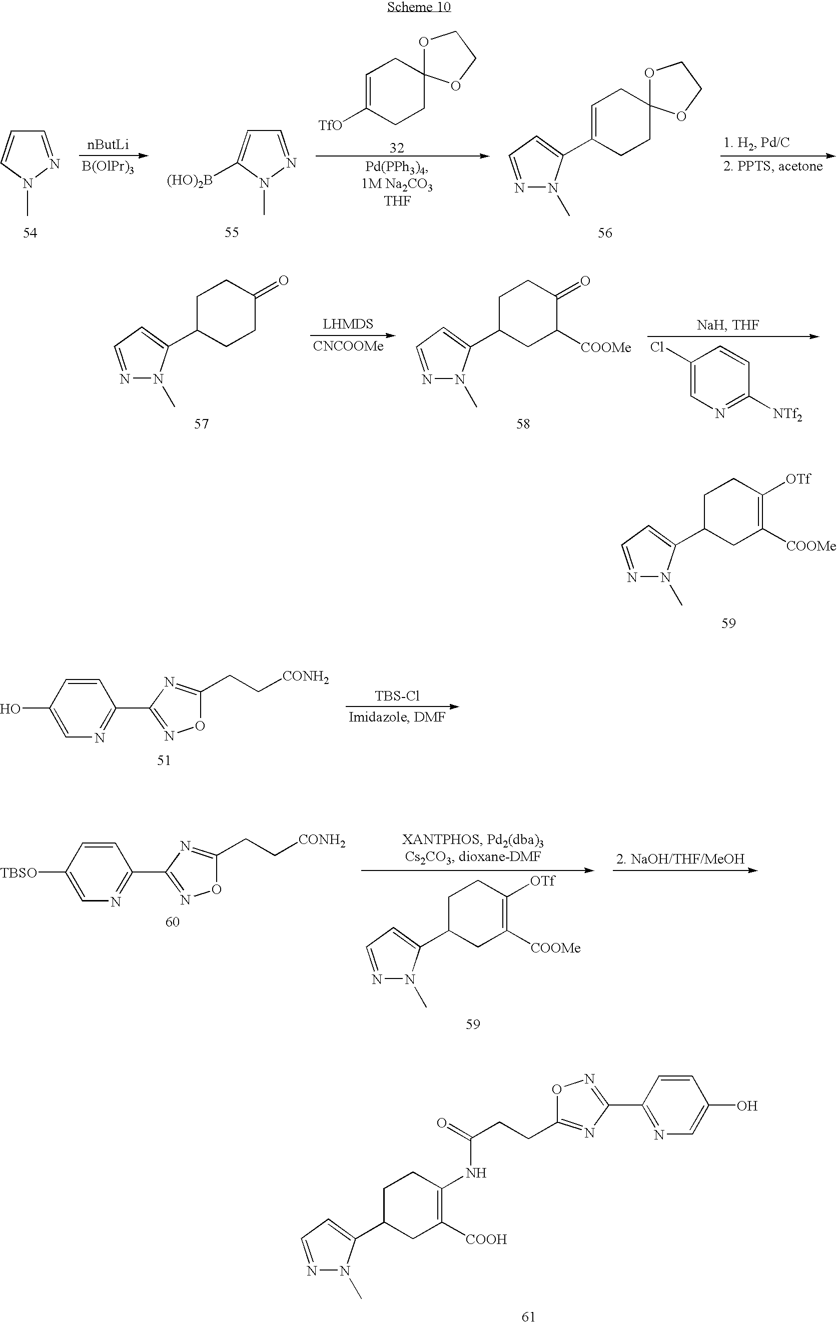

Scheme 10 outlines the strategy used for the synthesis of compounds of the structure 61. Thus, treatment of methylpyrazole 54 with nButyl lithium and triisopropyl borate followed by an acidic work up gives the desired boronic acid 55. The boronic acid is coupled to the triflate 32 via a standard Suzuki reaction and elaborated to the desired vinyl triflate 59 following procedures described earlier (Scheme 7). Compound 51 can be protected with the TBS group to give 60 using methods known to one skilled in the art such as TBS-Cl and a suitable base like imidazole. Finally, the amide 60 can be coupled to the triflate 59 using the conditions described earlier followed by saponification of the methyl ester and deprotection to give the desired product 61 (Scheme 1).

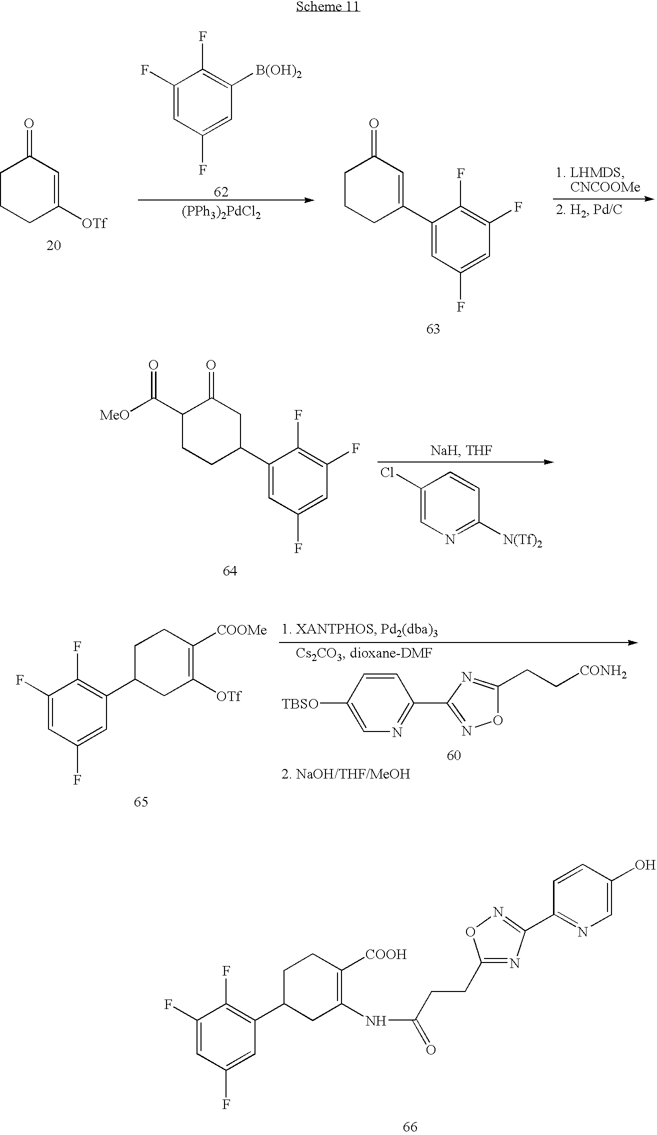

Scheme 11 outlines the synthetic route used to access compounds with the structure 66. The enol-triflate 20 (Scheme 5) can be converted to the 3-(2,3,5-trifluorophenyl) en-one 63 via a standard Suzuki reaction with 2,3,5-trifluorophenyl boronic acid 62 in the presence of a catalyst such as dichlorobis-(triphenylphosphine)palladium. The en-one 63 can be acylated in the presence of a suitable base such as LDA or LHMDS with Mander's reagent followed by hydrogenation using Pd/C as catalyst to give the desired keto-ester 64. The keto-ester 64 is converted to the vinyl triflate 65 using conditions described in Scheme 2. Finally, the triflate 65 is converted to the desired product 66 using the coupling and saponification procedures described earlier (Scheme 1).

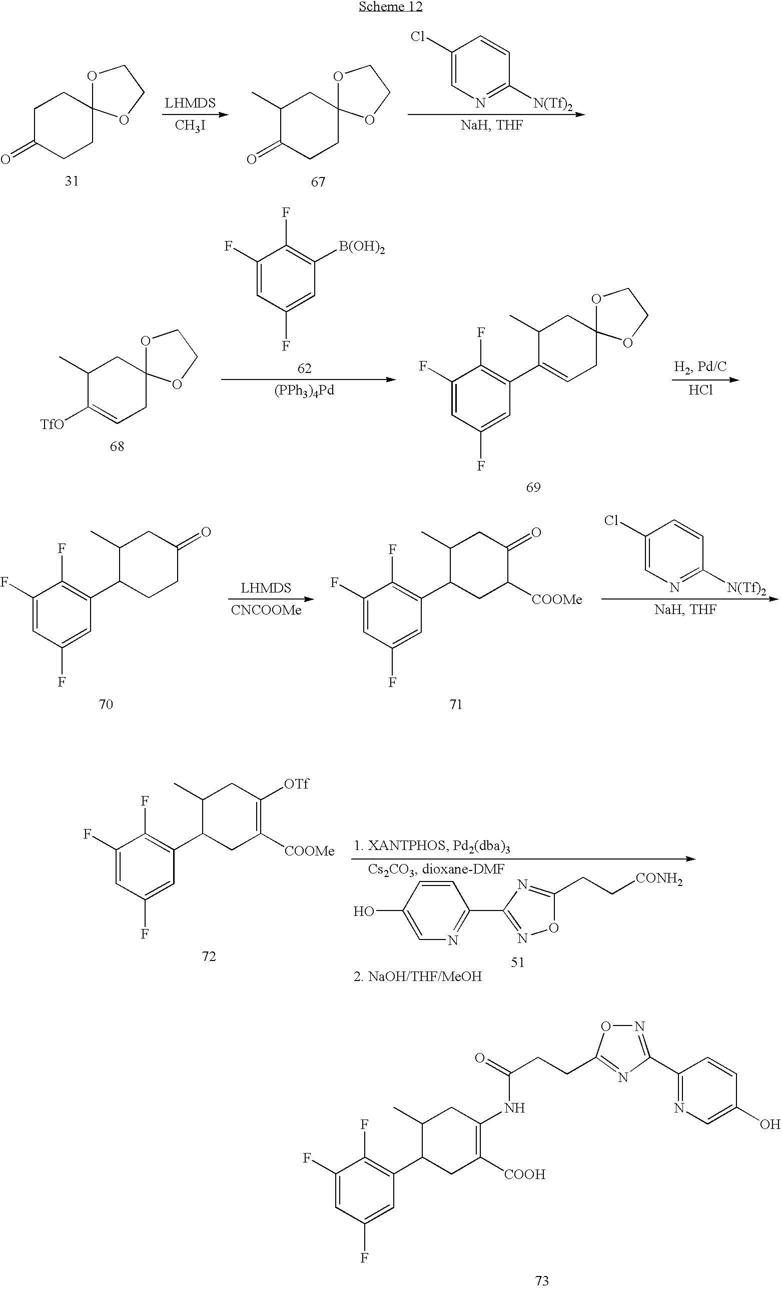

Scheme 12 outlines the preparation of compounds with the structure 73. Cyclohexane-1,4-dione mono-ketal 31 can be alkylated with methyl iodide in the presence of a suitable base such as LHMDS to give 67 using the procedure described by Danishefsky, et al J. Am. Chem. Soc. 2004, 126, 14358. The ketone 67 is converted to the triflate 68 using a suitable base like LDA or LHMDS and a triflating agent like Comin's reagent. The vinyl triflate 68 can be converted to the substituted olefin 69 via a standard Suzuki reaction with a suitable boronic acid such as 2,3,5 trifluoro phenyl boronic acid in the presence of a catalyst such as dichloro-(triphenyl phosphine) palladium or tetrakis triphenyl phosphine palladium(0). Hydrogenation of the double bond can be achieved using standard conditions such as H₂(g), Pd/C in a suitable polar solvent like methanol or ethanol followed by removal of the ketal protecting group under standard aqueous acid catalyzed conditions to give the ketone 70. Acylation of the ketone 70 is accomplished using a suitable base like LDA or LHMDS and Mander's reagent to obtain 71. This material can be converted to the vinyl trifate 72 using the conditions described in Scheme 2. Finally, the triflate 72 can be converted to the desired product 73 using the coupling and saponification procedures described earlier (Scheme 1).

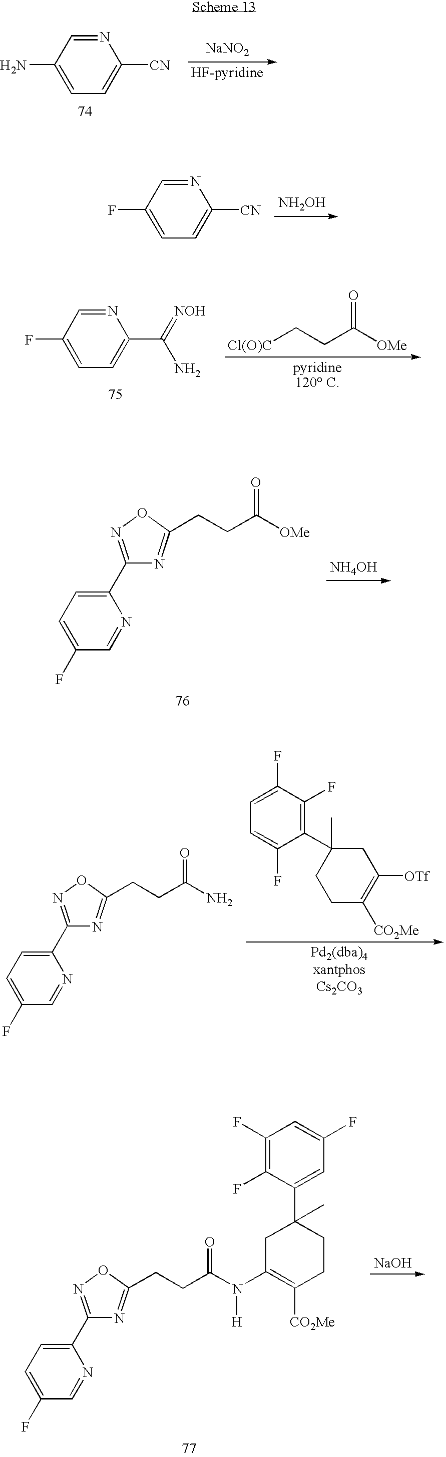

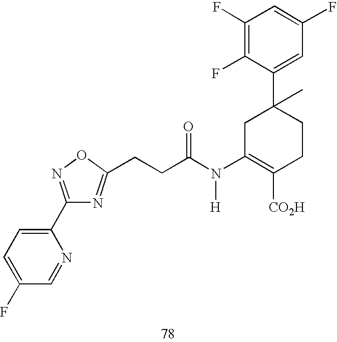

Scheme 13 illustrates the preparation of compounds related to structure 78. For example, the cyanoaminopyridine 74 can be fluorinated and treated with hydroxylamine to give 75 under standard conditions known to those skilled in the art. This intermediate can then be cyclized to form an oxadiazole 76, converted to a carboxamide, and then coupled with a vinyl triflate (Scheme 5) to afford 77. Upon saponification, the desired product 78 may be obtained.

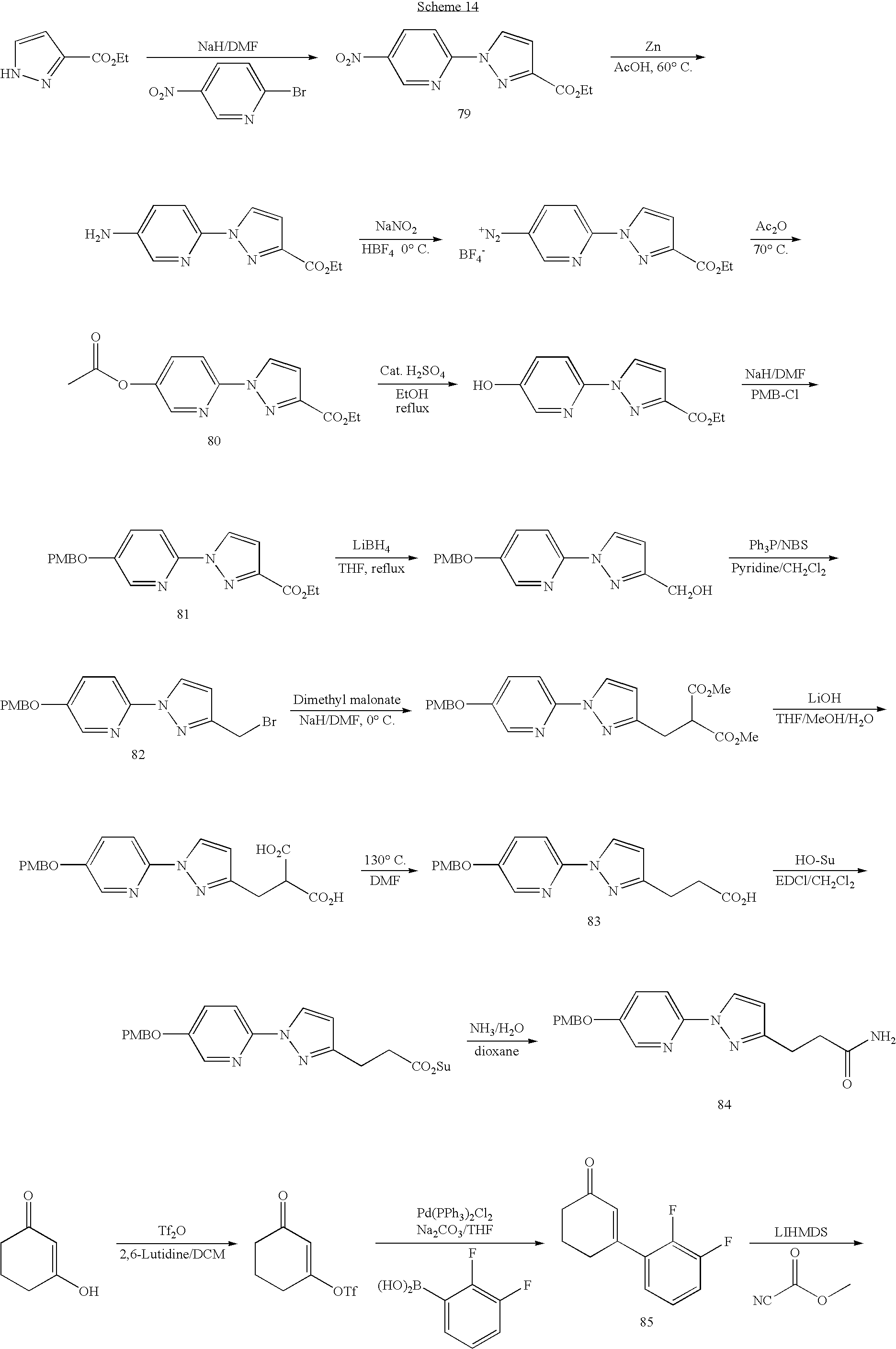

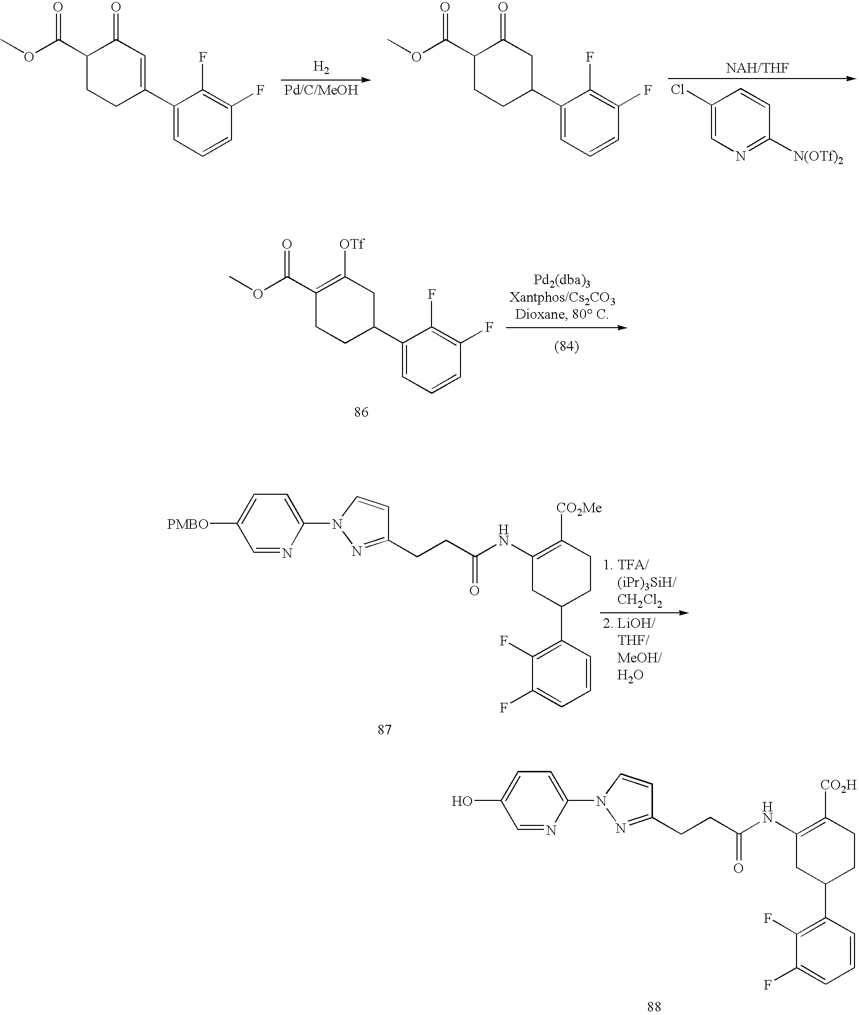

Scheme 14 illustrates the preparation of compounds related to structure 88. For example, ethyl 3-pyrazole carboxylate can be arylated with an electron deficient bromopyridine to form 79. The nitro functionality can be reduced, the amine converted to the diazo intermediate, and trapped with anhydride to form 80. Hydrolysis of the acetate and protection of the alcohol provides 81. Reduction of the ester and bromination can provide the electrophile 82. Subsequent displacement with malonate, hydrolysis and decarboxylation provides the acid 83. This acid may then be converted to its carboxamide 84. In parallel, 1,3-cyclohexanedione can be converted to its triflate, arylated to give 85, carboxylated with Mander's reagent, hydrogenated, and triflated to form intermediate 86. This triflate 86 can be coupled with 84 to form 87. Upon bis-deprotection under conditions known to those skilled in the art, 88 may be obtained.

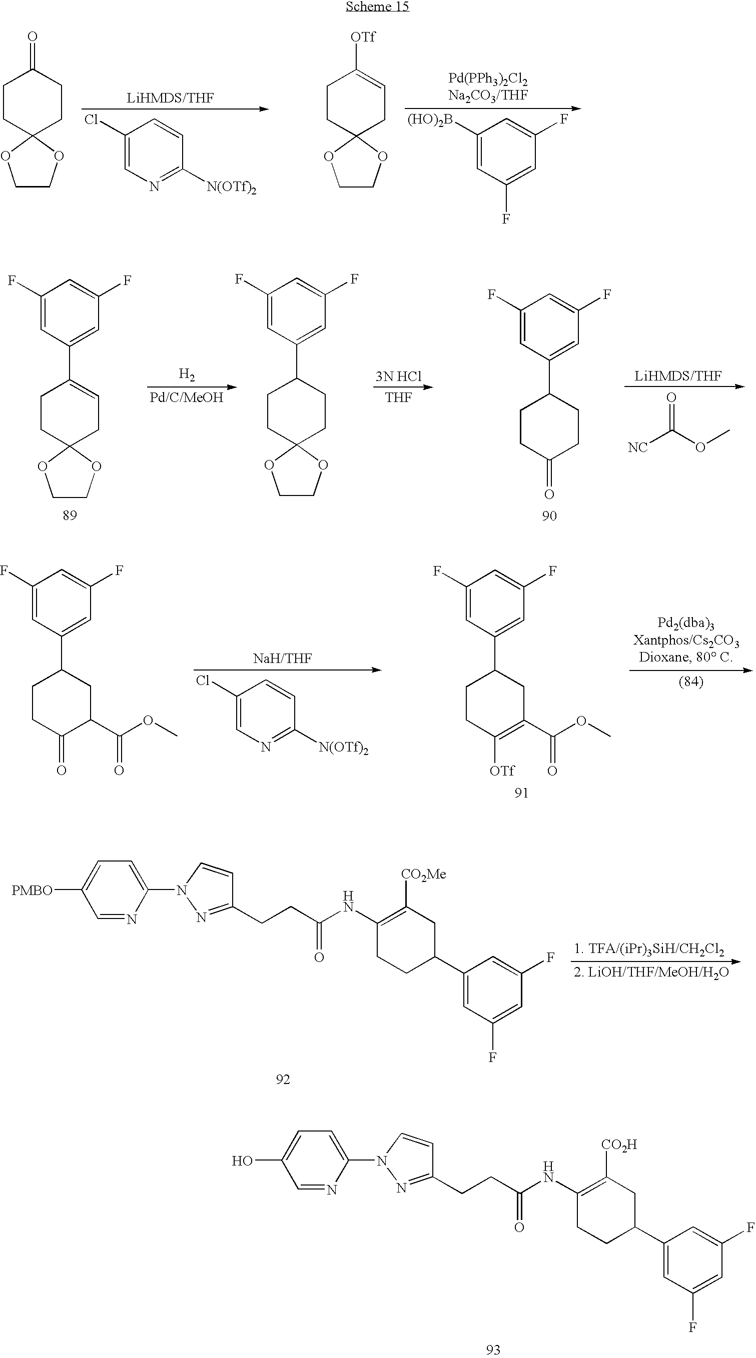

Scheme 15 illustrates the preparation of compounds related to structure 93. For instance, 1,4-cyclohexanedione monoketal can be triflated and arylated to form 89. Hydrogenation of the double bond and hydrolysis of the ketal can provide 90. This intermediate can be carboxylated with Mander's reagent as above, and triflation then provides intermediate 91. Similar coupling conditions as shown in previous schemes can unite intermediates such as 91 and 84 to provide 92, which upon bis-deprotection under conditions known to those skilled in the art, generates compounds such as 93.

The various organic group transformations and protecting groups utilized herein can be performed by a number of procedures other than those described above. References for other synthetic procedures that can be utilized for the preparation of intermediates or compounds disclosed herein can be found in, for example, M. B. Smith, J. March Advanced Organic Chemistry, 5^{th} Edition, Wiley-Interscience (2001); R. C. Larock Comprehensive Organic Transformations, A Guide to Functional Group Preparations, 2^{nd} Edition, VCH Publishers, Inc. (1999); T. L. Gilchrist Heterocyclic Chemistry, 3^{rd} Edition, Addison Wesley Longman Ltd. (1997); J. A. Joule, K. Mills, G. F. Smith Heterocyclic Chemistry, 3^{rd} Edition, Stanley Thornes Ltd. (1998); G. R. Newkome, W. W. Paudler Contempory Heterocyclic Chemistry, John Wiley and Sons (1982); or Wuts, P. G. M.; Greene, T. W.; Protective Groups in Organic Synthesis, 3^{rd} Edition, John Wiley and Sons, (1999), all six incorporated herein by reference in their entirety.

### REPRESENTATIVE EXAMPLES

The following examples are provided to more fully illustrate the present invention, and shall not be construed as limiting the scope in any manner. Unless stated otherwise:

(i) all operations were carried out at room or ambient temperature, that is, at a temperature in the range 18-25° C.;

(ii) evaporation of solvent was carried out using a rotary evaporator under reduced pressure (4.5-30 mmHg) with a bath temperature of up to 50° C.;

(iii) the course of reactions was followed by thin layer chromatography (TLC) and/or tandem high performance liquid chromatography (HPLC) followed by mass spectroscopy (MS), herein termed LCMS, and any reaction times are given for illustration only;

(iv) yields, if given, are for illustration only;

(v) the structure of all final compounds was assured by at least one of the following techniques: MS or proton nuclear magnetic resonance (1H NMR) spectrometry, and the purity was assured by at least one of the following techniques: TLC or HPLC;

(vi) 1H NMR spectra were recorded on either a Varian Unity or a Varian Inova instrument at 500 or 600 MHz using the indicated solvent; when line-listed, NMR data is in the form of delta values for major diagnostic protons, given in parts per million (ppm) relative to residual solvent peaks (multiplicity and number of hydrogens); conventional abbreviations used for signal shape are: s. singlet; d. doublet (apparent); t. triplet (apparent); m. multiplet; br. broad; etc.;

(vii) MS data were recorded on a Waters Micromass unit, interfaced with a Hewlett-Packard (Agilent 1100) HPLC instrument, and operating on MassLynx/OpenLynx software; electrospray ionization was used with positive (ES+) or negative ion (ES−) detection; the method for LCMS ES+ was 1-2 mL/min, 10-95% B linear gradient over 5.5 min (B=0.05% TFA-acetonitrile, A=0.05% TFA-water), and the method for LCMS ES− was 1-2 mL/min, 10-95% B linear gradient over 5.5 min (B=0.1% formic acid-acetonitrile, A=0.1% formic acid-water), Waters XTerra C18−3.5 um−50×3.0 mmID and diode array detection;

(viii) automated purification of compounds by preparative reverse phase HPLC was performed on a Gilson system using a YMC-Pack Pro C18 column (150×20 mm i.d.) eluting at 20 mL/min with 0-50% acetonitrile in water (0.1% TFA);

(ix) column chromatography was carried out on a glass silica gel column using Kieselgel 60, 0.063-0.200 mm (Merck), or a Biotage cartridge system;

(x) chemical symbols have their usual meanings; the following abbreviations have also been used v (volume), w (weight), b.p. (boiling point), m.p. (melting point), L (litre(s)), mL (millilitres), g (gram(s)), mg (milligrams(s)), mol (moles), mmol (millimoles), eq or equiv (equivalent(s)), IC50 (molar concentration which results in 50% of maximum possible inhibition), EC50 (molar concentration which results in 50% of maximum possible efficacy), uM (micromolar), nM (nanomolar);

(xi) definitions of acronyms are as follows:

BBr₃ is boron tribromide;

DIBALH is diisobutyl aluminum hydride;

TBSOTF is t-butyl dimethyl silyl trifluoromethane sulfonate;

TBS Chloride is t-butyl dimethyl silyl chloride;

THF is tetrahydrofuran;

DMF is dimethylformamide;

DCM is dichloromethane (methylene chloride);

OTf is triflate;

Pd(PPh₃)₄ is tetrakis triphenylphosphine palladium (0);

PMBO is para-methoxybenzyloxy;

PPTS is pyridinium para-toluene sulfonic acid;

TFA is trifluoroacetic acid;

TBAF is tetrabutylammonium fluoride;

LDA is lithium diisopropyl amide;

LHMDS is lithium bis(trimethylsilyl)amide;

DMAP is 4-dimethyl amino pyridine; and

DMSO is dimethyl sulfoxide.

### Example 1

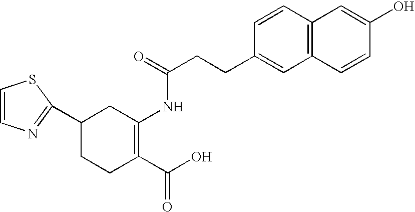

### Step A

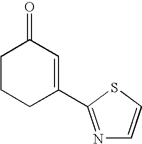

To a solution of thiazole (1 mL, 14.02 mmol) in anhydrous THF (40 mL) cooled to −78° C. was added nButLi (9.35 mL, 14.96 mmol, 1.6 M in hexanes). After 20 minutes, 3-ethoxy-cyclohexene-one (1.26 mL, 9.35 mmol) was added. The reaction was warmed to room temperature and stirred for 30 minutes. The reaction was quenched with 1N HCl (30 mL). The resulting mixture was stirred at room temperature for 16 hours. The layers were separated and the aqueous layer was extracted with ethyl acetate (3×). The combined organic layers were washed with brine, dried over anhydrous sodium sulfate, filtered and concentrated in vacuo. The residue was purified by flash chromatography using 20% ethyl acetate-hexanes to give the desired product as a yellow-brown solid.

### Step B

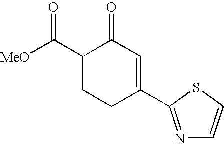

To a solution of the intermediate from step A (1.2 g, 6.69 mmol) in anhydrous THF (50 mL) cooled to −78° C. was added LHMDS (7.36 mL, 7.36 mmol, 1.0 M in THF). After 20 minutes, methyl cyanoformate (0.63 mL, 8.02 mmol) was added. The reaction was slowly warmed to −20° C. and quenched with 1N HCl. The resulting mixture was extracted with ethyl acetate (3×). The combined organic layers were washed with brine, dried over anhydrous sodium sulfate, filtered and concentrated in vacuo. The residue was purified by flash chromatography using 20% ethyl acetate-hexanes to give the desired product as yellow oil.

### Step C

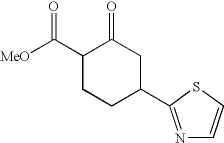

To a solution of the intermediate from step B (1.2 g, 5.06 mmol) in ethyl acetate (20 mL) was added methanol (2 mL) followed by Pd(OH)₂ (0.1 g). The resulting mixture was stirred under a hydrogen balloon for 18 hours. The reaction mixture was filtered through celite and the residue washed with methanol. The filtrate was concentrated in vacuo and purified by flash chromatography using 15% ethyl acetate-hexanes to give the desired product as an oil.

### Step D

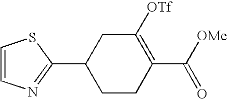

To a solution of the intermediate from step C (0.425 g, 1.77 mmol) in anhydrous THF (20 mL) cooled to 0° C. was added sodium hydride (0.106 g, 2.65 mmol, 60% by weight). After 30 minutes, 2-[N,N-Bis(trifluoromethylsulfonyl)amino]-5-chloropyridine (0.83 g, 2.12 mmol) was added. The reaction mixture was stirred at room temperature for two hours and then quenched with saturated ammonium chloride solution. The resulting mixture was extracted with ethyl acetate (3×). The combined organic layers were washed with brine, dried over anhydrous sodium sulfate, filtered and concentrated in vacuo. The residue was purified by flash chromatography using 15% ethyl acetate-hexanes to give the desired product as colorless oil.

### Step E

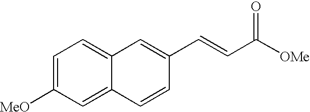

To a solution of 6-methoxy-2-naphthaldehyde (3.72 g, 20.0 mmol) in toluene (40 mL) placed in a pressure vessel was added methyl(triphenylphosphoranylidene)acetate (6.7 g, 20 mmol). The resulting mixture was refluxed at 120° C. for 18 hours. The reaction mixture was concentrated in vacuo and purified using Biotage flash 40M column with 15% ethyl acetate-hexanes as the eluant.

### Step F

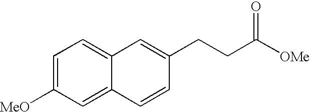

To a solution of the intermediate from step E (4.64 g, 19.14 mmol) in 1:1 dichloromethane-methanol (100 mL) was added Pd/C. The resulting mixture stirred under a H₂ balloon at room temperature for 18 hours. The reaction mixture was filtered through celite and concentrated in vacuo to give the desired compound as a white solid.

### Step G

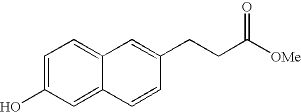

To a solution of the intermediate from Step F (3.0 g, 12.3 mmol) in DCM (80 mL) cooled to 0° C. was added BBr₃ (61.5 mL, 1.0M in DCM). After 30 minutes, the reaction was quenched with methanol (50 mL) followed by cold water. The resulting mixture was concentrated in vacuo. The residue diluted with water and extracted with dichloromethane (3×). The organic layer was dried over anhydrous Na₂SO₄, filtered and concentrated in vacuo. This material was used in the next step without any further purification.

### Step H

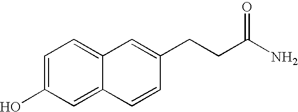

To a solution of the intermediate from step G (3.0 g, 12.3 mmol) in 1,4-dioxane (50 mL) placed in a pressure tube was added concentrated NH₄OH solution. The resulting mixture was stirred at room temperature for 18 hours. The reaction mixture was concentrated in vacuo and the residue was suspended in ethyl acetate, washed with water, dried over anhydrous sodium sulfate filtered and concentrated in vacuo. The residue was purified by flash chromatography using 50% ethyl acetate-hexanes then 100% ethyl acetate as the eluant to give the desired product as an off-white solid.

### Step I

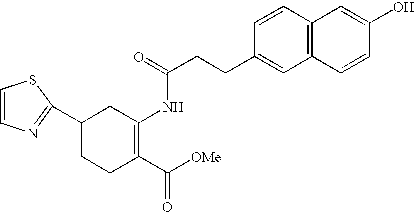

To a solution of the intermediate from step D (100 mg, 0.27 mmol) in anhydrous dioxane (2 mL) was added the intermediate from step H (48 mg, 0.22 mmol), XANTPHOS (31 mg, 0.053 mmol), cesium carbonate (122 mg, 0.376 mmol) and Pd₂(dba)₃ (15 mg, 0.016 mmol). The resulting mixture was de-gassed for 2 minutes by bubbling N₂. The reaction was heated at 50° C. under a N₂ atmosphere for two hours. The reaction mixture was cooled to room temperature, and filtered through celite. The filtrate was concentrated in vacuo and purified by flash chromatography using 35% ethyl acetate-hexanes to give the desired product.

### Step J

To a solution of the intermediate from step I (52 mg, 0.112 mmol) in THF (2 mL) was added 1N NaOH (1 mL) followed by MeOH (1 mL). The resulting mixture was stirred at room temperature for 5 hours. The reaction was quenched by the addition of 1N HCl (1 mL). The resulting mixture was concentrated in vacuo. The residue was extracted with ethyl acetate (3×). The combined organic layers were washed with brine, dried over anhydrous sodium sulfate, filtered and concentrated in vacuo. The residue was purified by reverse phase HPLC (Gilson) to give the desired product. ¹H NMR δ (500 MHz, DMSO) 11.62 (s, 1H), 7.75 (d, 1H), 7.68 (d, 1H), 7.55 (m, 2H), 7.3 (dd, 1H), 7.05 (m, 2H), 3.4 (dd, 1H), 3.3 (m, 1H), 3.15 (m, 1H), 2.95 (t, 2H), 2.66 (t, 2H), 2.35 (m, 2H), 2.07 (m, 1H), 1.72 (m, 1H). LCMS m/z 423 (M+1).

### Example 2

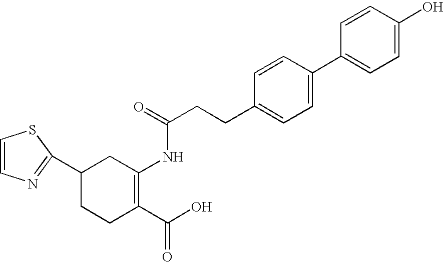

### Step A

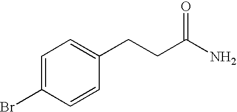

To a solution of 3-(4-bromophenyl) propionic acid (4.0 g, 17.46 mmol) in DCM (50 mL) was added N-hydroxy succinimide (4.02 g, 34.93 mmol) and EDC (6.7 g, 34.93 mmol). After stirring the reaction at room temperature for 18 hours, it was concentrated in vacuo. The resulting mixture was suspended in ethyl acetate and washed with water (2×). The organic layer was dried over anhydrous sodium sulfate, filtered and concentrated in vacuo. The residue was dissolved in 1,4-dioxane (100 mL). Concentrated ammonium hydroxide solution (100 mL) was added after stirring the reaction for 30 minutes it was concentrated in vacuo. The residue was suspended in ethyl acetate and washed with water (2×) and brine (1×). The organic layer was dried over anhydrous sodium sulfate, filtered and concentrated in vacuo to give the desired product as a white solid.

### Step B

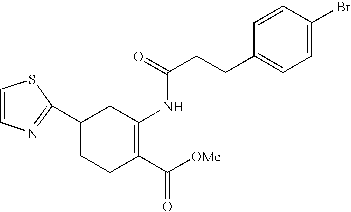

To a solution of the intermediate from example 1 step D (100 mg, 0.27 mmol) in anhydrous dioxane (2 mL) was added 3-(4-bromophenyl) propanamide the intermediate from step A (48 mg, 0.22 mmol), XANTPHOS (31 mg, 0.053 mmol), cesium carbonate (122 mg, 0.376 mmol) and Pd₂(dba)₃ (15 mg, 0.016 mmol). The resulting mixture was de-gassed for 2 minutes by bubbling N₂. The reaction was heated at 50° C. under a N₂ atmosphere for two hours. The reaction mixture was cooled to room temperature, and filtered through celite. The filtrate was concentrated in vacuo and purified by flash chromatography using 20% ethyl acetate-hexanes to give the desired product.

### Step C

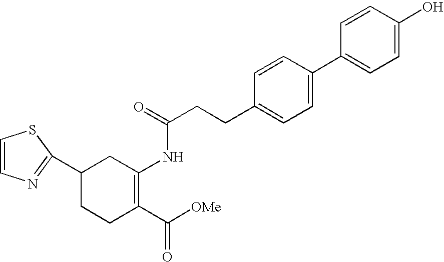

To a solution of the intermediate from step B (38 mg, 0.084 mmol) in THF (1.5 mL) was added 4-hydroxy phenyl boronic acid (18 mg, 0.127 mmol), K₂CO₃ (1 mL, 1.0 M solution) followed by 1,1′-bis(di-tert-butylphosphino)ferrocene palladium dichloride (10 mg). After heating the reaction at 50° C. for 1 hour, it was cooled to room temperature diluted with water and extracted with ethyl acetate (3×). The combined organic layers were washed with brine, dried over anhydrous sodium sulfate, filtered and concentrated in vacuo. The residue was purified by flash chromatography using 40% ethyl acetate-hexanes to give the desired product.

### Step D

To a solution of the intermediate from step C (27 mg) in THF (2 mL) was added 1N NaOH (1 mL) followed by MeOH (1 mL). The resulting mixture was stirred at room temperature for 5 hours. The reaction was quenched by the addition of 1N HCl (1 mL). The resulting mixture was concentrated in vacuo. The residue was extracted with ethyl acetate (3×). The combined organic layers were washed with brine, dried over anhydrous sodium sulfate, filtered and concentrated in vacuo. The residue was purified by reverse phase HPLC (Gilson) to give the desired product. ¹H NMR δ (500 MHz, DMSO) 11.62 (s, 1H), 7.75 (d, 1H), 7.63 (d, 1H), 7.48 (m, 3H), 7.26 (d, 2H), 6.82 (d, 2H), 3.4 (dd, 1H), 3.3 (m, 1H), 3.1 (m, 1H), 2.9 (t, 2H), 2.65 (t, 2H), 2.4 (m, 2H), 2.1 (m, 1H), 1.7 (m, 1H). LCMS m/z 448.15 (M+1).

### Example 3

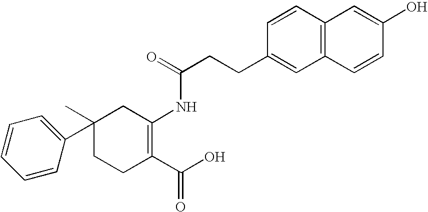

### Step A

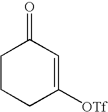

To a solution of cyclohexane 1,3-dione (1.0 g, 8.92 mmol) and 2,6-lutidine (2.07 mL, 17.84 mmol) in DCM cooled to 0° C. was added trifluoromethane sulfonic anhydride (2.25 mL, 13.38 mmol). The reaction mixture was stirred at room temperature for 30 minutes and quenched by the addition of 1N HCl. The resulting mixture was extracted with DCM. The organic layer was washed with 1N HCl, dried over anhydrous sodium sulfate, filtered and concentrated in vacuo. The residue was purified by flash chromatography using 20% ethyl acetate hexanes to give the desired product a light brown oil.

### Step B

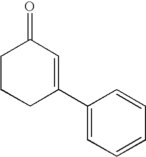

To a solution of the intermediate from step A (1.0 g, 4.09 mmol) in THF (5 mL) was added phenyl boronic acid (749 mg, 6.13 mmol), Na₂CO₃ (3 ml, 11.0M solution) and dichlorobis(triphenylphosphine)palladium (144 mg, 0.2 mmol). After heating the reaction mixture at 50° C. for 30 minutes it was cooled to room temperature and diluted with ethyl acetate. The organic layer was washed with brine, dried over anhydrous sodium sulfate, filtered and concentrated in vacuo. The residue was purified by flash chromatography using 10% ethyl acetate-hexanes to give the desired compound as a white solid.

### Step C

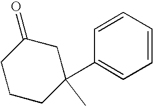

To a suspension of copper(I) iodide (3.77 g, 19.8 mmol) in anhydrous diethyl ether (30 mL) cooled to OC under a N₂ atmosphere was added drop-wise methyl lithium (24.8 mL, 39.6 mmol). After 15 minutes, the reaction mixture was cooled to −78° C. and a solution of the intermediate from step B (0.69 g, 3.96 mmol) in ether (20 mL) was added. The reaction was slowly warmed to room temperature and stirred for 1 hour. The reaction was quenched by the addition of saturated ammonium chloride solution. The resulting bi-phasic mixture was filtered through celite and washed extensively with ethyl acetate. The layers in the filtrate were separated and the aqueous layer extracted with ethyl acetate. The organic layer was washed with brine, dried over anhydrous sodium sulfate, filtered and concentrated in vacuo. The residue was purified by flash chromatography using 5% ethyl acetate hexanes to give the desired compound.

### Step D

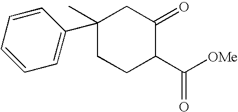

To a solution of the intermediate from step C (0.64 g, 3.36 mmol) in anhydrous THF (20 mL) cooled to −78° C. was added LHMDS (41 mL, 4.04 mmol, 1.0 M in THF). After 20 minutes, methyl cyanoformate (0.32 mL, 4.04 mmol) was added. The reaction was slowly warmed to −20° C. and quenched with 1N HCl. The resulting mixture was extracted with ethyl acetate (3×). The combined organic layers were washed with brine, dried over anhydrous sodium sulfate, filtered and concentrated in vacuo. The residue was purified by flash chromatography using 10% ethyl acetate-hexanes to give the desired product.

### Step E

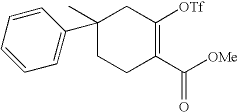

To a solution of the intermediate from step D (0.548 g, 2.22 mmol) in anhydrous THF (20 mL) cooled to 0° C. was added sodium hydride (0.133 g, 3.34 mmol, 60% by weight). After 30 minutes, 2-[N,N-Bis(trifluoromethylsulfonyl)amino]-5-chloropyridine (1.04 g, 2.66 mmol) was added. The reaction mixture was stirred at room temperature for two hours and then quenched with saturated ammonium chloride solution. The resulting mixture was extracted with ethyl acetate (3×). The combined organic layers were washed with brine, dried over anhydrous sodium sulfate, filtered and concentrated in vacuo. The residue was purified by flash chromatography using 2% then 5% ethyl acetate-hexanes to give the desired product as a colorless oil.

### Step F

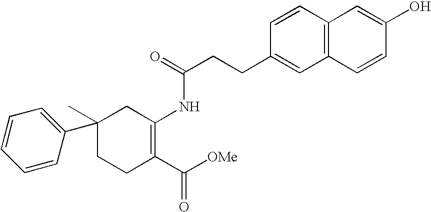

To a solution of the intermediate from step E (100 mg, 0.26 mmol) in anhydrous dioxane (2 mL) was added the intermediate from example 21 step H (48 mg, 0.22 mmol), XANTPHOS (30 mg, 0.052 mmol), cesium carbonate (120 mg, 0.369 mmol) and Pd₂(dba)₃ (15 mg, 0.016 mmol). The resulting mixture was de-gassed for 2 minutes by bubbling N₂ and heated at 50° C. under a N₂ atmosphere for two hours. The reaction mixture was cooled to room temperature, and filtered through celite. The filtrate was concentrated in vacuo and purified by flash chromatography using 20% ethyl acetate-hexanes to give the desired product.

### Step G

To a solution of the intermediate from step F (47 mg) in THF (4 mL) was added 1N NaOH (3 mL) followed by MeOH (1 mL). The resulting mixture was stirred at room temperature for 5 hours. The reaction was quenched by the addition of 1N HCl (3 mL). The resulting mixture was concentrated in vacuo. The residue was extracted with ethyl acetate (3×). The combined organic layers were washed with brine, dried over anhydrous sodium sulfate, filtered and concentrated in vacuo. The residue was purified by reverse phase HPLC (Gilson) to give the desired product. ¹H NMR δ (500 MHz, DMSO) 12.54 (s, 1H), 11.54 (s, 1H), 9.61 (s, 1H), 7.69 (d, 1H), 7.61 (d, 2H), 7.32 (d, 2H), 7.2 (m, 3H), 7.16 (m, 1H), 7.05 (m, 2H), 3.49 (d, 1H), 3.0 (t, 2H), 2.83 (d, 1H), 2.72 (t, 2H), 2.26 (m, 1H), 2.0 (m, 1H), 1.85 (m, 1H), 1.64 (m, 1H), 1.2 (s, 3H). LCMS m/z 430.2 (M+1).

### Example 4

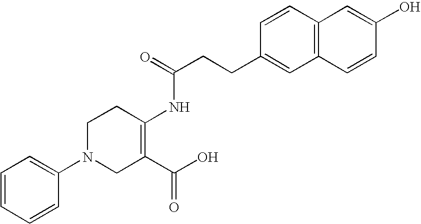

### Step A

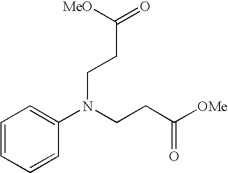

To a solution of aniline (2 mL, 21.69 mmol) in methanol (20 mL) placed in a pressure tube was added methyl acrylate (6.05 mL, 67.23 mmol), copper(I) chloride (400 mg, 4.03 mmol) and acetic acid (2.4 mL, 40.03 mmol). The resulting mixture was heated to reflux for 18 hours. The reaction was cooled to room temperature washed with water, 10% aqueous ammonia solution, water and brine. The organic layer was dried over anhydrous sodium sulfate, filtered and concentrated in vacuo. The residue was purified by flash chromatography using 20% ethyl acetate-hexanes to give the desired product as a colorless oil.

### Step B

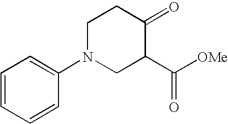

To a solution of the intermediate from step A (2.65 g, 10 mmol) in anhydrous dichloromethane (20 mL) cooled to −20° C. under a N₂ atmosphere was added titanium tetrachloride (10 mL, 10 mmol, 1.0M in THF). After stirring the reaction between −20° C. and −5° C. for two hours, triethyl amine (3.06 mL, 22 mmol) was added drop-wise over 10 minutes. The reaction was warmed to room temperature and left stirring for 18 hours. The reaction was quenched by pouring into a saturated solution of NaCl. Triethyl amine was added until the pH=8. The resulting mixture was filtered through celite. The filtrate was extracted with dichloromethane. The organic layer was dried over anhydrous sodium sulfate, filtered and concentrated in vacuo. The residue was purified by flash chromatography using 10% ethyl acetate-hexanes to give the desired compound as a yellow oil.

### Step C

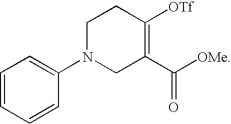

To a solution of the intermediate from step B (1.0 g, 4.29 mmol) in anhydrous THF (40 mL) cooled to 0° C. was added sodium hydride (0.258 g, 6.64 mmol, 60% by weight). After 30 minutes, 2-[N,N-Bis(trifluoromethylsulfonyl)amino]-5-chloropyridine (2.02 g, 5.14 mmol) was added. The reaction mixture was stirred at room temperature for 18 hours and then quenched with saturated ammonium chloride solution. The resulting mixture was extracted with ethyl acetate (3×). The combined organic layers were washed with brine, dried over anhydrous sodium sulfate, filtered and concentrated in vacuo. The residue was purified by flash chromatography using 15% ethyl acetate-hexanes to give the desired product as a yellow solid.

### Step D

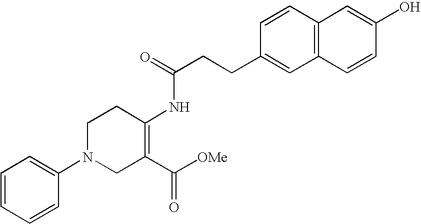

To a solution of the intermediate from step C (110 mg, 0.3 mmol) in anhydrous dioxane (2 mL) was added the intermediate from example 1 step H (54 mg, 0.25 mmol), XANTPHOS (16 mg, 0.027 mmol), cesium carbonate (137 mg, 0.421 mmol) and Pd₂(dba)₃ (8 mg, 0.009 mmol). The resulting mixture was de-gassed for 2 minutes by bubbling N₂. The reaction was heated at 50° C. under a N₂ atmosphere for 18 hours. The reaction mixture was cooed to room temperature, and filtered through celite. The filtrate was concentrated in vacuo and purified by flash chromatography using 20% ethyl acetate-hexanes to give the desired product.

### Step E

To a solution of the intermediate from step D (57 mg) in THF (2 mL) was added 1N NaOH (1 mL) followed by MeOH (1 mL). The resulting mixture was stirred at room temperature for 5 hours. The reaction was quenched by the addition of 1N HCl (1 mL). The resulting mixture was concentrated in vacuo. The residue was extracted with ethyl acetate (3×). The combined organic layers were washed with brine, dried over anhydrous sodium sulfate, filtered and concentrated in vacuo. The residue was purified by reverse phase HPLC (Gilson) to give the desired product. ¹H NMR δ (500 MHz, DMSO) 11.52 (s, 1H), 7.65 (d, 1H), 7.55 (m, 2H), 7.3 (d, 1H), 7.25 (m, 2H), 7.1 (m, 2H), 6.9 (d, 2H), 6.8 (t, 1H), 3.85 (bs, 1H), 3.48 (t, 1H), 3.36 (t, 2H), 3.05 (bt, 2H), 2.95 (t, 2H), 2.7 (t, 2H). LCMS m/z 417.2 (M+1).

### Example 5

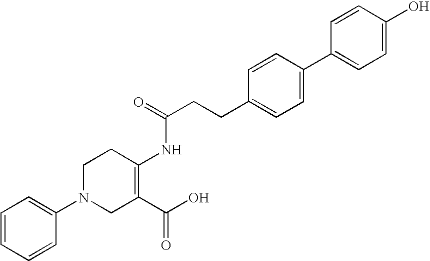

### Step A

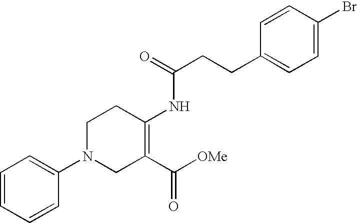

To a solution of the intermediate from example 4 step C (220 mg, 0.6 mmol) in anhydrous dioxane (4 mL) was added the intermediate from example 22 step A (114 mg, 0-5 mmol), XANTPHOS (32 mg, 0.05 mmol), cesium carbonate (275 mg, 0.846 mmol) and Pd₂(dba)₃ (16 mg, 0.017 mmol). The resulting mixture was de-gassed for 2 minutes by bubbling N₂. The reaction was heated at 50° C. under a N₂ atmosphere for 18 hours. The reaction mixture was cooled to room temperature, and filtered through celite. The filtrate was concentrated in vacuo and purified by flash chromatography using 5% ethyl acetate-hexanes to give the desired product.

### Step B

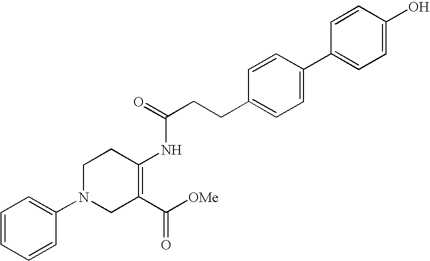

To a solution of the intermediate from step A (50 mg, 0.112 mmol) in THF (1 mL) was added 4-hydroxy phenyl boronic acid (23 mg, 0.168 mmol), Na₂CO₃ (1 mL, 10.0 M solution) followed by and dichlorobis(triphenylphosphine)palladium (4 mg, 0.005 mmol). After heating the reaction mixture at 50° C. for 30 minutes, it was cooled to room temperature and diluted with ethyl acetate. The organic layer was washed with brine, dried over anhydrous sodium sulfate, filtered and concentrated in vacuo. The residue was purified by flash chromatography using 20% ethyl acetate-hexanes to give the desired compound as a white solid.

### Step C

To a solution of the intermediate from step B (57 mg) in THF (2 mL) was added 1N NaOH (1 mL) followed by MeOH (1 mL). The resulting mixture was stirred at room temperature for 5 hours. The reaction was quenched with 1N HCl (1 mL). The resulting mixture was concentrated in vacuo. The residue was extracted with ethyl acetate (3×). The combined organic layers were washed with brine, dried over anhydrous sodium sulfate, filtered and concentrated in vacuo. The residue was purified by reverse phase HPLC (Gilson) to give the desired product. ¹H NMR δ (500 MHz, DMSO) 11.52 (s, 1H), 7.45 (m, 4H), 7.25 (m, 4H), 6.96 (d, 2H), 6.8 (m, 3H), 3.86 (bs, 1H), 3.45 (bt, 1H), 3.37 (t, 2H), 3.05 (bt, 2H), 2.87 (t, 2H), 2.67 (t, 2H). LCMS m/z 443.2 (M+1).

### Example 6

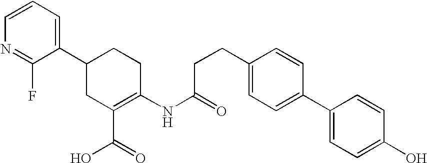

### Step A

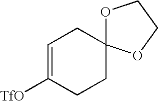

To a solution of 1,4-cyclohexane dione mono-ethylene ketal (4.0 g, 25.61 mmol) in anhydrous THF (130 mL) cooled to −78° C. under a N₂ atmosphere was added LDS (28 mL, 28 mmol, 1.0 M in THF). After stirring for 1 hour a solution 2-[N,N-Bis(trifluoromethylsulfonyl)amino]-5-chloropyridine (10.0 g, 25.46 mmol) in THF (100 mL) was added. The reaction was warmed to room temperature and stirred for 18 hours. The reaction was quenched with water and the resulting mixture was extracted with ethyl acetate (3×). The combined organic layers were washed with brine, dried over anhydrous sodium sulfate, filtered and concentrated in vacuo. The residue was purified by flash chromatography (Biotage, Horizon) using (0% EtOAc/Hexane→20% EtOAc/Hexane) to give the desired product as a colorless oil.

### Step B

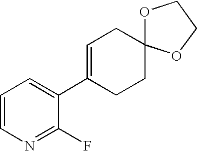

To a solution of the intermediate from step A (7.00 g, 24.29 mmol) in THF (200 mL) was added 2-fluoro-3-pyridine boronic acid (3.42 g, 24.29 mmol), and tetrakis triphenyl phosphine palladium (0) (1.00 g, 0.900 mmol). Aqueous sodium carbonate solution (1M, 48 mL) was added, the reaction mixture was flushed with N₂ and heated to 50° C. for 1 hour. The reaction was cooled to room temperature, diluted with ethyl acetate, washed with brine, and dried over sodium sulfate. The crude material was purified by flash chromatography (Bioatage Horizon) (20% EtOAc/Hexane→40% EtOAc/Hexane) to give the desired product.

### Step C

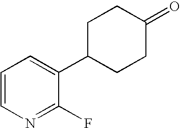

To a solution of the intermediate from step B (5.71 g, 24.3 mmol) in MeOH (10 mL) was added palladium on carbon (5%, 2 g) in MeOH (10 mL). The reaction mixture was stirred under a hydrogen balloon for 18 hours, and then filtered through celite and concentrated in vacuo. The crude material was dissolved in THF/EtOH (100 mL/40 mL) and HCl (80 mL, 3N) was added. The resulting mixture was stirred at room temperature for 18 hours. The reaction mixture was concentrated in vacuo. The residue was diluted with ethyl acetate, and adjusted to pH=8 with 1 N NaOH. The resulting mixture was extracted with EtOAc (2×), washed with brine and dried over Na₂SO₄, filtered and concentrated in vacuo. The crude material was purified by flash chromatography (Biotage Horizon) (0% EtOAc/Hexane→60% EtOAc/Hexane) to give the desired product.

### Step D

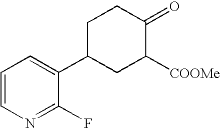

To a solution of the intermediate from step C (1.18 g, 6.11 mmol) in anhydrous THF (61 mL) cooled to −78° C. under a N₂ atmosphere was added LHMDS (6.16 mL, 9.16 mmol, 1.0 M in THF). After 1 hour, methyl cyanoformate (0.686 mL, 8.54 mmol) was added and the reaction was allowed to warm to 40° C. over 2 hours. The reaction was quenched with 1N HCl and extracted with EtOAc (2×). The organic layer was washed with brine and dried over Na₂SO₄, filtered and concentrated in vacuo. This material was used in the next step without any further purification.

### Step E

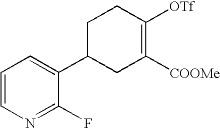

To a solution of the intermediate from step D (1.54 g, 6.11 mmol) in anhydrous THF cooled to 0° C. was added NaH (366 mg, 9.16 mmol, 60%). After 30 minutes, a solution of 2-[N,N-Bis(trifluoromethylsulfonyl)amino]-5-chloropyridine (2.88 g, 7.33 mmol) in THF (20 mL) was added. The reaction was stirred at room temperature for 18 hours and then quenched with water. The resulting mixture was extracted with EtOAc (2×). The organic layer was washed with brine, dried over Na₂SO₄, filtered, and concentrated in vacuo. The crude material was purified by flash chromatography (Biotage, Horizon) (0% EtOAc/Hexane+30% EtOAc/Hexane) to give the desired product.

### Step F

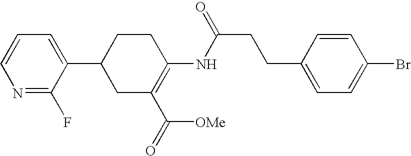

To a solution of the intermediate from E (700 mg, 1.83 mmol) in anhydrous dioxane (18 mL) was added the intermediate from example 2 step A (416 mg, 1.83 mmol), XANTPHOS (95 mg, 0.165 mmol), cesium carbonate (832 mg, 2.56 mmol) and Pd₂(dba)₃ (50 mg, 0.055 mmol). The resulting mixture was de-gassed for 2 minutes by bubbling N₂. The reaction was heated at 55° C. under a N₂ atmosphere for 18 hours. The reaction mixture was cooled to room temperature, and filtered through celite. The filtrate was concentrated in vacuo and purified by flash chromatography (Biotage, Horizon) using 30% ethyl acetate-hexanes to give the desired product. This intermediate was resolved into its enantiomers using a Chiral IA HPLC column, isocratic elution with 15% ethanol-heptanes, and a 45 minute elution time.

### Step G

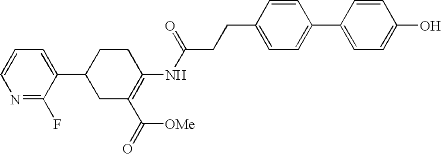

To a solution of the intermediate from step F (300 mg, 0.65 mmol) in THF (5 mL) was added 4-hydroxy phenyl boronic acid (134 mg, 0.975 mmol), K₂CO₃ (5 mL, 1.0 M solution) followed by (1,2′-bis(di-t-butylphosphino)ferrocene palladium dichloride, 13 mg, 0.02 mmol). After heating the reaction mixture at 100° C. for 2 hours it was cooled to room temperature and diluted with ethyl acetate. The organic layer was washed with brine, dried over anhydrous sodium sulfate, filtered and concentrated in vacuo. The residue was purified by flash chromatography (Biotage, Horizon) (0% EtOAc/Hexane→100% EtOAc/Hexane) to give the desired compound.

### Step H

To a solution of the intermediate from step G (230 mg) in THF (4.5 mL) was added 1N LiOH (3 mL) followed by MeOH (1.5 mL). The resulting mixture was stirred at room temperature for 18 hours. The reaction was neutralized with 1N HCl (3 mL). The resulting mixture was concentrated in vacuo. The residue was extracted with ethyl acetate (3×). The combined organic layers were washed with brine, dried over anhydrous sodium sulfate, filtered and concentrated in vacuo. The residue was purified by reverse phase HPLC (Gilson) to give the desired product, including the resolved enantiomers. ¹H NMR δ (500 MHz, CD₃OD) 8.04 (d, 1H), 7.82 (t, 1H), 7.46-7.40 (m, 4H), 7.28-7.24 (m, 3H), 6.84-6.81 (m, 2H), 3.20-2.96 (m, 5H), 2.73 (m, 1H), 2.65 (t, 2H), 2.34 (m, 1H), 1.97 (m, 1H), 1.82 (m, 1H) LCMS m/z 459 (M−1).

### Example 7

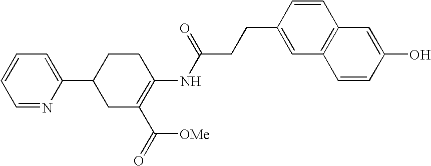

### Step A

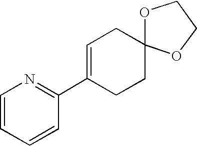

To a solution of the intermediate from example 6 step A (0.993 g, 3.35 mmol) in anhydrous THF (20 mL) was added 2-pyridyl-tri-n-butyl stannane (1.0 g, 4.13 mmol), copper (1) iodide (131 mg, 0.69 mmol) and dichlorobis(triphenylphosphine)palladium (242 mg, 0.345 mmol). The reaction was heated to reflux for 45 minutes under a N₂ atmosphere. The reaction was cooled to room temperature and saturated KF solution was added (20 mL). After stirring the resulting mixture for 45 minutes it was filtered through celite and concentrated in vacuo. The crude material was purified by flash chromatography (Biotage, Horizon) (hexane+30% EtOAc/lexane) yielding a pure orange oil.

### Step B

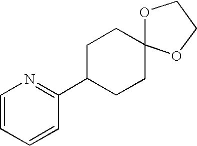

To a solution of the intermediate from step A (423 mg, 1.95 mmol) in MeOH (10 mL) was added Pd/C (400 mg) and the resulting mixture stirred under a hydrogen balloon for 4 hours. The reaction was filtered through celite and concentrated in vacuo. This material was used in the next step without any further purification.

### Step C

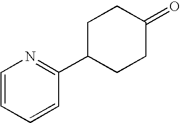

To a solution of the intermediate from step B (351 mg, 1.6 mmol) in 2:1 THF/EtOH (7.5 mL) was added HCl (5 mL, 3N). After stirring at room temperature for 18 hours the reaction mixture was concentrated in vacuo. The residue was neutralized with saturated sodium bicarbonate solution and extracted with ethyl acetate (2×). The organic layer was washed with brine, dried over anhydrous sodium sulfate filtered and concentrated in vacuo to give a brown oil. This material was used in the next step without any further purification.

### Step D

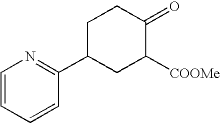

To a solution of the intermediate from step C (210 mg, 1.2 mmol) in anhydrous THF (8 mL) cooled to −78° C. was added LHMDS (1.32 mL, 1.32 mmol, 1.0 M in THF). The reaction was warmed to 0° C. and stirred for 30 minutes. The reaction was then cooled to −78° C. and methyl cyanoformate (0.114 mL, 1.44 mmol) was added. The reaction was warmed to −20° C. over 2 hours and quenched with 1N HCl. The resulting mixture was extracted with ethyl acetate (2×). The organic layer was washed with brine, dried over anhydrous sodium sulfate, filtered and concentrated in vacuo to give an orange oil. This material was used in the next step without any further purification.

### Step E

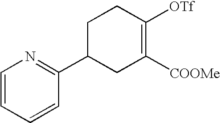

To a solution of the intermediate from step D (284 mg, 1.22 mmol) in anhydrous THF (9 mL) cooled to 0° C. was added NaH (58 mg, 1.46 mmol, 60%). After 30 minutes 2-[N,N-Bis(trifluoromethylsulfonyl)amino]-5-chloropyridine (485 mg, 1.22 mmol) in THF (2 mL) was added and the resulting reaction stirred at room temperature for 2 hours. The reaction was quenched with 1N HCl, then extracted with EtOAc (2×). The organic layers were washed with brine, dried over Na₂SO₄, filtered and concentrated in vacuo to give a brown oil. This material was purified by Prep-TLC (SiO₂) using 50% EtOAc/hexanes as eluant to give the desired product.

### Step F

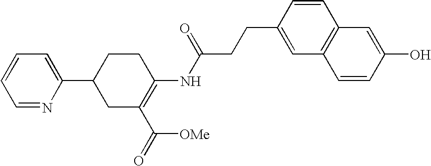

To a solution of the intermediate from step E (67 mg, 0.183 mmol) in anhydrous dioxane (2 mL) was added the intermediate from example 21 step H (39 mg, 0.183 mmol), XANTPHOS (10 mg, 0.016 mmol), cesium carbonate (83 mg, 0.256 mmol) and Pd₂(dba)₃ (6 mg, 0.006 mmol). The resulting mixture was de-gassed for 2 minutes by bubbling N₂. The reaction was heated at 50° C. under a N₂ atmosphere for 18 hours. The reaction mixture was cooled to room temperature, and filtered through celite. The filtrate was concentrated in vacuo and purified by Prep TLC (SiO₂) using 60% ethyl acetate-hexanes as eluant to give the desired product.

### Step G

To a solution of the intermediate from step F (37 mg, 0.085 mmol) in THF (2 mL) was added 1N NaOH (1 mL) followed by MeOH (1 mL). The resulting mixture was stirred at room temperature for 18 hours. The reaction was quenched by the addition of 1N HCl (1 mL). The resulting mixture was concentrated in vacuo. The residue was extracted with ethyl acetate (3×). The combined organic layers were washed with brine, dried over anhydrous sodium sulfate, filtered and concentrated in vacuo. The residue was purified by reverse phase HPLC (Gilson) to give the desired product. ¹H NMR δ (500 MHz, CD₃OD) 8.66 (d, 1H), 8.38 (t, 1H), 7.86 (d, 1H), 7.79 (t, 1H), 7.63 (d, 1H), 7.56 (d, 1H), 7.55 (s, 1H), 7.27 (m, 1H), 7.06-7.02 (m, 2H), 3.25-2.96 (m, 5H), 2.86 (d, 1H), 2.72 (t, 2H), 2.49 (m, 1H), 2.11 (m, 1H), 1.88 (m, 1H). LCMS m/z 417 (M+1).

### EXAMPLES 8-37

The following Examples were prepared under conditions similar to those described in Examples 1-7 above and illustrated in Schemes 1-8.

| EXAMPLE | | LCMS |
|---|---|---|
| 8 | 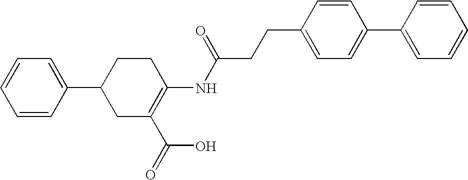 | (M − 1) = 424 |
| 9 | 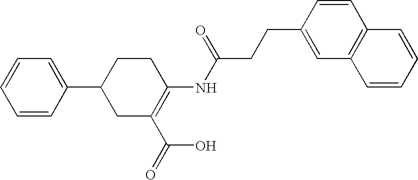 | (M − 1) = 398 |
| 10 | 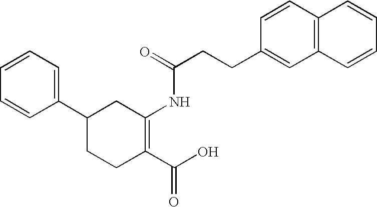 | (M + 1) = 400.2 |
| 11 | 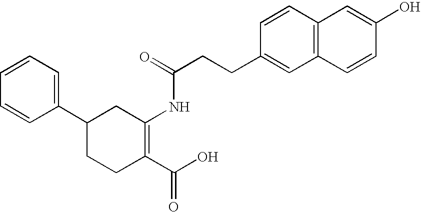 | (M + 1) = 416.2 |
| 12 | 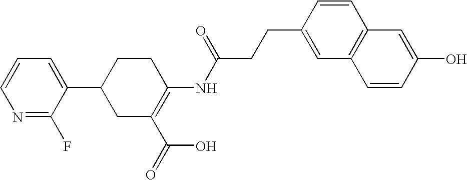 | (M − 1) = 433 |
| 13 | 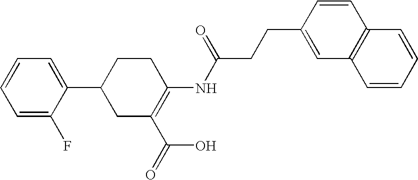 | (M − 1) = 416 |
| 14 | 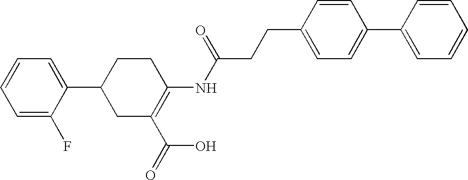 | (M − 1) = 442 |
| 15 | 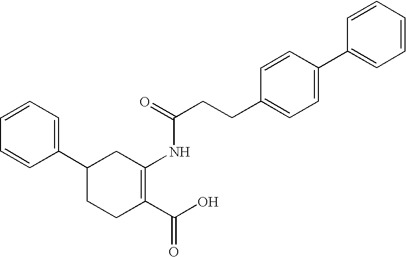 | (M + 1) = 426.2 |
| 16 | 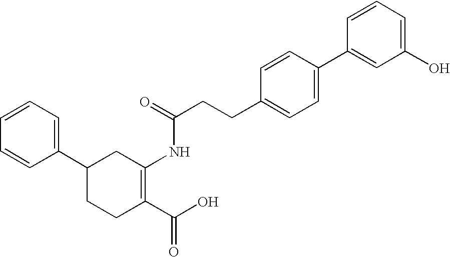 | (M + 1) = 442.2 |
| 17 | 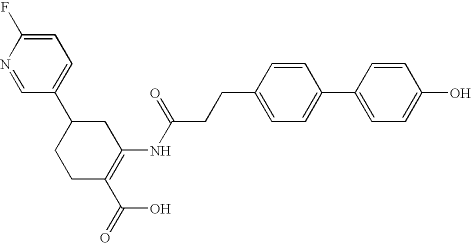 | (M + 1) = 461 |
| 18 | 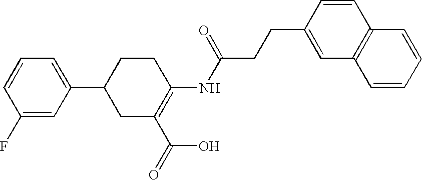 | (M + 1) = 418 |
| 19 | 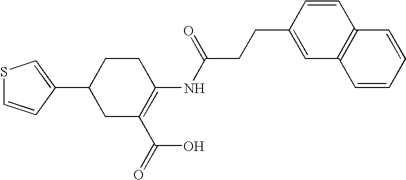 | (M − 1) = 404 |
| 20 | 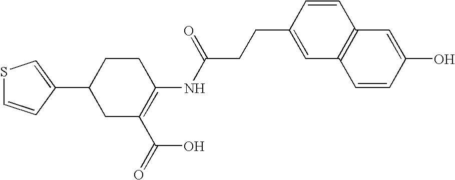 | (M − 1) = 420 |
| 21 | 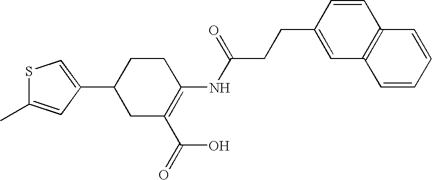 | (M − 1) = 418 |
| 22 | 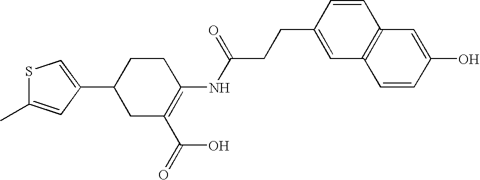 | (M − 1) = 434 |
| 23 | 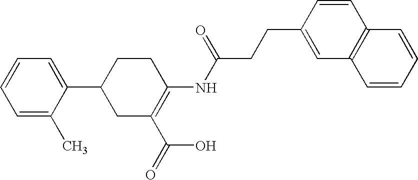 | (M − 1) = 412 |
| 24 | 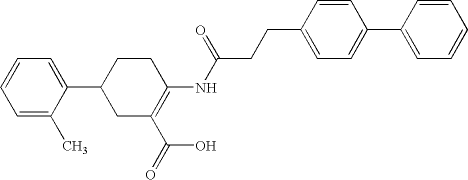 | (M − 1) = 438 |
| 25 | 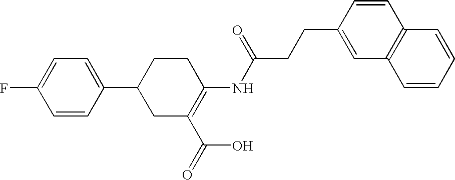 | (M − 1) = 416 |
| 26 | 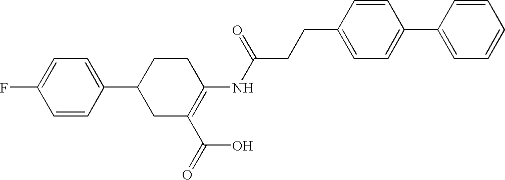 | (M − 1) = 442 |
| 27 | 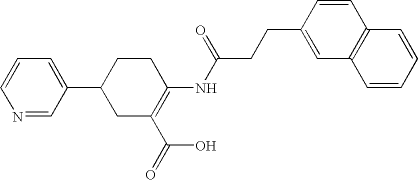 | (M − 1) = 399 |
| 28 | 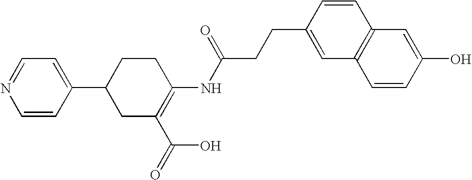 | (M + 1) = 417 |
| 29 | 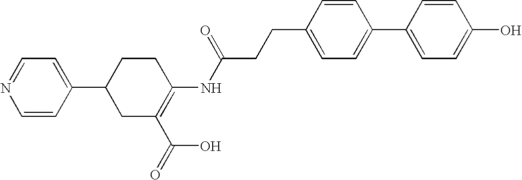 | (M − 1) = 441 |
| 30 | 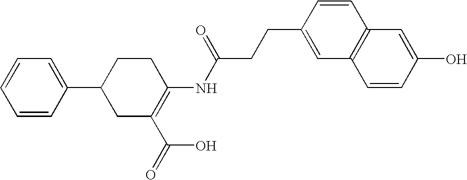 | (M − 1) = 414 |
| 31 | 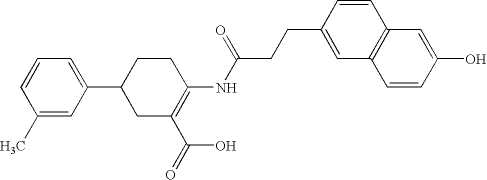 | (M − 1) = 428 |
| 32 | 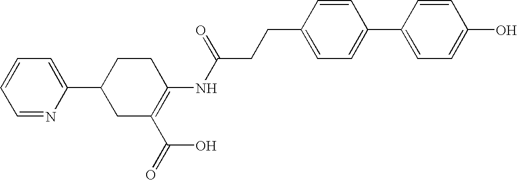 | (M − 1) = 441 |
| 33 | 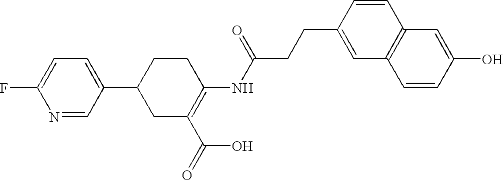 | (M − 1) = 433 |
| 34 | 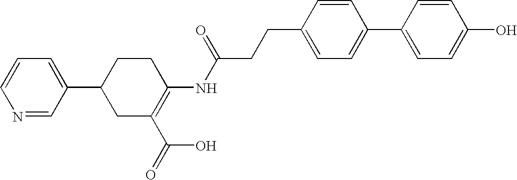 | (M − 1) = 441 |
| 35 | 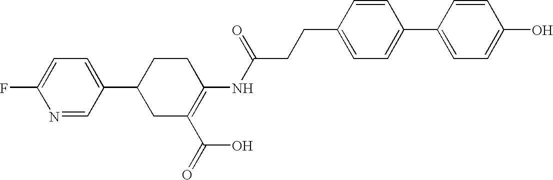 | (M + 1) = 461 |
| 36 | 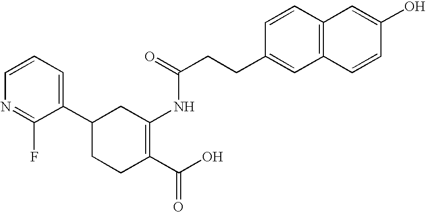 | (M + 1) = 435.3 |
| 37 | 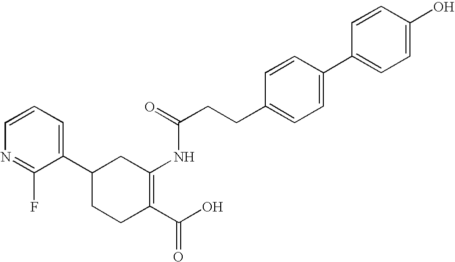 | (M + 1) = 461.2 |

NMR data for selected examples:

### Example 8

¹H NMR (500 MHz, DMSO) δ 7.58 (d, 2H), 7.53 (d, 2H), 7.41 (t, 2H), 7.32-7.16 (m, 8H), 3.16 (d, 1H), 3.02-2.91 (m, 3H), 2.76-2.65 (m, 3H), 2.31 (m, 1H), 1.96 (m, 1H), 1.75 (m, 1H), 1.28 (m, 1H)

### Example 9

¹H NMR (500 MHz, DMSO) δ 7.86-7.82 (m, 3H), 7.72 (s, 1H), 7.48-7.41 (m, 3H), 7.30-7.18 (m, 5H), 3.11-3.02 (m, 3H), 2.86 (m, 1H), 2.72-2.65 (m, 3H), 2.62-2.52 (m, 1H) 2.19 (m, 1H), 1.86 (m, 1H), 1.67 (m, 1H).

### Example 10

¹H NMR δ (500 MHz, DMSO) 7.79-7.75 (m, 3H), 7.65 (s, 1H), 7.44-7.35 (m, 3H), 7.3-7.2 (m, 2H), 7.2-7.16 (m, 3H), 3.2 (dd, 1H), 3.1 (t, 2H), 2.85 (m, 1H), 2.75 (m, 1H), 2.7 (t, 2H), 2.55 (m, 1H), 2.35 (m, 1H), 1.9 (m, 1H), 1.65 (m, 1H).

### Example 11

¹H NMR δ (500 MHz, DMSO) 12.62 (bs, 1H), 11.61 (s, 1H), 9.59 (s, 1H), 7.64 (d, 1H), 7.54 (d, 1H), 7.31 (s, 1H), 7.2 (m, 6H), 7.0 (m, 2H), 3.1 (d, 1H), 2.95 (t, 2H), 2.75 (m, 2H), 2.6 (t, 2H), 2.4 (m, 1H), 2.3 (m, 1H), 1.8 (m, 1H), 1.7 (m, 1H).

### Example 12

¹H NMR δ (500 MHz, CD₃OD) 8.04 (d, 1H), 7.82 (t, 1H), 7.64-7.56 (dd, 2H), 7.56 (s, 1H), 7.28-7.25 (m, 2H), 7.06-7.02 (m, 2H), 3.18-3.05 (m, 3H), 3.03-2.91 (m, 2H), 2.72-2.68 (m, 3H), 2.32 (m, 1H), 1.95 (m, 1H), 1.80 (m, 1H).

### Example 13

¹H NMR δ (500 MHz, DMSO) 7.86-7.82 (m, 3H), 7.72 (s, 1H), 7.48-7.41 (m, 3H), 7.32-7.23 (m, 2H), 7.16-7.11 (m, 2H), 3.12-2.83 (m, 5H), 2.71 (t, 2H), 2.92-2.48 (m, 1H), 2.21 (m, 1H), 1.83 (m, 1H), 1.75 (m, 1H).

### Example 14

¹H NMR δ (500 MHz, DMSO) 7.62 (d, 2H), 7.58 (d, 2H), 7.44 (t, 2H), 7.34-7.23 (m, 5H), 7.16-7.12 (m, 2H), 3.12 (d, 1H), 2.99 (m, 1H), 2.92-2.84 (m, 3H), 2.65-2.55 (m, 3H), 2.22 (m, 1H), 1.85 (m, 1H), 1.77 (m, 1H).

### Example 15

¹H NMR δ (500 MHz, DMSO) 12.64 (bs, 1H), 11.62 (s, 1H), 7.67 (d, 2H), 7.56 (d, 2H), 7.4 (t, 2H), 7.3-7.2 (m, 8H), 3.2 (d, 1H), 2.85 (t, 2H), 2.75 (m, 2H), 2.6 (t, 2H), 2.42 (m, 1H), 2.26 (t, 1H), 1.82 (m, 1H), 1.65 (m, 1H).

### Example 16

¹H NMR δ (500 MHz, DMSO) 12.64 (bs, 1H), 11.62 (s, 1H), 9.46 (s, 1H), 7.48 (d, 2H), 7.31-7.19 (m, 8H), 6.96 (m, 2H), 6.71 (dd, 1H), 3.2 (d, 1H), 2.86 (t, 2H), 2.73 (m, 2H), 2.6 (t, 2H), 2.42 (m, 1H), 2.3 (m, 1H), 1.8 (m, 1H), 1.66 (m, 1H).

### Example 17

¹H NMR δ (500 MHz, DMSO) 11.6 (s, 1H), 9.48 (s, 1H), 8.12 (d, 1H), 7.91 (t, 1H), 7.44 (m, 4H), 7.23 (d, 2H), 7.11 (dd, 1H), 6.8 (dd, 2H), 3.2 (m, 1H), 2.9 (m, 3H), 2.6 (m, 3H), 2.45 (m, 1H), 2.2 (m, 1H), 1.8 (m, 2H).

### Example 18

1H NMR δ (500 MHz, DMSO) 7.86-7.82 (m, 3H), 7.72 (s, 1H), 7.48-7.31 (m, 4H), 7.34-7.30 (m, 2H), 7.09-7.08 (m, 1H), 3.11-3.02 (m, 3H), 2.85 (m, 1H), 2.76-2.52 (m, 3H) 2.19 (m, 1H), 1.86 (m, 1H), 1.67 (m, 1H), 1.22 (m, 1H).

### Example 19

¹H NMR δ (500 MHz, CD₃OD) 7.80-7.77 (m, 3H), 7.67 (s, 1H), 7.43-7.30 (m, 4H), 7.05-7.01 (m, 2H), 3.14-3.07 (m, 3H), 2.95-2.81 (m, 2H), 2.75-2.70 (m, 3H), 2.30 (m, 1H), 2.03 (m, 1H), 1.66 (m, 1H).

### Example 20

¹H NMR δ (500 MHz, CD₃OD) 7.64-7.55 (dd, 2H), 7.55 (s, 1H), 7.32-7.25 (m, 2H), 7.06-7.01 (m, 4H), 3.11-3.04 (m, 3H), 2.95-2.81 (m, 2H), 2.75-2.66 (m, 3H), 2.30 (m, 1H), 2.03 (m, 1H), 1.66 (m, 1H).

### Example 21

¹H NMR δ (500 MHz, CD₃OD) 7.80-7.77 (m, 3H), 7.68 (s, 1H), 7.44-7.37 (m, 3H), 6.97-6.96 (m, 2H), 3.16-3.08 (m, 3H), 2.98 (m, 1H), 2.80-2.67 (m, 4H), 2.24 (m, 1H), 2.20 (s, 3H), 1.96 (m, 1H), 1.62 (m, 1H).

### Example 22

¹H NMR δ (500 MHz, CD₃OD) 7.64-7.55 (dd, 2H), 7.55 (s, 1H), 7.28-7.26 (dd, 1H), 7.06-6.95 (m, 4H), 3.16-3.05 (m, 3H), 2-94 (m, 1H), 2.78-2.67 (m, 4H), 2.24 (m, 1H), 2.20 (s, 3H), 1.96 (m, 1H), 1.64 (m, 1H).

### Example 23

¹H NMR δ (500 MHz, DMSO) 7.77-7.74 (m, 3H), 7.65 (s, 1H), 7.40-7.34 (m, 3H), 7.14-6.99 (m, 4H), 3.05-2.99 (m, 3H), 2.91-2.81 (m, 2H), 2.68-2.61 (m, 3H) 2.23 (s, 3H), 2.10 (m, 1H), 1.72 (m, 1H), 1.65 (m, 1H).

### Example 24

¹H NMR δ (500 MHz, DMSO) 7.62 (d, 2H), 7.57 (d, 2H), 7.43 (t, 2H), 7.34-7.31 (m, 3H), 7.18-7.05 (m, 4H), 3.13-3.09 (m, 1H), 2.93-2.87 (m, 3H), 2.65-2.61 (m, 2H), 2.54-2.94 (m, 2H) 2.27 (s, 3H), 2.13 (m, 1H), 1.78 (m, 1H), 1.70 (m, 1H).

### Example 25

¹H NMR δ (500 MHz, DMSO) 7.86-7.81 (m, 3H), 7.72 (s, 1H), 7.48-7.40 (m, 3H), 7.28-7.25 (m, 2H), 7.09 (t, 2H), 3.09-3.02 (m, 3H), 2.85 (m, 1H), 2.73-2.68 (m, 3H), 2.54 (m, 1H), 2.17 (m, 1H), 1.84 (m, 1H), 1.66 (m, 1H).

### Example 26

¹H NMR δ (500 MHz, DMSO) 7.62 (d, 2H), 7.57 (d, 2H), 7.44 (t, 2H), 7.34-7.26 (m, 5H), 7.10 (t, 2H), 3.09 (m, 1H), 2.92-2.80 (m, 3H), 2.71 (m, 1H), 2.65-2.62 (m, 2H), 2.55 (m, 1H), 2.19 (m, 1H), 1.85 (m, 1H), 1.67 (m, 1H).

### Example 27

¹H NMR δ (500 MHz, CD₃OD) 8.75 (s, 1H), 8.68 (d, 1H), 8.49 (d, 1H), 7.95 (m, 1H), 7.81-7.77 (m, 3H), 7.68 (s, 1H), 7.45-7.37 (m, 3H), 3.23-3.12 (m, 3H), 3.07-2.98 (m, 2H), 2.83-2.74 (m, 3H), 2.40 (m, 1H), 2.06 (m, 1H), 1.85 (m, 1H).

### Example 28

¹H NMR δ (500 MHz, CD₃OD) 8.69 (d, 2H), 7.89 (d, 2H), 7.63 (d, 1H), 7.56 (d, 1H), 7.55 (s, 1H), 7.27 (d, 1H), 7.06-7.02 (m, 2H), 3.21-2.97 (m, 5H), 2.81-2.69 (m, 3H), 2.40 (m, 1H), 2.06 (m, 1H), 1.83 (m, 1H).

### Example 29

¹H NMR δ (500 MHz, CD₃OD) 8.71 (d, 2H), 7.95 (d, 2H), 7.46-7.24 (m, 6H), 6.84-6.81 (m, 2H), 3.23-2.97 (m, 5H), 2.82 (m, 2H), 2.66 (t, 1H), 2.43 (m, 1H), 2.08 (m, 1H), 1.87 (m, 1H).

### Example 30

¹H NMR δ (500 MHz, DMSO) 7.65 (d, 1H), 7.58 (d, 1H), 7.56 (s, 1H), 7.30-7.16 (m, 7H), 7.05-7.01 (m, 2H), 3.09 (d, 1H), 2.96 (t, 2H), 2.86 (m, 1H), 2.70-2.52 (m, 3H), 2.20 (m, 1H), 1.86 (m, 1H), 1.66 (m, 1H), 1.22 (m, 1H).

### Example 31

¹H NMR δ (500 MHz, CD₃OD) 7.65 (d, 1H), 7.58 (d, 1H), 7.56 (s, 1H), 7.27 (d, 1H), 7.12-7.01 (m, 6H) 3.06 (d, 1H), 2.96 (t, 1H), 2.85 (m, 1H), 2.67-2.62 (m, 3H), 2.54-2.48 (m, 1H), 2.24 (s, 3H), 2.17 (m, 1H), 1.83 (m, 1H), 1.64 (m, 1H), 1.22 (m, 1H).

### Example 32

¹H NMR δ (500 MHz, CD₃OD) 8.66 (d, 1H), 8.38 (t, 1H), 7.86 (d, 1H), 7.78 (t, 1H), 7.46-7.41 (m, 4H), 7.25 (d, 2H), 6.84-6.82 (m, 2H), 3.23-2.97 (m, 5H), 2.87 (d, 1H), 2.67 (t, 2H), 2.52 (m, 1H), 2.14 (m, 1H), 1.91 (m, 1H).

### Example 33

¹H NMR δ (500 MHz, CD₃OD) 8.07 (s, 1H), 7.86 (m, 1H), 7.63 (d, 1H), 7.56 (d, 1H), 7.55 (s, 1H), 7.27 (d, 1H) 7.06-6.99 (m, 3H), 3.16 (m, 1H), 3.07 (t, 2H), 2.95 (m, 1H), 2.81 (m, 1H), 2.71-2.67 (m, 3H), 2.29 (m, 1H), 1.95 (m, 1H), 1.74 (m, 1H).

### Example 34

¹H NMR δ (500 MHz, CD₃OD) 8.75 (s, 1H), 7.67 (d, 1H), 7.46 (d, 1H), 7.94 (m, 1H), 7.46-7.40 (m, 4H), 7.25 (d, 2H), 6.84-6.81 (m, 2H), 3.22 (m, 1H), 3.08-2.97 (m, 4H), 2.82 (m, 1H), 2.66 (t, 2H), 2.41 (m, 1H), 2.07 (m, 1H), 1.85 (m, 1H)

### Example 35

¹H NMR δ (500 MHz, CD₃OD) 8.08 (s, 1H), 7.85 (t, 1H), 7.46-7.41 (m, 4H), 7.26 (d, 2H), 7.01 (dd, 1H), 6.84-6.82 (m, 2H), 3.18 (m, 1H), 2.99-2.96 (m, 3H), 2.84 (m, 1H), 2.72 (m, 1H), 2.65 (t, 2H), 2.32 (m, 1H), 1.97 (m, 1H), 1.77 (m, 1H).

### Example 36

¹H NMR δ (500 MHz, DMSO) 12.7 (bs, 1H), 11.62 (s, 1H), 9.61 (bs, 1H), 8.12 (d, 1H), 7.86 (t, 1H), 7.67 (d, 1H), 7.58 (m, 2H), 7.36 (t, 1H), 7.27 (d, 1H), 7.05 (m, 2H), 3.2 (dd, 1H), 3.0 (m, 1H), 2.94 (t, 2H), 2.85 (m, 1H), 2.65 (t, 2H), 2.42 (m, 1H), 2.32 (m, 1H), 1.83 (m, 1H), 1.73 (m, 1H).

### Example 37

¹H NMR δ (500 MHz, DMSO) 12.7 (bs, 1H), 11.61 (s, 1H), 9.5 (s, 1H), 8.12 (d, 1H), 7.88 (t, 1H), 7.45 (m, 4H), 7.34 (t, 1H), 7.25 (d, 2H), 6.82 (d, 2H), 3.25 (dd, 1H), 3.0 (m, 1H), 2.9 (t, 2H), 2.8 (m, 1H), 2.65 (t, 2H), 2.45 (m, 1H), 2.35 (m, 1H), 1.86 (m, 1H), 1.75 (m, 1H).

### Example 38

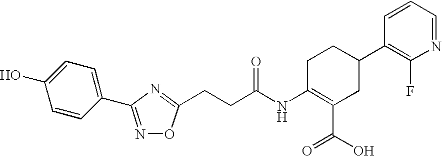

### Step A

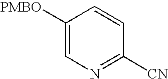

To a solution of 4-methoxy benzyl alcohol (3.77 mL, 30.4 mmol) in anhydrous DMF (35 mL) was added sodium hydride (1.32 g, 33.1 mmol, 60% dispersion). After 25 minutes, 5-bromo-2-cyanopyridine was added. After stirring the resulting mixture at room temperature for 20 minutes, the reaction was quenched by adding water (100 mL). The resulting mixture was extracted with ethyl acetate (200 mL). The organic layer was washed with water (4×) followed by saturated sodium chloride (1×). The organic layer was dried over anhydrous sodium sulfate, filtered and concentrated in vacuo. The residue was titurated with ethyl acetate (50 mL) to give the desired product as a white crystalline solid which was collected by filtration.

### Step B

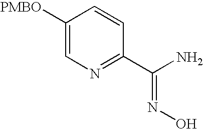

To a suspension of the intermediate from step A (5.0 g, 20.83 mmol) in ethanol (120 mL) was added hydroxylamine hydrochloride (1.74 g, 25 mmol) followed by NaOH (5 mL, 5N). After stirring the resulting slurry for 18 hours, it was filtered. The precipitate was washed with cold ethanol and dried under vacuum to give the desired product as a white crystalline solid.

### Step C

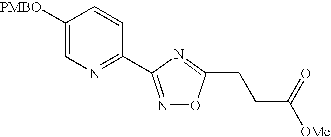

To a suspension of the intermediate from step B (5.0 g, 18.3 mmol) in anhydrous pyridine (15 mL) was added 4-chloro-4-oxo-methyl butyrate (2.68 mL, 21.97 mmol). The resulting reaction mixture was heated at 120° C. for 3 hours. The reaction was cooled to room temperature and concentrated in vacuo. The residue was dissolved in dichloromethane and washed with water (4×). The organic layer was dried over anhydrous sodium sulfate, filtered and concentrated in vacuo to give a dark brown solid. This material was titurated with methanol to give the desired product as a light tan solid.

### Step D

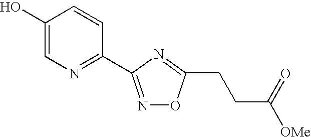

To a solution of the intermediate from step C (1.0 g, 2.71 mmol) in DCM (50 mL) was added TFA (20 mL). After stirring the reaction at room temperature for 30 minutes, it was concentrated in vacuo. The residue was suspended with ethyl acetate and washed with saturated sodium bicarbonate solution (3×). The organic layer was dried over anhydrous sodium sulfate, filtered and concentrated in vacuo.

### Step E

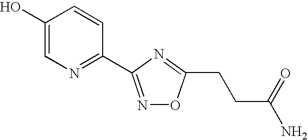

To a solution of the intermediate from step D (0.5 g, 2 mmol) in dioxane (10 mL) placed in a pressure vessel was added concentrated ammonium hydroxide solution (14 N, 50 mL). The resulting mixture stirred at 50° C. for 18 hours. The reaction was cooled to room temperature and concentrated in vacuo. The residue was extracted with ethyl acetate (3×), dried over anhydrous sodium sulfate, filtered and concentrated in vacuo to give a white solid.

### Step F

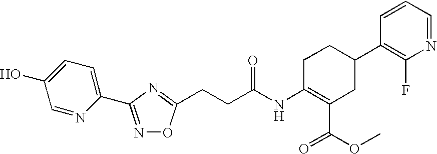

To a solution of the intermediate from example 6 step E (80 mg, 0.21 mmol) and the intermediate from step E (73 mg, 0.18 mmol) in dioxane (2 mL) was added DMF (0.5 mL), Xantphos (24 mg, 0.04 mmol), and cesium carbonate (81 mg, 0.25 mmol). The reaction mixture was flushed with N₂ and Pd₂(dba)₃ (19 mg, 0.02 mmol) was added. After stirring at 50° C. for 1 hour the reaction was cooled to room temperature, diluted with EtOAc and filtered through celite. The filtrate was concentrated in vacuo and purified by reverse phase HPLC (Gilson) 50% CH₃CN/H₂0-100% CH₃CN to give the desired product.

### Step G

To a solution of the intermediate from step F in dioxane (4 mL) was added MeOH (4 mL) followed by 1N LiOH (1.5 mL). After string at room temperature for 4 hours, the reaction mixture was neutralized by the addition of 1N HCl (115 mL). The reaction mixture was concentrated in vacuo and purified by reverse phase HPLC (Gilson) to give the desired product. ¹H NMR δ (500 MHz, DMSO) 8.27 (d, 1H), 7.92 (d, 1H), 7.36-7.31 (m, 2H), 7.12-7.09 (m, 2H), 7.02 (m, 1H), 3.21 (t, 2H), 3.07 (m, 1H), 2.94 (m, 2H), 2.87 (m, 1H), 2.75 (m, 1H), 2.59 (m, 1H), 2.24 (m, 1H), 1.87 (m, 1H), 1.70 (m, 1H). LCMS m/z 454 (M+1).

### Example 39

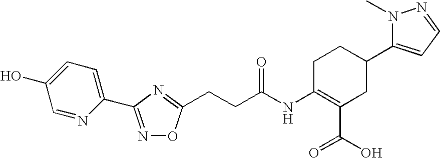

### Step A

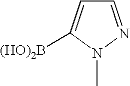

To a solution of N-methylpyrazole (2.0 g, 24.4 mmol) in anhydrous THF (100 mL) cooled to −78° C. under a N₂ atmosphere was added n-Butyl lithium (16.8 mL, 26.82 mmol, 1.6 M in hexanes). After 30 minutes, triisopropyl borate (6.75 mL, 29.27 mmol) was added. The reaction mixture was slowly warmed to 0° C. over 1 hour and then quenched with 1N HCl. The resulting mixture was stirred vigorously for 1 hour. The resulting mixture was extracted with ethyl acetate (3×). The organic layer was washed with brine, dried over anhydrous sodium sulfate, filtered and concentrated in vacuo to give the desired compound as a white solid.

### Step B

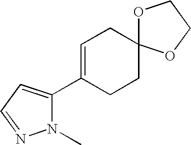

To a solution of the intermediate from step A (1.34 g, 1.2 equiv.) in 2:1 THF/2M Na₂CO₃ (75 mL total) was added the intermediate from example 6 step A (2.56 g, 1.0 equiv.). The resulting mixture was flushed with nitrogen and tetrakis triphenyl phosphine palladium (0) (513 mg, 5 mol %) was added. After stirring the reaction at 40° C. for 1 hour it was cooled to room temperature and neutralized with 1NHCl. The resulting mixture was extracted with EtOAc, washed with brine and dried over Na₂SO₄. The organic layer was filtered, concentrated in vacuo and purified by flash chromatography (silica-gel) using 1:1EtOAc/Hexto give a yellow oil.

### Step C

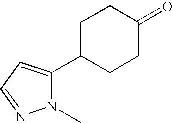

To a solution of the intermediate from step B (1.65 g, 7.5 mmol) in MeOH (100 mL) was added Pd/C (2 spatula's full) and stirred under H₂ balloon for 3 days. The reaction mixture was filtered through celite and concentrated yielding a white solid. This material (1.5 g, 6.75 mmol) was dissolved in acetone (60 mL), PPTS (1.59 g, 6.75 mmol) was added and the reaction heated to reflux for 3 days. The reaction mixture was concentrated in vacuo and re-dissolved in EtOAc. The organic layer was washed with saturated NaHCO₃, brine, dried over Na₂SO₄, filtered and concentrated. The residue was purified by flash chromatography (Biotage Horizon) using 50-100% EtOAc/Hex yielding a white solid.

### Step D

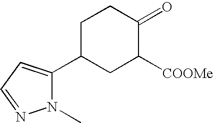

To a solution of the intermediate from step C (710 mg, 4 mmol) in anhydrous THF (30 mL) cooled to −78° C. under a nitrogen atmosphere was added LHMDS (1M, 4.38 mL, 1.1 equiv.). After stirring the reaction for 45 minutes at −78° C. methyl cyanoformate (0.347 mL, 4.38 mmol) was added. The reaction was warmed to −20° C. over 2 hours and then quenched by addition of 1N HCl. The reaction mixture was extracted with EtOAc (2×). The organic layer was washed with brine and dried over Na₂SO₄. The organic layer was filtered and concentrated in vacuo to give the desired product as an orange oil.

### Step E

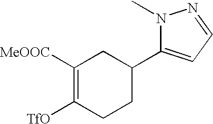

To solution of the intermediate from step D (1.04 g, 4.4 mmol) in THF (40 mL) at 0° C. was added NaH (60%, 211 mg, 1.2 equiv.). After stirring the reaction at room temperature for 30 minutes, 2-[N,N-Bis(trifluoromethylsulfonyl)amino]-5-chloropyridine was added (1.75 g, 4.4 mmol) was added. After stirring the reaction for 18 hours it was quenched by addition of water. The resulting mixture was extracted with EtOAc, washed with brine, and dried over Na₂SO₄. Crude product was purified by flash chromatography (Biotage, Horizon) using 50-70% EtOAc/Hex, yielding the desired product as a yellow oil.

### Step F

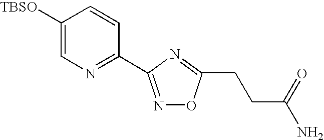

To a suspension of the intermediate from example 38 step E (0.5 g, 2 mmol) in DCM (50 mL) was added imidazole (204 mg, 3 mmol) followed by TBS-Cl (362 mg, 2.4 mmol). The resulting reaction was stirred at room temperature for 16 hours. The reaction mixture was poured into water and extracted with DCM (3×). The organic layer was dried over anhydrous sodium sulfate, filtered and concentrated in vacuo. The residue was purified by flash chromatography using 50% ethyl acetate-hexanes to give the desired product as a white solid.

### Step G

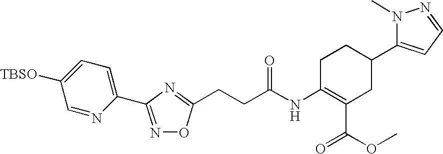

To a solution of the intermediate from step E (77 mg, 0.21 mmol) and the intermediate from step F (75 mg, 0.21 mmol) in dioxane (1.5 mL) was added Xantphos (20 mg, 0.034 mmol), and cesium carbonate (68 mg, 0.2 mmol). The reaction was flushed with N₂ and Pd₂(dba)₃ (11 mg, 0.012 mmol) was added. After stirring at 60° C. overnight, the reaction was cooled to room temperature, diluted with EtOAc and filtered through celite. The filtrate was concentrated in vacuo and purified by Prep-TLC (100% EtOAc/Hex) yielding the desired product.

### Step H

To a solution of the intermediate from step G (20 mg) in THF (2 mL) was added 1N NaOH (1.0 mL) and MeOH (0.5 mL). After stirring the reaction at room temperature overnight it was acidified to pH 6 by the addition of 1N HCl. The resulting mixture was extracted with 30% IPA/CHCl₃. The organic layer was washed with brine, dried over Na₂SO₄ and concentrated. The residue was purified by reverse-phase Gilson (10-70% CH₃CN/H₂O to give the desired compound. ¹H NMR (CD₃OD, 500 MHz), δ 8.23 (s, 1H), 8.05-8.03 (d, J=8.7 Hz, 1H) 7.45 (s, 1H), 7.40-7.38 (dd, J=8.4, 2.5 Hz, 1H), 6.17 (s, 1H), 3.84 (s, 3H), 3.27 (m, 2H), 3.16-3.12 (d, J=18.5 Hz, 1H), 3.02-2.99 (m, 4H), 2.81-2.77 (dd, J=17.2, 4.3 Hz, 1H), 2.36-2.33 (m, 1H), 2.03-2.00 (m, 1H), 1.7-1.66 (m, 1H). LCMS m/z 439 (M+1).

### Example 40

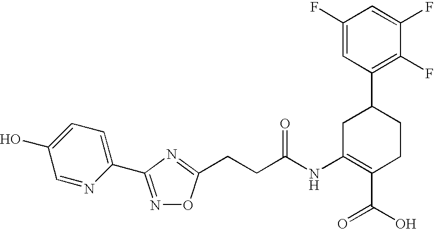

### Step A

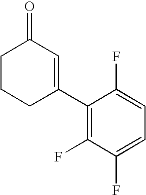

To a solution of the triflate from example 3 step A (8.71 g, 35.7 mmol) in THF (100 mL) was added 2,3,5-trifluorophenyl boronic acid, Na₂CO₃ (50 mL, 2.0 M solution) and dichlorobis(triphenylphosphine)palladium (1.0 g). The resulting mixture was heated at 60° C. under a nitrogen atmosphere. After 30 minutes, the reaction was cooled to room temperature and diluted with ethyl acetate. The organic layer was washed with brine, dried over anhydrous sodium sulfate, filtered and concentrated in vacuo. The residue was purified by flash chromatography using 10% ethyl acetate hexanes to give the desired compound as a light yellow/solid.

### Step B

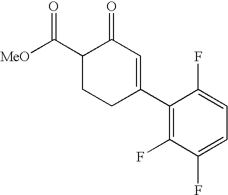

To a solution of the intermediate from step A (7.5 g, 33.15 mmol) in anhydrous THF cooled to −78° C. under a nitrogen atmosphere was added LHMDS (36.5 mL, 36.5 mmol, 1.0 M in THF). The reaction mixture was stirred at 0° C. for 25 minutes. It was then cooled to −78° C. and methyl cyano formate (3.16 mL, 39.78 mmol) was added. After 30 minutes, the reaction was quenched by pouring into water (100 mL). The resulting mixture was extracted with ethyl acetate (3×). The organic layer was washed with brine dried over anhydrous sodium sulfate, filtered and concentrated in vacuo. The residue was purified by flash chromatography (silica-gel) using 10% ethyl acetate-hexanes to give the desired product as a yellow solid.

### Step C

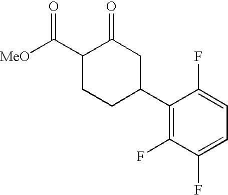

To a solution of the intermediate from step B (7.49 g, 26.37 mmol) in methanol (100 mL) was added Pd/C (100 mg, 10% by weight). The resulting reaction was stirred under H₂ balloon for 18 hours. The reaction mixture was filtered through celite. The filtrate was concentrated in vacuo and purified by flash chromatography using 10% ethyl acetate-hexanes to give the desired product as a colorless oil.

### Step D

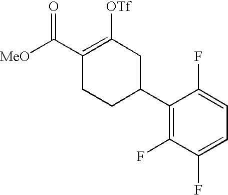

To a solution of the intermediate from step C (4.71 g, 16.47 mmol) in anhydrous THF (100 mL) cooled to 0° C. was added sodium hydride (0.99 g, 24.7 mmol, 60% dispersion). After 20 minutes, 2-[N,N-Bis(trifluoromethylsulfonyl)amino]-5-chloropyridine (7.76 g, 19.76 mmol) was added. The reaction was stirred at room temperature for 4 hours and then quenched with water. The resulting mixture was extracted with ethyl acetate (2×). The organic layer was washed with brine, dried over anhydrous sodium sulfate, filtered and concentrated in vacuo. The residue was purified by flash chromatography using 5% ethyl acetate-hexanes to give the desired product as a colorless oil.

### Step E

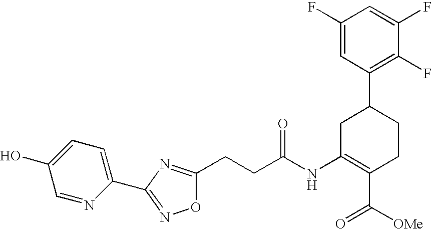

To a solution of the intermediate from step D (0.12 g, 0.28 mmol) and the intermediate from example 40 step F (0.085 g, 0.24 mmol) in anhydrous dioxane (3 mL) was added Xantphos (33 mg, 0.057 mmol) cesium carbonate (131 mg, 0.4 mmol) followed by Pd₂(dba)₃ (26 mg, 0.028 mmol). The resulting mixture was stirred under a nitrogen atmosphere at 60° C. for 3.5 hours. The reaction was cooled to room temperature and filtered through a pad of celite. The filtrate was concentrated in vacuo and purified by flash chromatography using 50% ethyl acetate-hexanes then 5% MeOH-ethyl acetate to give the desired product as a light yellow solid.

### Step F

To a solution of the intermediate from step E (75 mg, 0.12 mmol) in THF (2 mL) and MeOH (1 mL) was added 1N NaOH (1 mL). The resulting mixture was stirred at room temperature for 18 hours. The pH of the reaction was adjusted to pH=7 by the addition of 1N HCl (1 mL). The resulting mixture was concentrated in vacuo. The residue was diluted with water (5 mL) and extracted with ethyl acetate (3×). The organic layer was washed with brine, dried over anhydrous sodium sulfate, filtered and concentrated in vacuo. The residue was purified by reverse phase HPLC (Gilson) to give the desired product. ¹H NMR d (500 MHz DMSO) 11.66 (s, 1H), 10.62 (bs, 1H), 8.26 (d, J=2.3 Hz, 1H), 7.9 (d, J=8.4 Hz, 1H), 7.38 (m, 1H), 7.31 (m, 1H), 7.11 (m, 1H), 3.5 (m, 1H), 3.2 (m, 3H), 3.1 (bm, 1H), 2.95 (m, 2H), 2.8 (m, 1H), 2.35 (m, 1H), 1.8 (m, 2H). LCMS m/z 489 (M+1).

### Example 41

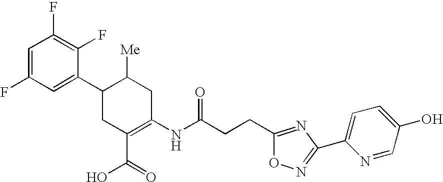

### Step A

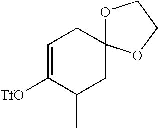

To a solution of ketone (4.0 g, 23.5 mmol) in anhydrous THF (100 mL) cooled to −78° C. under a N₂ atmosphere was added LHMDS (40 mL, 40 mmol, 1.0 M in THF). After stirring for 1 hour 2-[N,N-Bis(trifluoromethylsulfonyl)amino]-5-chloropyridine (10.0 g, 25.46 mmol) was added. The reaction was warmed to room temperature and stirred for 18 hours. The reaction was quenched with water and the resulting mixture was extracted with ethyl acetate (3×). The combined organic layers were washed with brine, dried over anhydrous sodium sulfate, filtered and concentrated in vacuo. The residue was purified by flash chromatography (Biotage, Horizon) using (0% EtOAc/Hexane→10% EtOAc/Hexane) to give the desired product as a pale orange oil.

### Step B

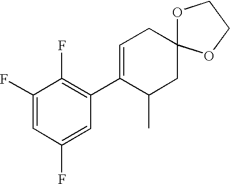

To a solution of the intermediate from step A (6.50 g, 24.29 mmol) in THF (200 mL) was added 2,3,5-trifluoroboronic acid (4.54 g, 25.8 mmol), and tetrakis triphenyl phosphine palladium (0) (1.00 g, 0.900 mmol). Aqueous sodium carbonate solution (1M, 43 mL) was added, the reaction mixture was flushed with N₂ and heated to 50° C. for 1 hour. The reaction was cooled to room temperature, diluted with ethyl acetate, washed with brine, and dried over sodium sulfate. The crude material was purified by flash chromatography (Bioatage Horizon) (0% EtOAc/Hexane→30% EtOAc/Hexane) to give the desired product.

### Step C

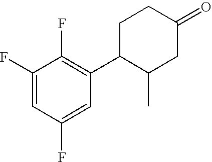

To a solution of the intermediate from step B (6.3 g, 21.9 mmol) in MeOH (150 mL) was added palladium on carbon (5%, 2 g) in MeOH (10 mL). The reaction mixture was stirred under a hydrogen balloon for 18 hours, and then filtered through celite and concentrated in vacuo. The crude material was dissolved in THF/EtOH (100 mL/40 mL) and HCl (20 mL, 3N) was added. The resulting mixture was stirred at room temperature for 18 hours. The reaction mixture was concentrated in vacuo.

The residue was diluted with ethyl acetate, and adjusted to pH=8 with saturated sodium bicarbonate. The resulting mixture was extracted with EtOAc (2×), washed with brine and dried over Na₂SO₄, filtered and concentrated in vacuo. The crude material was purified by flash chromatography (Biotage Horizon) (0% EtOAc/Hexane→30% EtOAc/Hexane) to give the desired product.

### Step D

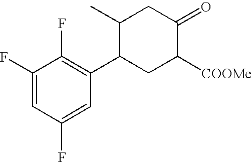

To a solution of the intermediate from step C (1.64 g, 6.66 mmol) in anhydrous THF (100 mL) cooled to −78° C. under a N₂ atmosphere was added LHMDS (8.00 mL, 8.00 mmol, 1.0 M in THF). After 30 min, methyl cyanoformate (0.695 mL, 8.66 mmol) was added and the reaction was allowed to warm to 0° C. over several hours. The reaction was quenched with 1N HCl and extracted with EtOAc (2×). The organic layer was washed with brine and dried over Na₂SO₄, filtered and concentrated in vacuo. This material was used in the next step without any further purification.

### Step E

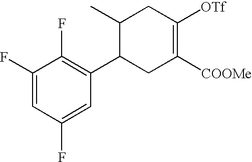

To a solution of the intermediate from step D (2.00 g, 6.66 mmol) in anhydrous THF (100 mL) was added NaH (399 mg, 9.99 mmol, 60%). After 15 minutes, a solution of 2-[N,N-Bis(trifluoromethylsulfonyl)amino]-5-chloropyridine (2.88 g, 7.33 mmol) in THF (20 mL) was added. The reaction was stirred at room temperature for 18 hours and then quenched with water. The resulting mixture was extracted with EtOAc (2×). The organic layer was washed with brine, dried over Na₂SO₄, filtered, and concentrated in vacuo. The crude material was purified by flash chromatography (Biotage, Horizon) (0% EtOAc/Hexane+20% EtOAc/Hexane) to give the desired product.

### Step F

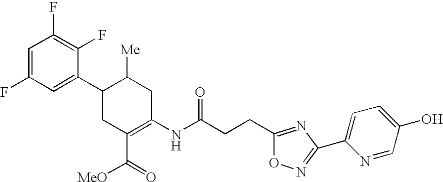

To a solution of the intermediate from step E (100 mg, 0.239 mmol) in anhydrous dioxane (2 mL) and DMF (0.5 mL) was added the amide (56 mg, 0.239 mmol), XANTPHOS (32 mg, 0.05 mmol), cesium carbonate (46 mg, 0.36 mmol) and Pd₂(dba)₃ (15 mg, 0.016 mmol). The resulting mixture was de-gassed for 2 minutes by bubbling N₂. The reaction was heated at 55° C. under a N₂ atmosphere for 18 hours. The reaction mixture was cooled to room temperature, and filtered through celite. The filtrate was concentrated in vacuo and the residue was purified by reverse phase HPLC (Gilson) to give the desired product.

### Step G

To a solution of the intermediate from step F in THF (2 mL) and MeOH (1 mL) was added 1N NaOH (1 mL). The resulting mixture was stirred at room temperature for 18 hours. The pH of the reaction was adjusted to pH=7 by the addition of 1N HCl (1 mL). The resulting mixture was concentrated in vacuo. The residue was diluted with water (5 mL) and extracted with ethyl acetate (3×). The organic layer was washed with brine, dried over anhydrous sodium sulfate, filtered and concentrated in vacuo. The residue was purified by reverse phase HPLC (Gilson) to give the desired product. ¹H NMR δ (500 MHz, DMSO) 8.26 (d, 1H), 7.90 (d, 1H), 7.38 (m, 1H), 7.30 (dd, 1H), 6.98 (m, 1H), 3.21 (t, 2H), 3.07 (m, 1H), 2.94 (m, 2H), 2.99-2.54 (m, 4H), 2.13 (m, 1H) 0.71 (d, 3H). LCMS m/z 503 (M+1).

### Example 42

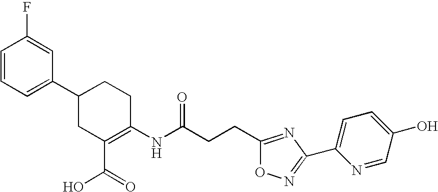

Example 42 was prepared following a similar procedure described for Example 38. ¹H NMR δ (500 MHz, DMSO) 1.94 (s, 1H), 8.11-8.04 (m, 2H), 7.84 (m, 1H), 7.48 (m, 1H), 7.27 (m, 1H), 3.29 (t, 2H), 3.17 (m, 1H), 3.03-3.97 (t, 3H), 2.78 (m, 1H), 2.53 (m, 1H), 2.38 (m, 1H), 1.98 (m, 1H), 1.83 (m, 1H). LCMS m/z 453 (M+1).

### Example 43

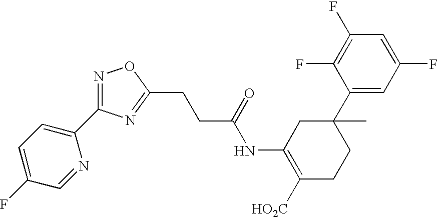

To a suspension of 5-amino-2-cyano pyridine (20-0 g, 0.168 mol) in HF-pyridine (100 g) in an Erlenmeyer flask cooled to 0° C. was added sodium nitrite (17.4 g, 0.251 mol) in four portions. After 45 min at 0° C. the reaction mixture was stirred at room temperature for 30 min and then heated to 80° C. for 90 min. The reaction mixture was quenched by pouring into an ice/water mixture. The resulting mixture was extracted with DCM. The organic layer was dried over anhydrous sodium sulfate, filtered and concentrated to give the fluoropyridine nitrile as an orange solid. To a suspension of this fluoropyridine nitrile intermediate (16.0 g, 0.131 mol) in methanol (200 mL) was added hydroxylamine (9.63 mL, 0.157 mmol, 50% by wt). After stirring the reaction mixture at room temperature for 48 h, it was filtered through a fritted funnel. The precipitate was washed with ether and dried under vacuum to give the N-hydroxyamidine as a yellow solid. To a suspension of this amidine intermediate (5.32 g, 34.32 mmol) in anhydrous pyridine (10 mL) was added 4-chloro-4-oxo-methyl butyrate (5 mL, 41.18 mmol). The resulting reaction mixture was heated at 120° C. for 2 h. The mixture was cooled to RT and concentrated. The residue was dissolved in ethyl acetate and washed with 1N HCl, water and brine. The organic layer was dried over anhydrous sodium sulfate, filtered and concentrated to give a dark brown solid. This material was purified by Biotage using 25%-60% ethyl acetate-hexanes gradient to give the heterobiaryl intermediate as a light yellow solid. To a solution of this ester intermediate (900 mg, 3.58 mmol) in dioxane (3 mL) was added ammonium hydroxide (3 mL) and the mixture was allowed to stir at room temperature for 12 hours. Upon completion, the mixture was concentrated, and the amide was purified via flash chromatography (Biotage 40M). To the amide (0.25 g, 1.0 mmol) in a degassed solution of dioxane (7 mL) was added the corresponding triflate (0.92 g, 2.1 mmol), cesium carbonate (1.0 g, 3.0 mmol), xantphos ligand (0.1 g, 0.2 mmol), and Pd₂(dba)₃ catalyst (0.09 g, 0.1 mmol), and the reaction mixture was heated to 75° C. for 6 hours. The mixture was cooled, filtered, concentrated in vacuo, and purified via flash chromatography (Biotage 40 M). To the desired cycloalkene (0.26 g, 0.5 mmol) in THF/H₂O (1:1) was added sodium hydroxide (0.06 g, 1.5 mmol). The biphasic reaction mixture was allowed to stir for 12 hours at room temperature. The mixture was concentrated in vacuo and purified by reverse phase HPLC (Gilson) to afford the desired product Example 43. ¹H NMR (DMSO-d₆, 500 MHz) δ 11.46 (s, 1H), 8.76 (s, 1H), 8.12 (m, 1H), 7.94 (m, 1H), 7.41 (m, 1H), 6.87 (m, 1H), 3.43 (m, 2H), 3.26 (m, 2H), 2.89 (m, 2H), 2.18 (m, 1H), 2.11 (m, 2H) 1.88, (m, 1H), 1.30 (m, 3H); LCMS m/z 527 (M+Na).

### Example 44

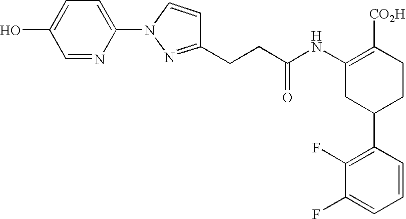

To a solution of ethyl-3-pyrazole carboxylate (3.53 g, 25.2 mmol) in DMF (40 mL) at 0° C. was added sodium hydride (60%, 1.21 g, 30.2 mmol). The resulting mixture was stirred at room temperature for 40 min followed by the addition of 5-nitro-2-bromopyridine (5.1 g, 25.2 mmol). After being stirred for 20 min, the reaction mixture was partitioned between dichloromethane (1000 mL) and water (500 mL), the organic phase was washed with water (3×500 mL), dried over sodium sulfate, and concentrated in vacuo. The residue was purified by flash chromatography using 80% DCM/hexane to give the desired product. To this nitro intermediate (6.77 g, 25.8 mmol) in acetic acid (220 mL) was added zinc powder (16.77 g, 258 mmol). The resulting mixture was heated at 60° C. for 30 min before it was filtered. The filtrate was concentrated in vacuum. To the residue was added DCM (1000 mL) and saturated sodium bicarbonate (1000 mL), and the resulting mixture was stirred at room temperature overnight. The organic phase was then washed with saturated sodium bicarbonate, dried over sodium sulfate, and concentrated in vacuo. The residue was purified by flash chromatography using 5% methanol in DCM (containing 0.1% triethylamine) to give the desired product as a yellow solid. To this amine intermediate (5.96 g, 25.7 mmol) in tetrafluoroboric acid (48%, 130 mL) at 0° C. was added a solution of sodium nitrite (1.95 g, 28.3 mmol) in water (20 mL) dropwise. The resulting solution was stirred at 0° C. for 1 h before filtration. The solid was washed with water and diethyl ether to give the desired product as a yellow solid. A mixture of this diazo intermediate (6.66 g) in acetic anhydride (250 mL) was heated at 70° C. overnight before it was concentrated in vacuo. The residue was purified by flash chromatography eluting with DCM to give the desired product as a white solid. A solution of this acetate intermediate (3.5 g, 12.7 mmol) in ethanol (400 mL) in the presence of 4 drops of sulfuric acid was heated under reflux overnight. After being concentrated in vacuo, the residue was partitioned between DCM (300 mL) and water (200 mL). The pH of the resulting mixture was adjusted to pH=5 by saturated sodium bicarbonate solution. The DCM phase was dried with sodium sulfate and concentrated in vacuo to give the product as a solid. To a solution of this hydroxyl intermediate (2.86 g, 12.3 mmol) in DMF (40 mL) at 0° C. was added sodium hydride (60%, 589 mg, 14.73 mmol). The resulting mixture was stirred at room temperature for 40 min followed by adding 4-methoxybenzyl alcohol (2.31 g, 14.73 mmol) and sodium iodide (10 mg). The resulting mixture was heated at 80° C. for 0.5 h. After being cooled to room temperature, the reaction mixture was partitioned between DCM (500 mL) and brine (500 mL). The DCM phase was washed with brine (3×500 mL), dried over sodium sulfate, and concentrated in vacuo. The residue was treated with 20% EtOAc/hexane (50 mL) and the mixture was filtered to give the desired product. The filtrate was concentrated and the resulting residue was purified by flash chromatography using 20% EtOAc/hexane to give additional product as a white solid. A suspension of this ethyl ester intermediate (4.13 g, 11.9 mmol) and lithium borohydride (384 mg, 17.6 mmol) in THF (300 mL) was heated under reflux overnight before it was cooled to 0° C. and quenched by 1N HCl until pH=6. The resulting mixture was diluted in EtOAc (400 mL) and washed with saturated sodium bicarbonate (2×400 mL), dried over sodium sulfate and concentrated in vacuo to give the desired product as a white solid. To a solution of this alcohol (3.7 g, 11.88 mmol) in DCM (200 mL) at 0° C. was added pyridine (1.13 g, 14.27 mmol), triphenylphosphine (8.73 g, 33.29 mmol) and NBS (6.34 g, 35.66 mmol). The resulting solution was stirred at 0° C. for 1.5 h. The DCM phase was washed with brine, dried over sodium sulfate and concentrated in vacuo. The residue was purified by flash chromatography eluting with DCM to give the product as a white solid. To a solution of dimethylmalonate (6.0 g, 45.6 mmol) in DMF (100 mL) at 0° C. was added sodium hydride (2.0 g, 50.15 mmol, 60%). The mixture was stirred at 0° C. for 40 min before addition of the bromide intermediate (3.41 g, 9.12 mmol) as one portion. The resulting mixture was stirred at room temperature for 40 min before it was partitioned between ethyl acetate (500 mL) and saturated ammonium chloride (300 mL). The EtOAc phase was washed with brine (3×500 mL), dried over sodium sulfate and concentrated in vacuo. The residue was purified by flash chromatography eluting with 20% EtOAc/hexane to give the product as a white solid. To a solution of this diester (3.8 g, 8.9 mmol) in a mixture solvents of THF/MeOH/H₂O (3:1:1, 300 mL) was added lithium hydroxide (1N, 150 mL) dropwise at room temperature. The solution was stirred for 40 min before it was concentrated in vacuo to remove the organic solvents. The resulting alkaline solution was then acidified to pH=3 by 3N HCl followed by extraction with EtOAc (2×300 mL). The combined EtOAc phases were washed with brine (3×300 mL), dried over sodium sulfate, and concentrated in vacuo to afford the product as a white solid. A solution of this diacid (3.28 g, 8.26 mmol) in DMF (40 mL) was heated at 130° C. for 20 min. After cooling to room temperature, the reaction mixture was partitioned between EtOAc (300 mL) and brine (300 mL). The EtOAc phase was washed with brine (2×300 mL), dried over sodium sulfate, and concentrated in vacuo to afford the product as a white solid. A solution of this monoacid intermediate (2.7 g, 7.7 mmol), N-hydroxysuccinimide (0.97 g, 8.4 mmol), EDCI (1.76 g, 9.2 mmol) in DCM (100 mL) was stirred at room temperature for 3 h before it was diluted by 400 mL DCM. The DCM phase was washed brine (3×300 mL), dried over sodium sulfate, and concentrated in vacuo to afford the product. To a solution of this residue in 1,4-dioxane (200 mL) was added ammonium hydroxide (28%, 30 mL) dropwise at room temperature. The resulting mixture was stirred for 30 min before it was neutralized by concentrated HCl. The mixture was then concentrated in vacuo. The residue was partitioned between DCM (500 mL) and water (500 mL). The aqueous phase was extracted with DCM (3×200 mL). The combined DCM phase was then washed with saturated sodium bicarbonate (3×500 mL), dried over sodium sulfate, and concentrated in vacuo to afford the carboxamide common intermediate as a white solid (Intermediate 84 illustrated in Scheme 14). In parallel, to a solution of 1,3-cyclohexanedione (6.0 g, 53.5 mmol) in DCM (250 mL), at −78° C., was added 2,6-lutidine (8.6 g, 80.3 mmol) and trifluoroacetic anhydride (18.1 g, 64.2 mmol) dropwise. The resulting solution was stirred at room temperature for 1 h before it was washed with hydrochloric acid (1N, 3×100 mL). The DCM phase was dried over sodium sulfate and concentrated in vacuo to afford the product as a red brown oil. A mixture of this triflate (4.0 g, 16.39 mmol), 2,3-difluorophenylboronic acid (3.11 g, 19.67 mmol), bis(triphenylphosphine)dichloride palladium (11) (0.5 g, 0.71 mmol), and sodium carbonate (2M, 40 mL) in THF (100 mL) was flushed with nitrogen before it was stirred at room temperature overnight. The reaction mixture was quenched with water (200 mL) and washed with EtOAc (3×300 mL). The combined EtOAc phase was washed with brine (3×300 mL), dried over sodium sulfate, and concentrated in vacuo. The resulting residue was purified by flash column chromatography eluting with 10% EtOAc/hexane to afford the product as a white solid. To a solution of this intermediate (2.94 g, 14.1 mmol) in THF (100 mL) at −78° C. was added lithium bis(trimethylsilyl)amide (15.5 mL, 1N in TH) dropwise. After the resulting solution was stirred at 0° C. for 30 min, to this solution was added methyl cyanoformate (1.32 g, 15.5 mmol) dropwise at −78° C. The resulting solution was then stirred at −20° C. for 2 h before it was quenched by HCl (1N) until pH=4. The mixture was extracted with EtOAc (200 mL) and the EtOAc phase was washed with brine (3×200 mL), dried over sodium sulfate, and concentrated in vacuo. The resulting residue was purified by flash chromatography eluting with 10% EtOAc/hexane to afford the product as an oil. This enone (2.15 g, 8.1 mmol) was subjected to hydrogenation in methanol (150 mL) in the presence of palladium/carbon (5%, 0.43 g) at room temperature under a hydrogen balloon for 1.5 h before it was filtered under nitrogen atmosphere through celite. The filtrate was concentrated in vacuo. The residue was purified by flash chromatography eluting with 8% EtOAc/hexane to afford the product as a solid. To a solution of this beta-ketoester (0.38 g, 1.43 mmol) in THF (20 mL) at 0° C. was added sodium hydride (74.3 mg, 1.85 mmol, 60%). After the resulting mixture was stirred at room temperature for 20 min, 2-[N,N-bis(trifluoromethylsulfonyl)amino]-5-chloropyridine (0.59 g, 1.5 mmol) was added to the above mixture, and the resulting mixture was stirred at room temperature for 1.5 h before the addition of water (100 mL). The mixture was extracted with EtOAc (200 mL). The EtOAc phase was washed with brine (3×200 mL), dried over sodium sulfate, and concentrated in vacuo. The residue was purified by flash chromatography eluting with 5% EtOAc/hexane to afford the triflate as an oil. A mixture of the previous carboxamide common intermediate (0.22 g, 0.62 mmol), tri(dibenzylideneacetone)dipalladium (0) (57 mg, 0.062 mmol), 4,5-bis(diphenylphosphino)-9,9-dimethylxanthene (91 mg, 0.16 mmol), the triflate (0.31 g, 0.78 mmol), and cesium carbonate (0.41 g, 1.25 mmol) in 1,4-dioxane (40 mL) was heated under argon at 80° C. overnight. The reaction mixture was filtered through celite, and the filtrate was concentrated in vacuo. The residue was purified by flash chromatography eluting with 20% EtOAc/hexane to afford the product amide as an oil. To a solution of this intermediate (0.17 g, 0.28 mmol) and triisopropylsilane (0.11 g, 0.70 mmol) in DCM (1.4 mL) at 0° C. was added trifluoroacetic acid (0.7 mL). The resulting solution was stirred at room temperature for 20 min before it was concentrated in vacuo below 12° C. The residue was dissolved in a mixed solvent of THF/MeOH/H₂O (3:1:1, 20 mL) at 0° C. To the above solution was added lithium hydroxide (10 mL, 1N) dropwise. The resulting mixture was stirred at room temperature overnight. After removing the organic solvents in vacuo, the aqueous solution was acidified by 1N HCl to pH=4 followed by extracting the mixture with isopropanol/chloroform (30%, 2×40 mL). The combined organic phases was then concentrated in vacuo. The residue was purified on preparative RP-HPLC to afford the desired product Example 44. LCMS: 469 (M+1), ¹H NMR (500 MHz, DMSO-d₆): 11.66 (1H, s), 10.02 (1H, s), 8.32 (1H, d), 7.94 (1H, d), 7.67 (1H, d), 7.30 (2H, m), 7.17 (2H, m), 6.32 (1H, d), 2.88 (2H, t), 2-67 (2H, t), 3.16 (1H, m), 2.94 (1H, m), 2.58 (1H, m), 2.43 (1H, m), 1.94 (1H, m), 1.81 (1H, m).

### Example 45

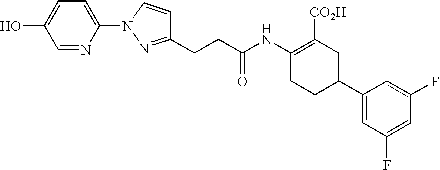

To a solution of 1,4-dioxaspiro[4,5]decan-8-one (8.0 g, 51.2 mmol) in THF (180 mL) at −78° C. was added lithium bis(trimethylsilyl)amide (56.4 mL, 1N) dropwise. After the resulting mixture was stirred at room temperature for 20 min, 2-[N,N-bis(trifluoromethylsulfonyl)amino]-5-chloropyridine (20.0 g, 51.2 mmol) was added to the above mixture, and the resulting mixture was stirred at room temperature for 1.5 h before the reaction was quenched by water (200 mL). The mixture was extracted with EtOAc (300 mL). The EtOAc phase was washed with brine (3×200 mL), dried over sodium sulfate, concentrated in vacuo. The residue was purified by flash chromatography eluting with 10% EtOAc/hexane to afford the triflate as an oil. A mixture of this intermediate (5.0 g, 16.6 mmol), 3,5-difluorophenylboronic acid (3.14 g, 19.9 mmol), bis(triphenylphosphine)dichloride palladium (II) (0.91 g, 1.0 mmol), and sodium carbonate (2M, 40 mL) in THF (100 mL) was flushed with nitrogen before it was stirred at room temperature overnight. The reaction mixture was quenching by water (200 mL) and extracted with EtOAc (3×300 mL). The combined EtOAc phase was washed with brine (3×300 mL), dried over sodium sulfate, and concentrated in vacuo to afford the crude product as an oil. The crude intermediate was subjected to hydrogenation in methanol (100 mL) in the presence of palladium/carbon (5%, 0.35 g) at room temperature under a hydrogen balloon overnight before the reaction mixture was filtered under nitrogen atmosphere through celite. The filtrate was concentrated in vacuo to afford a crude solid. To a solution of this ketal intermediate in THF (100 mL) was added HCl (3N, 20 mL). The resulting mixture was stirred at room temperature for 5 h and then concentrated in vacuo. The acidic aqueous phase was extracted with EtOAc (2×100 mL). The combined EtOAc phase was washed with water (2×100 mL), saturated sodium bicarbonate (2×100 mL), brine (100 mL), and concentrated in vacuo. The residue was purified by flash chromatography eluting with 8% EtOAc/hexane to afford the product as a white solid. To a solution of this ketone (2.82 g, 13.4 mmol) in THF (40 mL) at −78° C. was added lithium bis(trimethylsilyl)amide (16.1 mL, 1N) dropwise. After the resulting solution was stirred at 0° C. for 30 min, to this solution was added methyl cyanoformate (1.61 g, 18.8 mmol) dropwise at −78° C. The resulting solution was then stirred at −20° C. for 2 h before the addition of HCl (1N) until pH=4. The mixture was extracted with EtOAc (200 mL), and the EtOAc phase was washed with brine (3×200 mL), dried over sodium sulfate, and concentrated in vacuo. The resulting residue was purified by flash chromatography eluting with 10% EtOAc/hexane to afford the product as an oil. To a solution of this beta-ketoester (1.47 g, 5.49 mmol) in THF (50 mL) at 0° C. was added sodium hydride (285 mg, 7.13 mmol, 60%). After the resulting mixture was stirred at room temperature for 20 min, 2-[N,N-bis(trifluoromethylsulfonyl)amino]-5-chloropyridine (2.37 g, 6.04 mmol) was added to the above mixture, and the resulting mixture was stirred at room temperature for 1.5 h before the addition of water (100 mL). The mixture was extracted with EtOAc (200 mL). The EtOAc phase was washed with brine (3×200 mL), dried over sodium sulfate, and concentrated in vacuo. The residue was purified by flash chromatography eluting with 5% EtOAc/hexane to afford the product as an oil. A mixture of the previous carboxamide common intermediate (0.22 g, 0.62 mmol), tris(dibenzylideneacetone)dipalladium (0) (57 mg, 0.062 mmol), 4,5-bisdiphenylphosphino)-9,9-dimethylxanthene (91 mg, 0.16 mmol), the triflate (0.50 g, 1.2 mmol), and cesium carbonate (0.41 g, 1.25 mmol) in 1,4-dioxane (40 mL) was heated under argon at 80° C. overnight. After being filtered through celite, the filtrate was concentrated in vacuo. The residue was purified by flash chromatography eluting with 20% EtOAc/hexane to afford the product as an oil. To a solution of this intermediate (0.18 g, 0.30 mmol) and triisopropylsilane (0.11 g, 0.70 mmol) in DCM (1.4 mL) at 0° C. was added trifluoroacetic acid (0.7 mL). The resulting solution was stirred at room temperature for 20 min before it was concentrated in vacuo below 12° C. The residue was dissolved in a mixed solvent of THF/MeOH/H₂O (3:1:1, 20 mL) at 0° C. To the above solution was added lithium hydroxide (10 mL, 1N) dropwise. The resulting mixture was stirred at room temperature overnight. After removing the organic solvents in vacuo, the aqueous solution was acidified by 1N HCl to pH=4 followed by extracting with isopropanol/chloroform (30%, 2×40 mL). The combined organic phase was then concentrated in vacuo. The residue was purified on preparative RP-HPLC to afford the desired product Example 45. LCMS: 469 (M+1), ¹H NMR (500 MHz, CD₃OD): 8.30 (1H, d), 7.94 (1H, d), 7.72 (1H, d), 7.33 (1H, q), 6.88 (2H, m), 6.76 (1H, m), 6.33 (1H, d), 3.32 (2H, t), 3.20 (1H, m), 2.95 (1H, m), 2.74 (2H, t), 2.80 (2H, m), 2.30 (1H, m), 1.97 (1H, m), 1.74 (1H, m).

### Biological Assays

The activity of the compounds of the present invention regarding niacin receptor affinity and function can be evaluated using the following assays:

³H-Niacin binding assay:

1. Membrane: Membrane preps are stored in liquid nitrogen in:
20 mM HEPES, pH 7.4
0.1 mM EDTA

Thaw receptor membranes quickly and place on ice. Re-suspend by pipetting up and down vigorously, pool all tubes, and mix well. Use clean human at 15 μg/well, clean mouse at 10 μg/well, dirty preps at 30 μg/well.
1a. (human): Dilute in Binding Buffer.
1b. (human+4% serum): Add 5.7% of 100% human serum stock (stored at −20° C.) for a final concentration of 4%. Dilute in Binding Buffer.
1c. (mouse): Dilute in Binding Buffer.

2. Wash buffer and dilution buffer: Make 10 liters of ice-cold Binding Buffer:
20 mM HEPES, pH 7.4
1 mM MgCl₂
0.01% CHAPS (w/v)
use molecular grade or ddH₂O water

3. [5, 6-3H]-nicotinic acid: American Radiolabeled Chemicals, Inc. (cat #ART-689). Stock is ˜50 Ci/mmol, 1 mCi/ml, 1 ml total in ethanol→20 μM

Make an intermediate ³H-niacin working solution containing 7.5% EtOH and 0.25 μM tracer. 40 μL of this will be diluted into 200 μL total in each well→1.5% EtOH, 50 nM tracer final.

4. Unlabeled nicotinic acid:

Make 100 mM, 10 mM, and 80 μM stocks; store at −20° C. Dilute in DMSO.

5. Preparing Plates:
1) Aliquot manually into plates. All compounds are tested in duplicate. 10 mM unlabeled nicotinic acid must be included as a sample compound in each experiment.
2) Dilute the 10 mM compounds across the plate in 1:5 dilutions (8 μl:40 μl).
3) Add 195 μL binding buffer to all wells of Intermediate Plates to create working solutions (250 μM→0). There will be one Intermediate Plate for each Drug Plate.
4) Transfer 5 μL from Drug Plate to the Intermediate Plate. Mix 4-5 times.

6. Procedure:
1) Add 140 μL of appropriate diluted 19CD membrane to every well. There will be three plates for each drug plate: one human, one human+serum, one mouse.
2) Add 20 μL of compound from the appropriate intermediate plate
3) Add 40 μL of 0.25 μM 3H-nicotinic acid to all wells.
4) Seal plates, cover with aluminum foil, and shake at RT for 34 hours, speed 2, titer plate shaker.
5) Filter and wash with 8×200 μL ice-cold binding buffer. Be sure to rinse the apparatus with >1 liter of water after last plate.
6) Air dry overnight in hood (prop plate up so that air can flow through).
7) Seal the back of the plate
8) Add 40 μL Microscint-20 to each well.
9) Seal tops with sealer.
10) Count in Packard Topcount scintillation counter.
11) Upload data to calculation program, and also plot raw counts in Prism, determining that the graphs generated, and the IC₅₀ values agree.

The compounds of the invention generally have an IC₅₀ in the ³H-nicotinic acid competition binding assay within the range of 1 nM to about 25 μM.

### ³⁵S-GTPγS Binding Assay:

Membranes prepared from Chinese Hamster Ovary (CHO)-K1 cells stably expressing the niacin receptor or vector control (7 μg/assay) were diluted in assay buffer (100 mM HEPES, 100 mM NaCl and 10 mM MgCl₂, pH 7.4) in Wallac Scintistrip plates and pre-incubated with test compounds diluted in assay buffer containing 40 μM GDP (final [GDP] was 10 μM) for ˜10 minutes before addition of ³⁵S-GTPγS to 0.3 nM. To avoid potential compound precipitation, all compounds were first prepared in 100% DMSO and then diluted with assay buffer resulting in a final concentration of 3% DMSO in the assay. Binding was allowed to proceed for one hour before centrifuging the plates at 4000 rpm for 15 minutes at room temperature and subsequent counting in a TopCount scintillation counter. Non-linear regression analysis of the binding curves was performed in GraphPad Prism.

### Membrane Preparation

### Materials:

CHO-K1 cell culture medium: F-12 Kaighn's Modified Cell Culture Medium with 10% FBS, 2 mM L-Glutamine, 1 mM Sodium Pyruvate and 400 μg/ml G418

### Membrane Scrape Buffer:

20 mM HEPES
10 mM EDTA, pH 7.4

### Membrane Wash Buffer:

20 mM HEPES
0.1 mM EDTA, pH 7.4
   Protease Inhibitor Cocktail: P-8340, (Sigma, St. Louis, Mo.)

### Procedure:

(Keep everything on ice throughout prep; buffers and plates of cells)
Aspirate cell culture media off the 15 cm² plates, rinse with 5 mL cold PBS and aspirate.
Add 5 ml Membrane Scrape Buffer and scrape cells. Transfer scrape into 50 mL centrifuge tube. Add 50 uL Protease Inhibitor Cocktail.
Spin at 20,000 rpm for 17 minutes at 4° C.
Aspirate off the supernatant and resuspend pellet in 30 mL Membrane Wash Buffer. Add 50 μL Protease Inhibitor Cocktail.
Spin at 20,000 rpm for 17 minutes at 4° C.
Aspirate the supernatant off the membrane pellet. The pellet may be frozen at −80° C. for later use or it can be used immediately.

### Assay

### Materials:

Guanosine 5′-diphosphate sodium salt (GDP, Sigma-Aldrich Catalog #87127)
Guanosine 5′-[γ³⁵S] thiotriphosphate, triethylammonium salt ([³⁵S]GTPγS, Amersham Biosciences Catalog #SJ1320, ˜1000 Ci/mmol)
96 well Scintiplates (Perkin-Elmer #1450-501)
Binding Buffer:
   20 mM HEPES, pH 7.4
   100 mM NaCl
   10 mM MgCl₂
GDP Buffer: binding buffer plus GDP, ranging from 0.4 to 40 μM, make fresh before assay

### Procedure:

(total assay volume=100 swell)

25 μL GDP buffer with or without compounds (final GDP 10 μM—so use 40 μM stock)

50 μL membrane in binding buffer (0.4 mg protein/mL)

25 μL [³⁵S]GTP-γS in binding buffer. This is made by adding 5 μl [³⁵S]GTPγS stock into 10 mL binding buffer (This buffer has no GDP)
Thaw compound plates to be screened (daughter plates with 5 L compound @ 2 mM in 100% DMSO)
Dilute the 2 mM compounds 1:50 with 245 μL GDP buffer to 40 μM in 2% DMSO. (Note: the concentration of GDP in the GDP buffer depends on the receptor and should be optimized to obtain maximal signal to noise; 40 μM).
Thaw frozen membrane pellet on ice. (Note: they are really membranes at this point, the cells were broken in the hypotonic buffer without any salt during the membrane prep step, and most cellular proteins were washed away)
Homogenize membranes briefly (few seconds—don't allow the membranes to warm up, so keep on ice between bursts of homogenization) until in suspension using a POLYTRON PT3100 (probe PT-DA 3007/2 at setting of 7000 rpm). Determine the membrane protein concentration by Bradford assay. Dilute membrane to a protein concentrations of 0.40 mg/ml in Binding Buffer. (Note: the final assay concentration is 20 μg/well).
Add 25 μL compounds in GDP buffer per well to Scintiplate.
Add 50 μL of membranes per well to Scintiplate.
Pre-incubate for 5-10 minutes at room temperature. (cover plates with foil since compounds may be light sensitive)
Add 25 μL of diluted [³⁵S]GTPγS. Incubate on shaker (Lab-Line model #1314, shake at setting of 4) for 60 minutes at room temperature. Cover the plates with foil since some compounds might be light sensitive.
Assay is stopped by spinning plates' sealed with plate covers at 2500 rpm for 20 minutes at 22° C.
Read on TopCount NXT scintillation counter—35S protocol.

The compounds of the invention generally have an EC₅₀ in the functional in vitro GTPγS binding assay within the range of about 10 nM to as high as about 100 μM.

### Flushing Via Laser Doppler

Male C57B16 mice (˜25 g) are anesthetized using 10 mg/ml/kg Nembutal sodium. When antagonists are to be administered they are co-injected with the Nembutal anesthesia. After ten minutes the animal is placed under the laser and the ear is folded back to expose the ventral side. The laser is positioned in the center of the ear and focused to an intensity of 8.4-9.0 V (with is generally ˜4.5 cm above the ear). Data acquisition is initiated with a 15 by 15 image format, auto interval, 60 images and a 20 sec time delay with a medium resolution. Test compounds are administered following the 10th image via injection into the peritoneal space. Images 1-10 are considered the animal's baseline and data is normalized to an average of the baseline mean intensities.

Materials and Methods—Laser Doppler Pirimed PimII; Niacin (Sigma); Nembutal (Abbott labs).

All patents, patent applications and publications that are cited herein are hereby incorporated by reference in their entirety. While certain preferred embodiments have been described herein in detail, numerous alternative embodiments are seen as falling within the scope of the invention.

What is claimed is:

1. A compound represented by formula I: 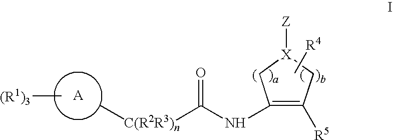
or a pharmaceutically acceptable salt or solvate thereof is disclosed wherein:
X represents a carbon or nitrogen atom;
Z represents Aryl and Heteroaryl, said Aryl and Heteroaryl being optionally substituted with 1-3 groups, 1-3 of which are halo, and 0-1 of which are selected from the group consisting of: OH, NH₂, C₁₋₃alkyl, C₁₋₃alkoxy, haloC₁₋₃alkyl and haloC₁₋₃alkoxy groups;
R⁴ is H, fluoro, or C₁₋₃alkyl optionally substituted with 1-3 groups, 0-3 of which are halo, and 0-1 of which are selected from the group consisting of: OC₁₋₃alkyl, OH, NH₂, NHC₁₋₃alkyl, N(C₁₋₃alkyl)₂, CN and Hetcy;
a and b are each integers 1 or 2, such that the sum of a and b is 3;
ring A represents a 6-10 membered Aryl, or a 5-13 membered Heteroaryl group, said Heteroaryl group containing at least one heteroatom selected from O, S, S(O), S(O)₂ and N, and optionally containing 1 other heteroatom selected from O and S, and optionally containing 1-3 additional N atoms, with up to 5 heteroatoms being present;
each R² and R³ is independently H, C₁₋₃alkyl, haloC₁₋₃alkyl, OC₁₋₃alkyl, haloC₁₋₃alkoxy, OH or F;
n represents an integer of from 2 to 4;
R⁵ represents —CO₂H, 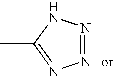
—C(O)NHSO₂R^{e} wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;
and each R¹ is H or is independently selected from the group consisting of:
a) halo, OH, CO₂H, CN, NH₂, S(O)₀₋₂R^{e}, C(O)R^{e}, OC(O)R^{e} and CO₂R^{e}, wherein R^{e} is as previously defined;
b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;
c) NHC₁₋₄alkyl and N(C₁₋₄alkyl)₂, the alkyl portions of which are optionally substituted as set forth in (b) above;
d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;
e) NR′C(O)R″, NR′SO₂R″, NR′CO₂R″ and NR′C(O)NR″R′″ wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₁₋₄alkoxy groups;
(b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;
and R′″ representing H or R″;
f) phenyl or a 5-6 membered Heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above.

2. A compound in accordance with claim 1 wherein ring A represents an Aryl group, a 5-6 membered monocyclic Heteroaryl group or a 9-13 membered bicyclic or tricyclic Heteroaryl group.

3. A compound in accordance with claim 2 wherein: ring A is selected from the group consisting of:
a) Aryl selected from phenyl and naphthyl;
b) HAR selected from the group consisting of: pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, benzopyrazolyl, benzotriazolyl, furo(2,3-b)pyridyl, benzoxazinyl, tetrahydrohydroquinolinyl, tetrahydroisoquinolinyl, quinolyl, isoquinolyl, indolyl, dihydroindolyl, quinoxalinyl, quinazolinyl, naphthyridinyl, pteridinyl, 2,3-dihydrofuro(2,3-b)pyridyl indolinyl, dihydrobenzofuranyl, dihydrobenzothiophenyl, dihydrobenzoxazolyl, or a member selected from the group consisting of:

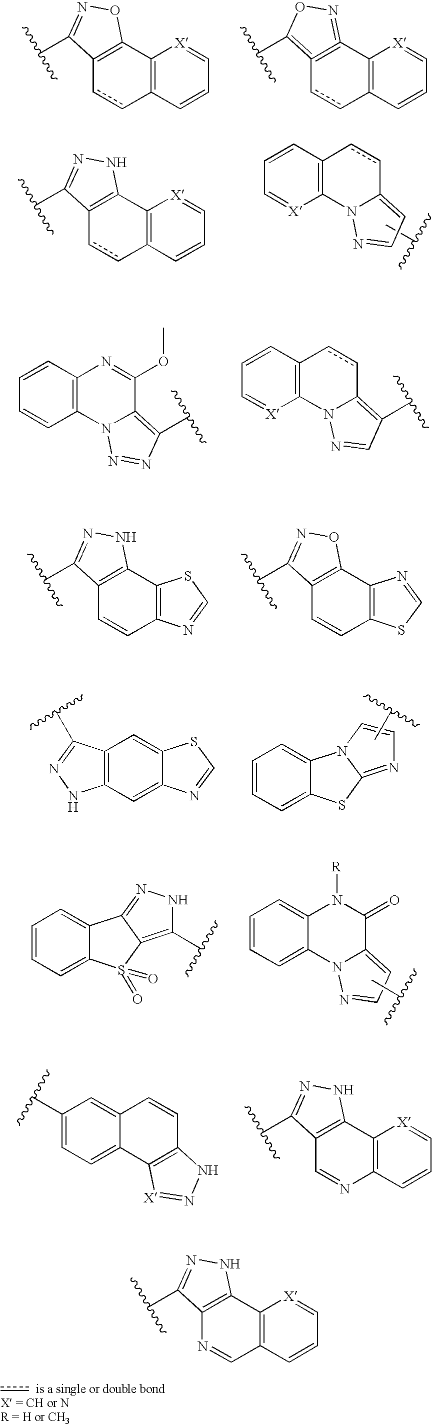

4. A compound in accordance with claim 3 wherein ring A is selected from the group consisting of: phenyl, naphthyl, pyrrolyl, isoxazolyl, isothiazolyl, pyrazolyl, pyridyl, oxazolyl, oxadiazolyl, thiadiazolyl, thiazolyl, imidazolyl, triazolyl, furanyl, and thienyl.

5. A compound in accordance with claim 4 wherein ring A is selected from the group consisting of: phenyl, naphthyl, oxadiazolyl, pyrazolyl and thiazolyl.

6. A compound in accordance with claim 1 wherein each R¹ is H or is independently selected from the group consisting of:
a) halo, OH, CO₂H, CN, NH₂, S(O)₀₋₂R^{e}, C(O)R^{e}, OC(O)R^{e} and CO₂R^{e}, wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;
b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;
c) phenyl or a 5-6 membered Heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″;
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₁₋₄alkoxy groups;
(b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;
and R′″ representing H or R″.

7. A compound in accordance with claim 6 wherein each R¹ is H or is independently selected from the group consisting of:
a) halo, OH, CO₂H, CN, NH₂, S(O)₀₋₂R^{e}, C(O)R^{e}, OC(O)R^{e} and CO₂R^{e}, and
b) phenyl or a 5-6 membered Heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂—C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″.

8. A compound in accordance with claim 7 wherein each R¹ is H or is independently selected from the group consisting of:
a) halo, OH, CN, NH₂, and
b) phenyl or a 5-6 membered Heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CN; NH₂;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN; and
iii) NR′C(O)R″, NR′SO₂R″, NR′CO₂R″ and NR′C(O)NR″R′″.

9. A compound in accordance with claim 1 wherein X represents a carbon atom.

10. A compound in accordance with claim 1 wherein X represents a nitrogen atom.

11. A compound in accordance with claim 1 wherein R² and R³ are independently H, C₁₋₃alkyl or haloC₁₋₃alkyl.

12. A compound in accordance with claim 11 wherein R² and R³ are independently H or methyl.

13. A compound in accordance with claim 1 wherein n is 2.

14. A compound in accordance with claim 1 wherein Z is Aryl optionally substituted with 1-3 halo groups and 0-1 groups selected from C₁₋₃alkyl and haloC₁₋₃alkyl.

15. A compound in accordance with claim 1 wherein Z is Heteroaryl optionally substituted with 1-3 halo groups and 0-1 groups selected from C₁₋₃alkyl and haloC₁₋₃alkyl.

16. A compound in accordance with claim 1 wherein R⁴ is H, fluoro or methyl optionally substituted with 1-3 halo groups.

17. A compound in accordance with claim 1 wherein R⁵ represents —CO₂H.

18. A compound in accordance with claim 1 wherein:
ring A is selected from the group consisting of: phenyl, naphthyl, oxadiazolyl, pyrazolyl and thiazolyl;
each R¹ is H or is independently selected from the group consisting of:
a) halo, OH, CN, NH₂, and
b) phenyl or a 5-6 membered Heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CN; NH₂;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN; and
iii) NR′C(O)R″, NR′SO₂R″, NR′CO₂R″ and NR′C(O)NR″R′″ wherein
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C-haloalkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₄alkoxy groups;
(b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;
and R′″ representing H or R″;
R² and R³ are independently H or methyl;
n is 2;
Z is Aryl or Heteroaryl optionally substituted with 1-3 halo groups and 0-1 groups selected from C₁₋₃alkyl and haloC₁₋₃alkyl;
R⁴ is H, fluoro or methyl optionally substituted with 1-3 halo groups, and
R⁵ represents —CO₂H.

19. A compound in accordance with claim 1 selected from the following table:
TABLE 1 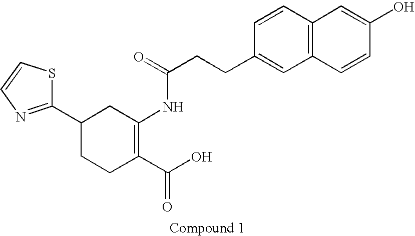 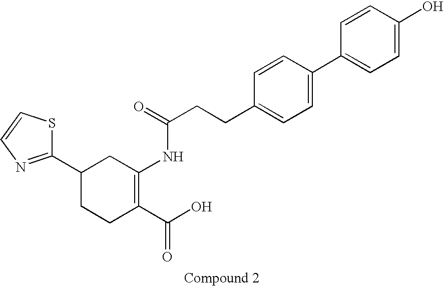 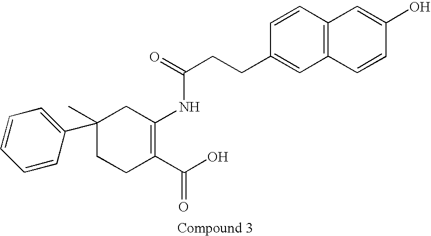 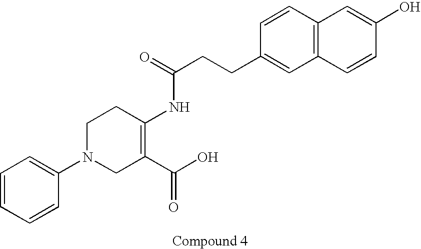 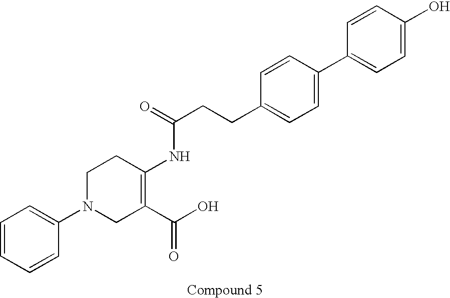 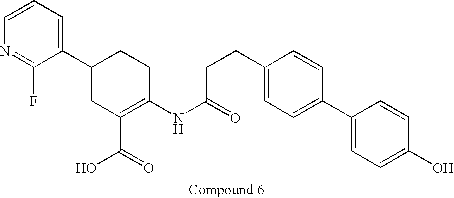 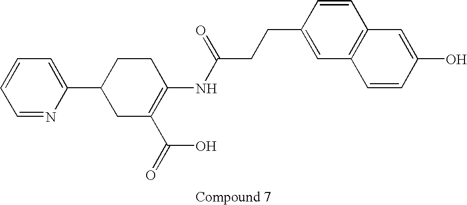 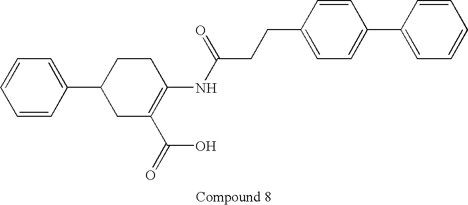 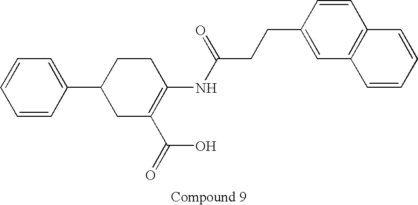 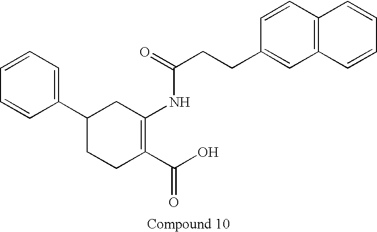 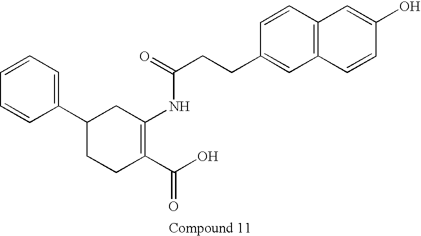 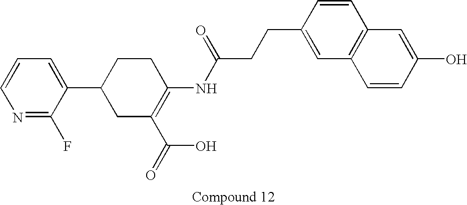 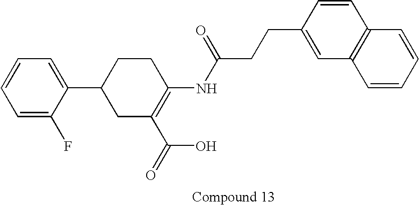 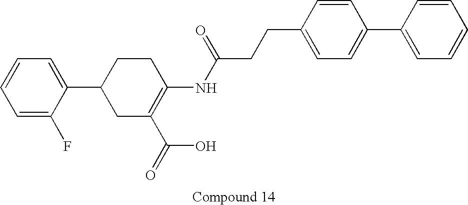 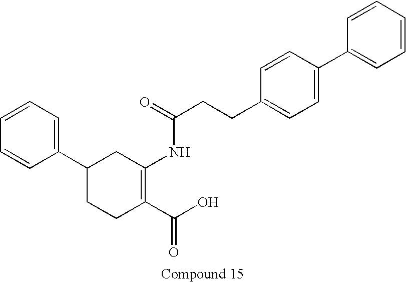 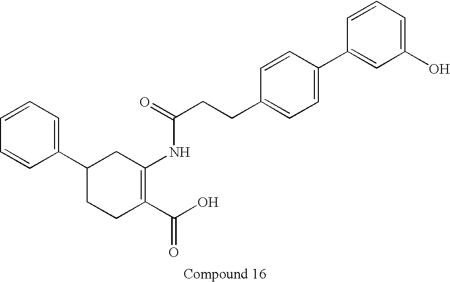 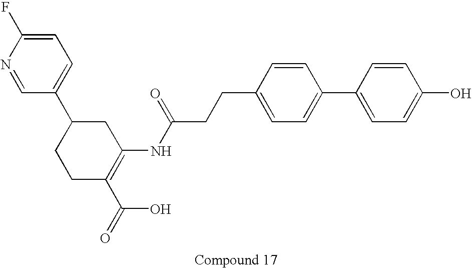 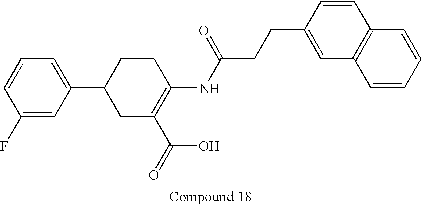 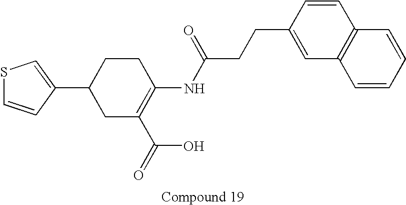 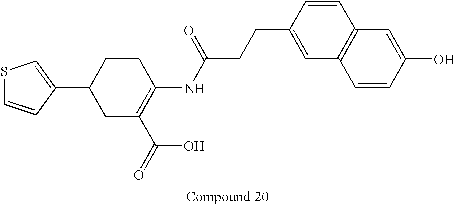 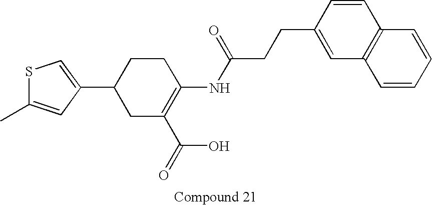 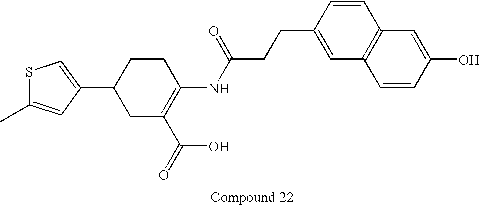 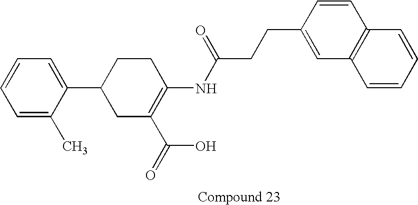 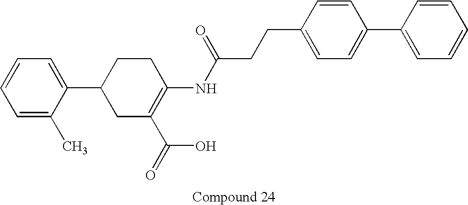 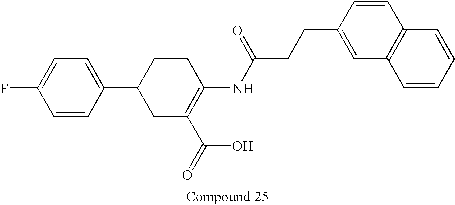 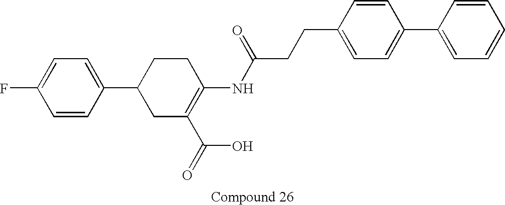 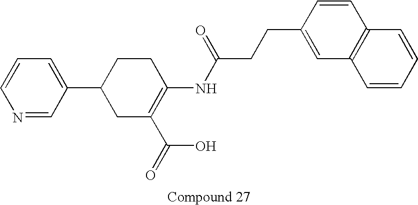 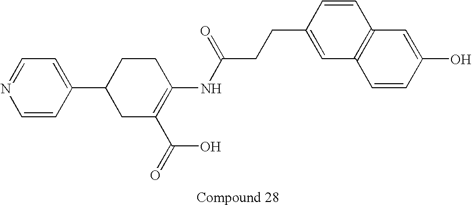 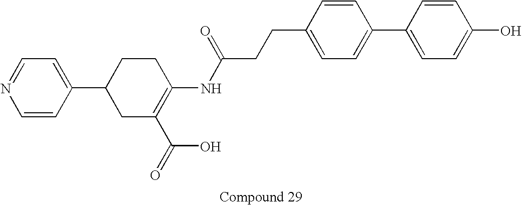 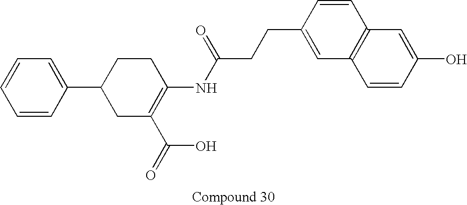 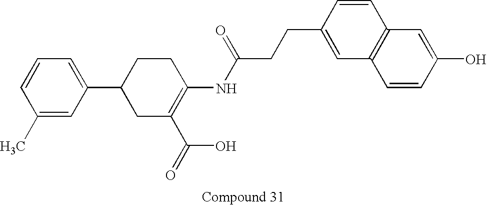 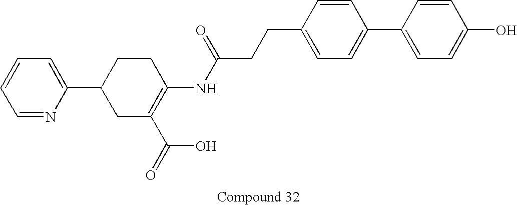 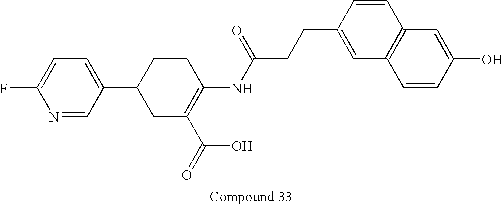 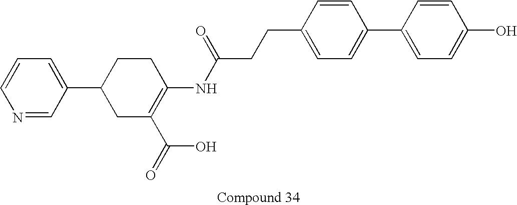 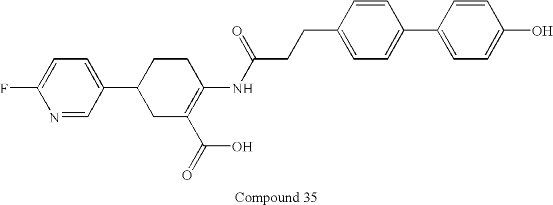 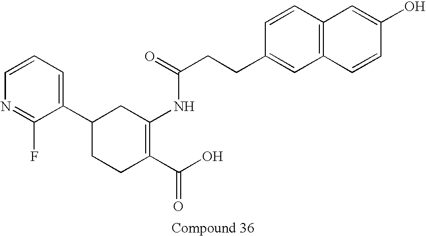 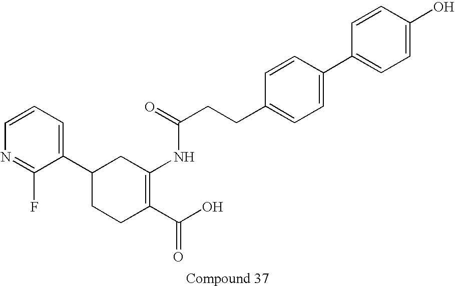 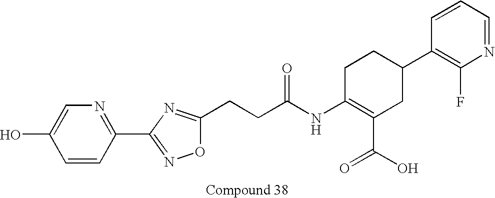 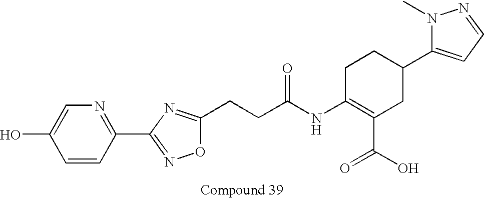 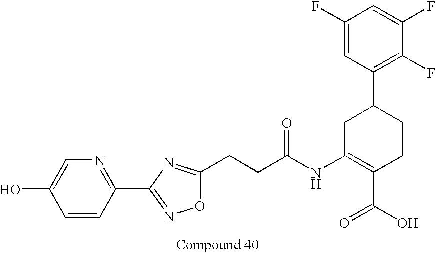 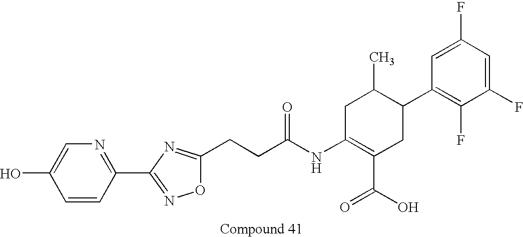 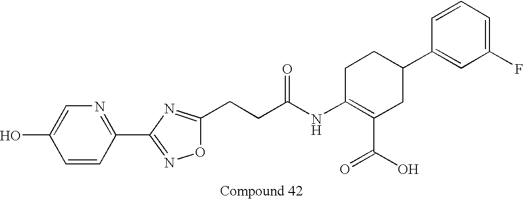 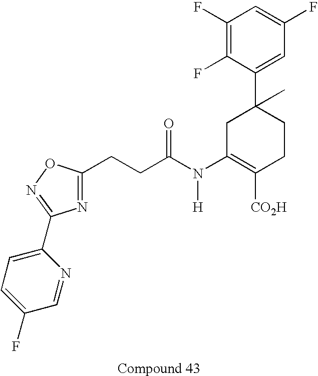 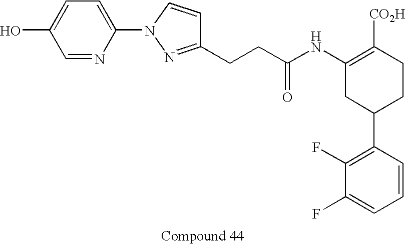 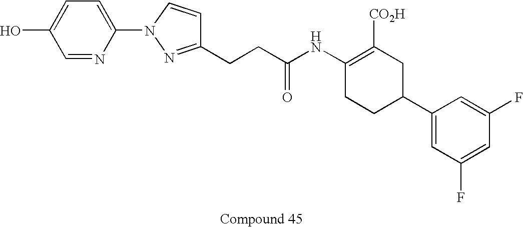
or a pharmaceutically acceptable salt or solvate thereof.

20. A pharmaceutical composition comprising a compound in accordance with claim 1 in combination with a pharmaceutically acceptable carrier.

21. A method of treating atherosclerosis in a human patient in need of such treatment comprising administering to the patient a compound of claim 1 in an amount that is effective for treating atherosclerosis.

22. A method of treating dyslipidemia in a human patient in need of such treatment comprising administering to the patient a compound of claim 1 in an amount that is effective for treating dyslipidemias.

23. A method of treating diabetes in a human patient in need of such treatment comprising administering to the patient a compound of claim 1 in an amount that is effective for treating diabetes.

24. A method of treating metabolic syndrome in a human patient in need of such treatment comprising administering to the patient a compound of claim 1 in an amount that is effective for treating metabolic syndrome.

25. A method of treating atherosclerosis, dyslipidemias, diabetes, metabolic syndrome or a related condition in a human patient in need of such treatment, comprising administering to the patient a compound of claim 1 and a DP receptor antagonist, said compounds being administered in an amount that is effective to treat atherosclerosis, dyslipidemia, diabetes or a related condition in the absence of substantial flushing.

26. A method of treatment in accordance with claim 25 wherein the DP receptor antagonist selected from the group consisting of compounds A through AJ: 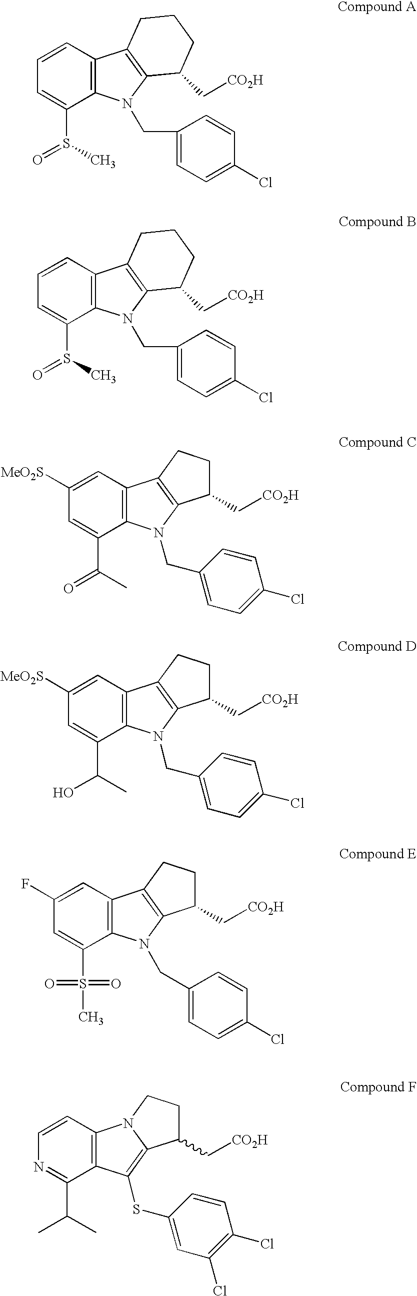 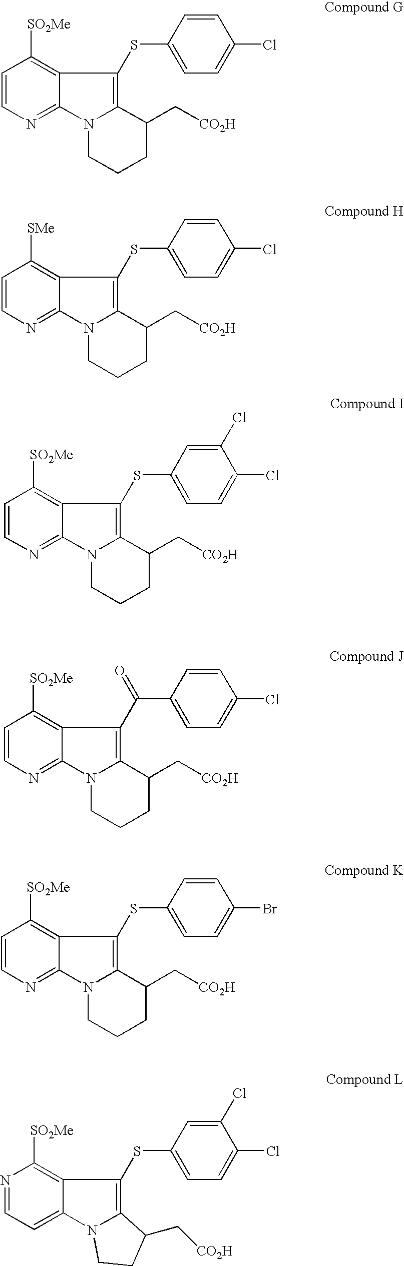 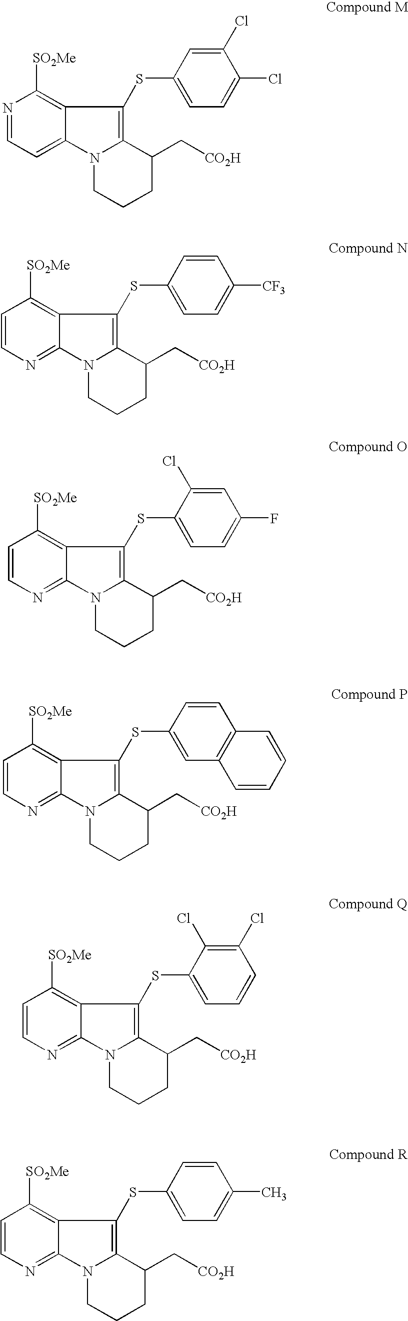 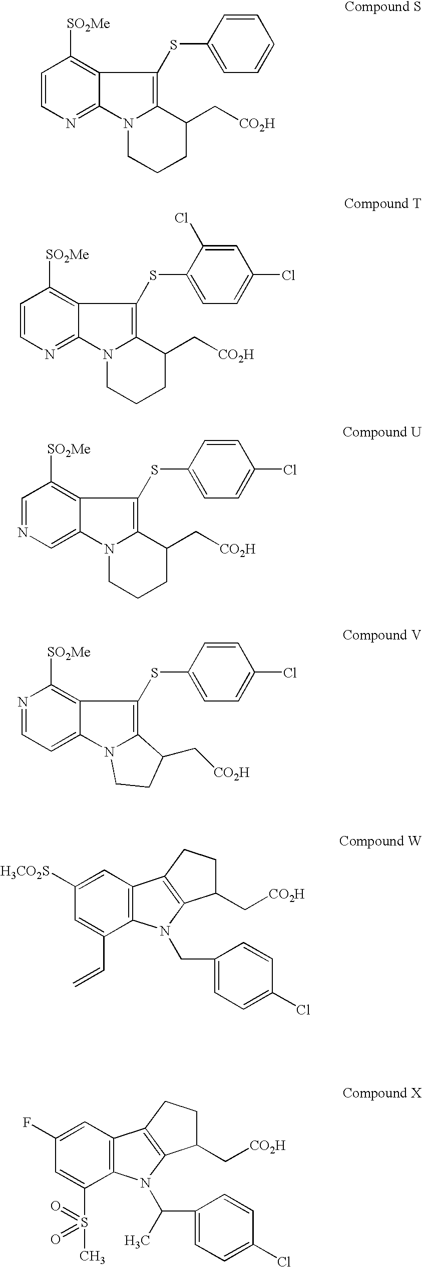 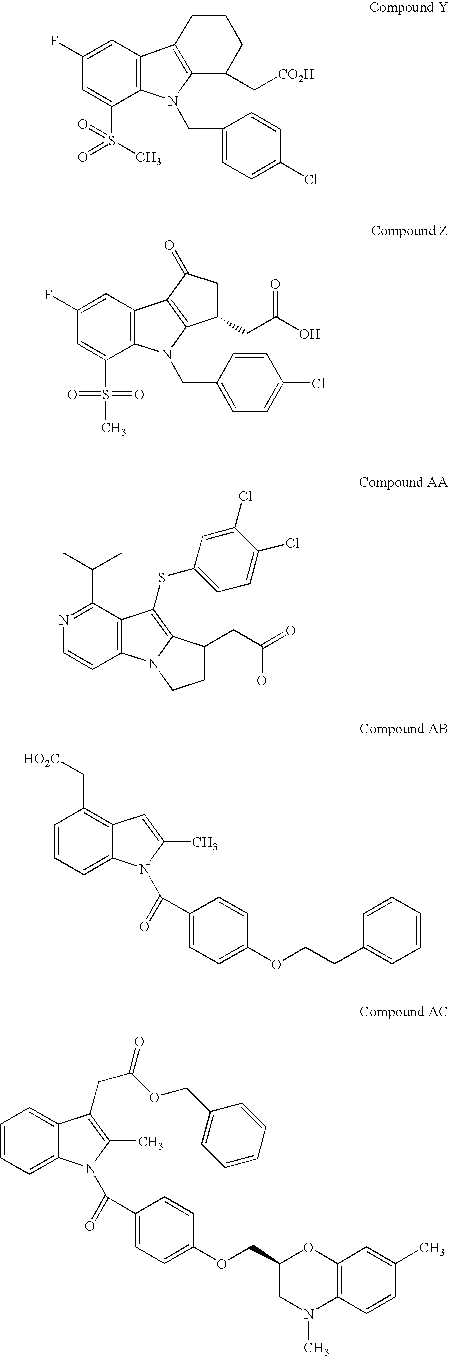 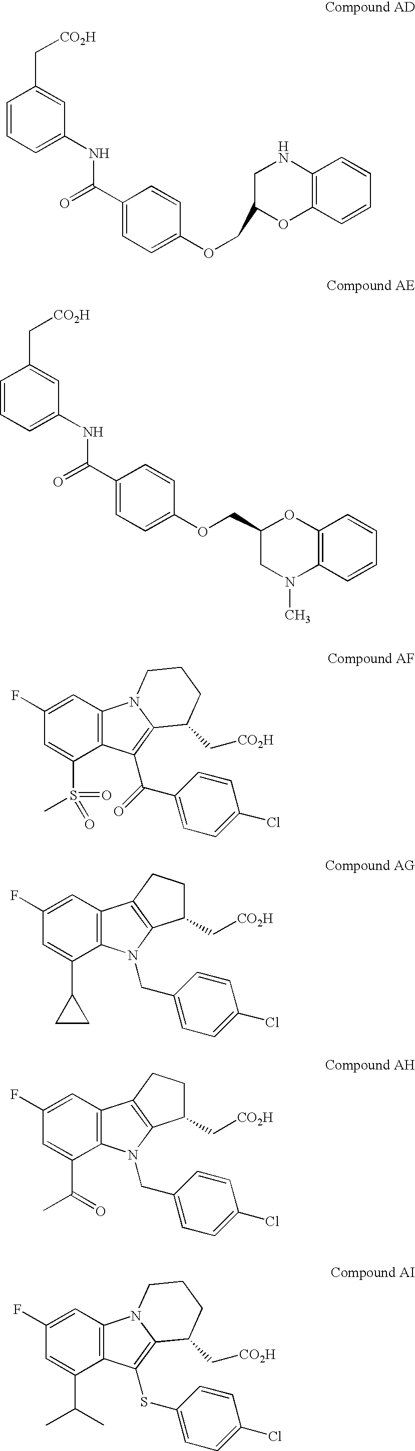 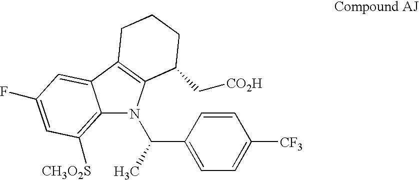
or a pharmaceutically acceptable salt or solvate thereof
